SEQUENCE LISTING (3 sequences)

SEQ ID NO: 1 - DNA, 20 bp, synthetic construct
  source 1..20: mol_type = other DNA; note = a sequence for a forward primer using Preparation Example 16
gcgggagatgatatggactt

SEQ ID NO: 2 - DNA, 21 bp, synthetic construct
  source 1..21: mol_type = other DNA; note = a sequence for a reverse primer using Preparation Example 16
ccgtctgagatgcatgaatac

SEQ ID NO: 3 - DNA, 23 bp, synthetic construct
  source 1..23: mol_type = other DNA; note = a sequence for a probe using Preparation Example 16
ccatctttatcggtacgccgccc

(19) United States
(12) Patent Application Publication
(10) Pub. No.: US 2024/0401112 A1
(43) Pub. Date: Dec. 5, 2024

(54) NOVEL COMPOUNDS AND THEIR APPLICATIONS

(71) Applicant: SFC CO., LTD., Cheongju-si (KR)

(72) Inventors: Do Min LEE, Cheongju-si (KR); Ho Young SI, Cheongju-si (KR); Goutam MASANTA, Cheongju-si (KR); Min Su EUM, Cheongju-si (KR); Ju Man SONG, Cheongju-si (KR); Bong Ki SHIN, Cheongju-si (KR); Jong Tae JE, Cheongju-si (KR)

(73) Assignee: SFC CO., LTD., Cheongju-si (KR)

(21) Appl. No.: 18/780,234

(22) Filed: Jul. 22, 2024

Related U.S. Application Data
(63) Continuation of application No. PCT/KR2023/001643, filed on Feb. 6, 2023.

(30) Foreign Application Priority Data
Feb. 10, 2022 (KR) 10-2022-0017565

(51) Int. Cl. C12Q 1/6818; C09B 11/04; C12Q 1/686; C12Q 1/6876

(52) U.S. Cl. CPC C12Q 1/6818; C09B 11/04; C12Q 1/686; C12Q 1/6876

(57) ABSTRACT

The present invention relates to a novel compound and uses thereof, and more specifically, to a compound capable of labeling biomolecules (such as nucleic acids and proteins), a composition for labeling or detecting biomolecules containing the compound, a support for labeling or detecting biomolecules containing the compound, and a method of labeling or detecting biomolecules using the compound.

### CROSS-REFERENCE TO RELATED APPLICATION

This application is based on the PCT Application No. PCT/KR2023/001643, filed on Feb. 6, 2023, and claims the benefit of priority from the prior Korean Patent Application No. 10-2022-0017565, filed on Feb. 10, 2022, the disclosures of which are incorporated herein by reference in its entirety.

### REFERENCE TO AN ELECTRONIC SEQUENCE LISTING

The entire contents of the electronic file identified by its name 602800-000106_Sequence List.xml, creation date Jul. 23, 2024, and size 4,323 bytes are incorporated herein by reference. Neither the presence nor absence of information that is not required under the sequence rules will create a presumption that such information is necessary to satisfy any of the requirements of 35 U.S.C. 112. Further, the grant of a patent on an application that is subject to the sequence rules (37 CFR 1.831 et seq.) constitutes a presumption that the granted patent complies with the requirements of these rules.

### BACKGROUND

### 1. Field of the Invention

The present invention relates to a novel compound and uses thereof, and more specifically, to a compound capable of labeling biomolecules (such as nucleic acids and proteins), a composition for labeling or detecting biomolecules containing the compound, a support for labeling or detecting biomolecules containing the compound, and a method of labeling or detecting biomolecules using the compound.

### 2. Discussion of Related Art

In the field of biotechnology, fluorescent dyes are used as a means for visualization to observe biological phenomena at the cellular level in vivo and in vitro, or to perform bio-imaging or examine a diseased area.

While there are self-emitting biomolecules such as a green fluorescent protein (GFP), generally, tissue or cells in the body, and lower-level biomolecules are stained with fluorescent dyes, or biomolecules such as proteins or nucleic acids are labeled with fluorescent dyes, and then imaging data is obtained by various techniques using optical equipment that is able to detect a fluorescent signal.

As mainly used optical analysis instruments, in addition to instruments for research purposes, such as a fluorescence microscope and a confocal microscope for cell observation, a flow cytometry, a microarray, a quantitative PCR system, an electrophoresis device for nucleic acid and protein separation and analysis, and a real-time in vivo imaging system, equipment for diagnosis and treatment including an in vitro diagnosis instrument based on a nucleic acid and protein diagnostic kit (or biochip), incorporating an immunoassay or PCR analysis and statistical technology, and operating tables and endoscopic equipment for medical image-guided surgery are known, and new applications and equipment with higher levels of resolution and data processing capability are continuously being developed.

To apply a fluorescent dye in the bio field, a fluorescent dye generally has less photo bleaching and quenching phenomena when present in a medium containing most biomolecules, that is, an aqueous solution, has a high molecular extinction coefficient and high quantum efficiency, and is stable under various pH conditions.

While various fluorescent dyes have been used in various research fields, those that satisfy all of the above-mentioned conditions are rare in the bio field, and currently used representative dyes include coumarin, cyanine, BODIPY, fluorescein, rhodamine, pyrene, carbopyronine, oxazine, xanthene, thioxanthene, and acridine. Among these, rhodamine derivatives and polymethine-based cyanine derivatives are particularly used.

Particularly, a cyanine-based fluorescent dye has polymethine which is connected at both ends with a nitrogen-containing heterocyclic ring and not only has the structural advantage of being able to emit various types of fluorescence across the entire visible light range of 450 to 800 nm and the near-infrared range by controlling a polymethine length but also generally has the optical property of having a very high extinction coefficient.

### SUMMARY OF THE INVENTION

The present invention is directed to providing a novel compound that can be widely used to observe and identify biomolecules in the optical imaging field.

The present invention is also directed to providing a composition for labeling or detecting biomolecules containing the compound, a support for labeling or detecting biomolecules containing the compound, and a method of labeling or detecting biomolecules using the compound, as various uses of the novel compound defined herein.

According to an aspect of the prevent invention to solve the above technical problems, there is provided a compound represented by Chemical Formula 1 below.

### [Chemical Formula 1]

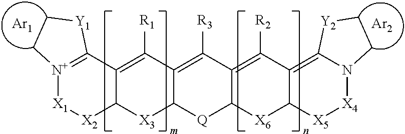

Wherein,
Ar₁ and Ar₂ are each independently a substituted or unsubstituted C₆-C₂₀ aryl or a substituted or unsubstituted C₁-C₂₀ heteroaryl containing at least one heteroatom,
Y₁ and Y₂ are each independently CRₐR_{b}, NR_{c}, O, or S,
Q is O, S, or NR_{d},
X₁ to X₆ are each independently CRₑR_{f},
Rₐ to R_{f} and R₁ to R₃ are each independently
(1) a functional group selected from hydrogen, deuterium, a substituted or unsubstituted C₁-C₄₀ alkyl, a substituted or unsubstituted C₁-C₄₀ heteroalkyl containing at least one heteroatom, a substituted or unsubstituted C₂-C₄₀ alkenyl, a substituted or unsubstituted C₂-C₄₀ alkynyl, a substituted or unsubstituted C₃-C₂₀ cycloalkyl, a substituted or unsubstituted C₃-C₂₀ cycloalkenyl, a substituted or unsubstituted C₂-C₂₀ heterocycloalkyl, hydroxy, oxido (—O⁻), a substituted or unsubstituted C₁-C₄₀ alkoxy, a substituted or unsubstituted C₃-C₄₀ cycloalkyloxy, a substituted or unsubstituted C₅-C₄₀ aryloxy, a substituted or unsubstituted C₂-C₄₀ heteroaryloxy, a substituted or unsubstituted C₅-C₅₀ aryl, a substituted or unsubstituted C₂-C₅₀ heteroaryl, a substituted or unsubstituted C₅-C₅₀ aralkyl, a substituted or unsubstituted C₁-C₄₀ alkylthio, a substituted or unsubstituted C₅-C₄₀ arylthio, a substituted or unsubstituted C₃-C₄₀ cycloalkylthio, a substituted or unsubstituted C₂-C₄₀ heteroarylthio, a substituted or unsubstituted acylamino, acyloxy, a substituted or unsubstituted phosphino, carboxylate (—CO₂⁻), trifluoromethylsulfonyl (—SO₂CF₃), a substituted or unsubstituted ammonium, nitro, sulfonic acid (—SO₃H), sulfonate, a substituted sulfonyl, a substituted sulfonic acid ester, a substituted or unsubstituted sulfonamide, a substituted thioketone, a trihalomethyl (—CF₃, —CCl₃, —CBr₃, —CI₃), a haloformyl (—COCl, —COBr, —COI), formyl (—CHO), acyl, carboxyl, a substituted ester, a substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, a substituted or unsubstituted silyl, a substituted or unsubstituted amide, carbamate, carboxylate, a substituted or unsubstituted phosphine, substituted or unsubstituted phosphoric acid, phosphate, phosphonic acid, phosphonate, nitrile, hydrazine, acetal, ketal, and polyalkylene oxide,
(2) a functional group selected from carboxyl, a carboxyl derivative, hydroxyl, a haloalkyl, a dienophile, an aldehyde, a substituted ketone, a sulfonyl halide, a thiol, amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the functional group,
(3) a reactive group capable of covalent bonding with a functional group selected from carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the reactive group, or
(4) a protecting group selected from an alcohol-derived protecting group, an amine-derived protecting group, a carbonyl-derived protecting group, a carboxylic acid-derived protecting group, a phosphate-derived protecting group, and an alkyne-derived protecting group, or any functional group substituted with the protecting group,
m and n are each independently 0 or 1, and
Rₑ and R_{f} of at least one of X₁ to X₆ are not hydrogen and deuterium.

Specifically, the compound may be represented by at least one of Chemical Formulas 2 to 4 below.

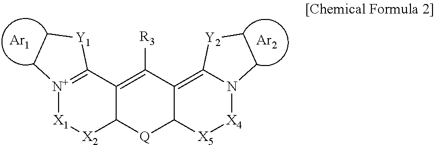

Wherein,
Rₑ and R_{f} of at least one of X₁, X₂, X₄, and X₅ are not hydrogen and deuterium.

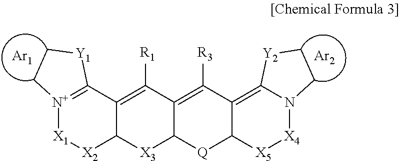

Wherein,
Rₑ and R_{f} of at least one of X₁ to X₅ are not hydrogen and deuterium.

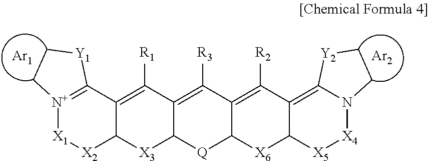

Wherein,
Rₑ and R_{f} of at least one of X₁ to X₆ are not hydrogen and deuterium.

The compound may be represented by Chemical Formula 5 below.

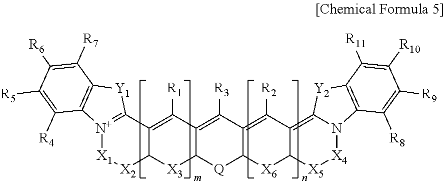

Wherein,
Y₁ and Y₂ are each independently CRₐR_{b}, NR_{c}, O, or S,
Q is O, S, or NR_{d},
X₁ to X₆ are each independently CRₑR_{f},
Rₐ to R_{f} and R₁ to R₁₁ are each independently
(1) a functional group selected from hydrogen, deuterium, a substituted or unsubstituted C₁-C₄₀ alkyl, a substituted or unsubstituted C₁-C₄₀ heteroalkyl containing at least one heteroatom, a substituted or unsubstituted C₂-C₄₀ alkenyl, a substituted or unsubstituted C₂-C₄₀ alkynyl, a substituted or unsubstituted C₃-C₂₀ cycloalkyl, a substituted or unsubstituted C₃-C₂₀ cycloalkenyl, a substituted or unsubstituted C₂-C₂₀ heterocycloalkyl, hydroxy, oxido (—O⁻), a substituted or unsubstituted C₁-C₄₀ alkoxy, a substituted or unsubstituted C₃-C₄₀ cycloalkyloxy, a substituted or unsubstituted C₅-C₄₀ aryloxy, a substituted or unsubstituted C₂-C₄₀ heteroaryloxy, a substituted or unsubstituted C₅-C₅₀ aryl, a substituted or unsubstituted C₂-C₅₀ heteroaryl, a substituted or unsubstituted C₅-C₅₀ aralkyl, a substituted or unsubstituted C₁-C₄₀ alkylthio, a substituted or unsubstituted C₅-C₄₀ arylthio, a substituted or unsubstituted C₃-C₄₀ cycloalkylthio, a substituted or unsubstituted C₂-C₄₀ heteroarylthio, a substituted or unsubstituted acylamino, acyloxy, a substituted or unsubstituted phosphino, carboxylate (—CO₂⁻), trifluoromethylsulfonyl (—SO₂CF₃), a substituted or unsubstituted ammonium, nitro, sulfonic acid (—SO₃H), sulfonate, a substituted sulfonyl, a substituted sulfonic acid ester, a substituted or unsubstituted sulfonamide, a substituted thioketone, a trihalomethyl (—CF₃, —CCl₃, —CBr₃, —CI₃), a haloformyl (—COCl, —COBr, —COI), formyl (—CHO), acyl, carboxyl, a substituted ester, a substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, a substituted or unsubstituted silyl, a substituted or unsubstituted amide, carbamate, carboxylate, a substituted or unsubstituted phosphine, substituted or unsubstituted phosphoric acid, phosphate, phosphonic acid, phosphonate, nitrile, hydrazine, acetal, ketal, and polyalkylene oxide,
(2) a functional group selected from carboxyl, a carboxyl derivative, hydroxyl, a haloalkyl, a dienophile, an aldehyde, a substituted ketone, a sulfonyl halide, a thiol, amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the functional group,
(3) a reactive group capable of covalent bonding with a functional group selected from carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the reactive group, or
(4) a protecting group selected from an alcohol-derived protecting group, an amine-derived protecting group, a carbonyl-derived protecting group, a carboxylic acid-derived protecting group, a phosphate-derived protecting group, and an alkyne-derived protecting group, or any functional group substituted with the protecting group,
m and n are each independently 0 or 1, and
Rₑ and R_{f} of at least one of X₁ to X₆ are not hydrogen and deuterium.

The compounds of the above-described Chemical Formulas 1 to 5 may be present in the form of a resonance structure depending on the movement of electrons.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIGS. 1 to 3 are graphs showing the results of real-time PCR repeated twice using the dual-labeled probes of Examples 1 to 9 and Comparative Examples 1 to 3 for a black queen cell virus (BQCV) target; and

FIGS. 4 to 6 show the average RFU values of real-time PCR repeated twice using the dual-labeled probes of Examples 1 to 9 and Comparative Examples 1 to 3 for a BQCV target.

### DETAILED DESCRIPTION OF EXEMPLARY EMBODIMENTS

In order to better understand the present invention, certain terms are defined herein for convenience. Unless defined otherwise herein, scientific and technical terms used herein will have meanings commonly understood by those skilled in the art.

In addition, unless specifically indicated otherwise, terms in a singular form also include plural forms, and terms in a plural form should be understood to include singular forms as well.

### Novel Compound

According to one aspect of the present invention, a novel compound represented by the following Chemical Formula 1 is provided.

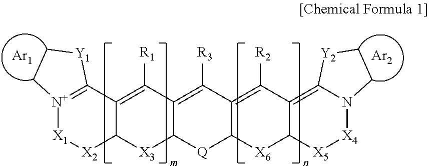

Wherein,
Ar₁ and Ar₂ are each independently a substituted or unsubstituted C₆-C₂₀ aryl or a substituted or unsubstituted C₁-C₂₀ heteroaryl containing at least one heteroatom,
Y₁ and Y₂ are each independently CRₐR_{b}, NR_{c}, O, or S,
Q is O, S, or NR_{d},
X₁ to X₆ are each independently CRₑR_{f},
Rₐ to R_{f} and R₁ to R₃ are each independently
(1) a functional group selected from hydrogen, deuterium, a substituted or unsubstituted C₁-C₄₀ alkyl, a substituted or unsubstituted C₁-C₄₀ heteroalkyl containing at least one heteroatom, a substituted or unsubstituted C₂-C₄₀ alkenyl, a substituted or unsubstituted C₂-C₄₀ alkynyl, a substituted or unsubstituted C₃-C₂₀ cycloalkyl, a substituted or unsubstituted C₃-C₂₀ cycloalkenyl, a substituted or unsubstituted C₂-C₂₀ heterocycloalkyl, hydroxy, oxido (—O⁻), a substituted or unsubstituted C₁-C₄₀ alkoxy, a substituted or unsubstituted C₃-C₄₀ cycloalkyloxy, a substituted or unsubstituted C₅-C₄₀ aryloxy, a substituted or unsubstituted C₂-C₄₀ heteroaryloxy, a substituted or unsubstituted C₅-C₅₀ aryl, a substituted or unsubstituted C₂-C₅₀ heteroaryl, a substituted or unsubstituted C₅-C₅₀ aralkyl, a substituted or unsubstituted C₁-C₄₀ alkylthio, a substituted or unsubstituted C₅-C₄₀ arylthio, a substituted or unsubstituted C₃-C₄₀ cycloalkylthio, a substituted or unsubstituted C₂-C₄₀ heteroarylthio, a substituted or unsubstituted acylamino, acyloxy, a substituted or unsubstituted phosphino, carboxylate (—CO₂⁻), trifluoromethylsulfonyl (—SO₂CF₃), a substituted or unsubstituted ammonium, nitro, sulfonic acid (—SO₃H), sulfonate, a substituted sulfonyl, a substituted sulfonic acid ester, a substituted or unsubstituted sulfonamide, a substituted thioketone, a trihalomethyl (—CF₃, —CCl₃, —CBr₃, —CI₃), a haloformyl (—COCl, —COBr, —COI), formyl (—CHO), acyl, carboxyl, a substituted ester, a substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, a substituted or unsubstituted silyl, a substituted or unsubstituted amide, carbamate, carboxylate, a substituted or unsubstituted phosphine, substituted or unsubstituted phosphoric acid, phosphate, phosphonic acid, phosphonate, nitrile, hydrazine, acetal, ketal, and polyalkylene oxide,
(2) a functional group selected from carboxyl, a carboxyl derivative, hydroxyl, a haloalkyl, a dienophile, an aldehyde, a substituted ketone, a sulfonyl halide, a thiol, amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the functional group,
(3) a reactive group capable of covalent bonding with a functional group selected from carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the reactive group, or
(4) a protecting group selected from an alcohol-derived protecting group, an amine-derived protecting group, a carbonyl-derived protecting group, a carboxylic acid-derived protecting group, a phosphate-derived protecting group, and an alkyne-derived protecting group, or any functional group substituted with the protecting group.

When Ar₁ and/or Ar₂ are a substituted aryl, the functional group bonded to any carbon of Ar₁ and/or Ar₂ may be a functional group listed in (1) to (4) above or any functional group substituted with a functional group listed in (1) to (4) above.

In Chemical Formula 1, m and n refer to the number of rings present between the ring containing Q and the ring containing N, and may each be independently 0 or 1.

In Chemical Formula 1, Rₑ and R_{f} of at least one of X₁ to X₆ are not hydrogen and deuterium. In other words, in Chemical Formula 1, X₁ to X₆ are carbons to which any functional group is bonded, and at least one of X₁ to X₆ may be present as a carbon doubly substituted with a functional group other than hydrogen. Since Rₑ and R_{f} of at least one of X₁ to X₆ in Chemical Formula 1 are present in a state other than hydrogen and deuterium, it is possible to increase the solubility of the compound in various solvents. As the solubility of the compound is increased, the handleability of the compound may be much easier.

In Chemical Formula 1, any functional group substituted with a functional group selected from (2) carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite may be selected from the functional groups listed in (1), (3), and (4) above.

In Chemical Formula 1, any functional group substituted with a reactive group capable of covalent bonding with a functional group selected from (3) carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite may be selected from the functional groups listed in (1), (2), and (4) above.

In Chemical Formula 1, any functional group substituted with a protecting group selected from (4) an alcohol-derived protecting group, an amine-derived protecting group, a carbonyl-derived protecting group, a carboxylic acid-derived protecting group, a phosphate-derived protecting group, and an alkyne-derived protecting group may be selected from the functional groups listed in (1) to (3) above.

The compound represented by Chemical Formula 1 may be present in the form of a resonance structure as shown in Chemical Formula 1-1 below depending on the movement of electrons within the compound.

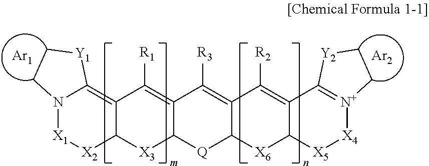

The compound represented by Chemical Formula 1 may be present as an isomer of Chemical Formula 1.

In an embodiment, when m and n in Chemical Formula 1 are both 0, the compound may be represented by Chemical Formula 2 or Chemical Formula 2-1 (the resonance structure of Chemical Formula 2) below.

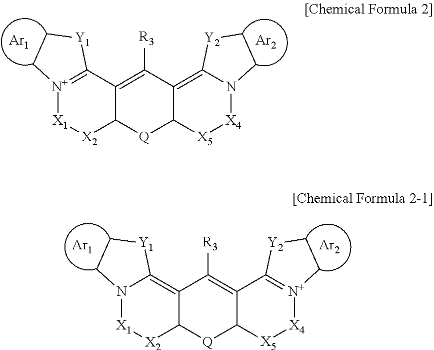

According to the above-described definition for the compound, Rₑ and R_{f} of at least one of X₁, X₂, X₄, and X₅ are not hydrogen and deuterium.

In another embodiment, when m and n in Chemical Formula 1 are 1 and 0, respectively, the compound may be represented by Chemical Formula 3 or Chemical Formula 3-1 (the resonance structure of Chemical Formula 3) below. When m and n in Chemical Formula 1 are 0 and 1, respectively, the compound may be present as an isomer of Chemical Formula 3 or Chemical Formula 3-1 (the resonance structure of Chemical Formula 3) below.

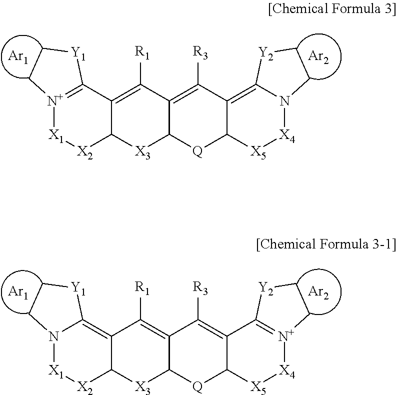

According to the above-described definition of the compound, Rₑ and R_{f} of at least one of X₁ to X₅ are not hydrogen and deuterium.

In another embodiment, when m and n in Chemical Formula 1 are both 1, the compound may be represented by Chemical Formula 4 or Chemical Formula 4-1 (the resonance structure of Chemical Formula 4) below.

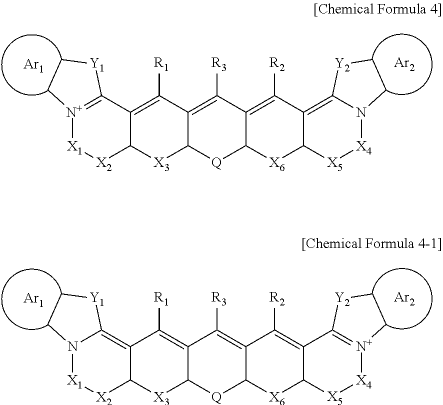

According to the above-described definition of the compound, Rₑ and R_{f} of at least one of X₁ to X₆ are not hydrogen and deuterium.

In an embodiment, when any functional group among Rₐ to R_{f} and R₁ to R₃ is a substituted functional group, any carbon of at least one of the functional groups may be a functional group listed in (1) to (4) above or any functional group substituted with a functional group listed in (1) to (4).

In another embodiment, at least one of the functional group bonded to any carbon of Rₐ to R_{f}, R₁ to R₃, and Ar₁ and the functional group bonded to any carbon of Ar₂ may be (2) a functional group selected from carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the functional group, (3) a reactive group capable of covalent bonding with a functional group selected from carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the reactive group, or (4) a protecting group selected from an alcohol-derived protecting group, an amine-derived protecting group, a carbonyl-derived protecting group, a carboxylic acid-derived protecting group, a phosphate-derived protecting group, and an alkyne-derived protecting group, or any functional group substituted with the protecting group.

At least one selected from Rₑ and R_{f} of at least one of X₁ to X₆ may be a substituted or unsubstituted C₁-C₄₀ alkyl, a substituted or unsubstituted C₁-C₄₀ heteroalkyl containing at least one heteroatom, a substituted or unsubstituted C₃-C₂₀ cycloalkyl, a substituted or unsubstituted C₃-C₃₀ heterocycloalkyl containing at least one heteroatom, a substituted or unsubstituted C₅-C₅₀ aryl, a substituted or unsubstituted C₂-C₅₀ heteroaryl, fluoro (—F), chloro (—Cl), bromo (—Br), or iodo (—I).

More specifically, the compound may be represented by Chemical Formula 5 below. With respect to the compound represented by Chemical Formula 5 below, unless otherwise defined, the content overlapping with the above-described Chemical Formula 1 may be interpreted in the same way.

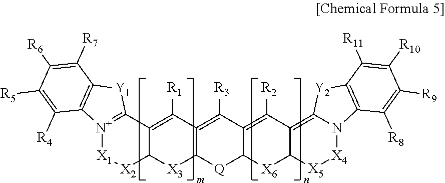

Wherein,
Y₁ and Y₂ are each independently CRₐR_{b}, NR_{c}, O, or S,
Q is O, S, or NR_{d},
X₁ to X₆ are each independently CRₑR_{f},
Rₐ to R_{f} and R₁ to Rn₁₁ are each independently
(1) a functional group selected from hydrogen, deuterium, a substituted or unsubstituted C₁-C₄₀ alkyl, a substituted or unsubstituted C₁-C₄₀ heteroalkyl containing at least one heteroatom, a substituted or unsubstituted C₂-C₄₀ alkenyl, a substituted or unsubstituted C₂-C₄₀ alkynyl, a substituted or unsubstituted C₃-C₂₀ cycloalkyl, a substituted or unsubstituted C₃-C₂₀ cycloalkenyl, a substituted or unsubstituted C₂-C₂₀ heterocycloalkyl, hydroxy, oxido (—O⁻), a substituted or unsubstituted C₁-C₄₀ alkoxy, a substituted or unsubstituted C₃-C₄₀ cycloalkyloxy, a substituted or unsubstituted C₅-C₄₀ aryloxy, a substituted or unsubstituted C₂-C₄₀ heteroaryloxy, a substituted or unsubstituted C₅-C₅₀ aryl, a substituted or unsubstituted C₂-C₅₀ heteroaryl, a substituted or unsubstituted C₅-C₅₀ aralkyl, a substituted or unsubstituted C₁-C₄₀ alkylthio, a substituted or unsubstituted C₅-C₄₀ arylthio, a substituted or unsubstituted C₃-C₄₀ cycloalkylthio, a substituted or unsubstituted C₂-C₄₀ heteroarylthio, a substituted or unsubstituted acylamino, acyloxy, a substituted or unsubstituted phosphino, carboxylate (—CO₂⁻), trifluoromethylsulfonyl (—SO₂CF₃), a substituted or unsubstituted ammonium, nitro, sulfonic acid (—SO₃H), sulfonate, a substituted sulfonyl, a substituted sulfonic acid ester, a substituted or unsubstituted sulfonamide, a substituted thioketone, a trihalomethyl (—CF₃, —CCl₃, —CBr₃, —CI₃), a haloformyl (—COCl, —COBr, —COI), formyl (—CHO), acyl, carboxyl, a substituted ester, a substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, a substituted or unsubstituted silyl, a substituted or unsubstituted amide, carbamate, carboxylate, a substituted or unsubstituted phosphine, substituted or unsubstituted phosphoric acid, phosphate, phosphonic acid, phosphonate, nitrile, hydrazine, acetal, ketal, and polyalkylene oxide,
(2) a functional group selected from carboxyl, a carboxyl derivative, hydroxyl, a haloalkyl, a dienophile, an aldehyde, a substituted ketone, a sulfonyl halide, a thiol, amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the functional group,
(3) a reactive group capable of covalent bonding with a functional group selected from carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the reactive group, or
(4) a protecting group selected from an alcohol-derived protecting group, an amine-derived protecting group, a carbonyl-derived protecting group, a carboxylic acid-derived protecting group, a phosphate-derived protecting group, and an alkyne-derived protecting group, or any functional group substituted with the protecting group,
m and n are 0 or 1, and
Rₑ and R_{f} of at least one of X₁ to X₆ are not hydrogen and deuterium.

Like Chemical Formula 1, the compound represented by Chemical Formula 5 may be present in the form of a resonance structure as shown in Chemical Formula 5-1 below depending on the movement of electrons within the compound.

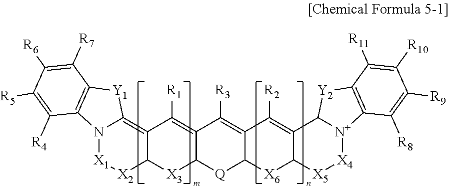

The compound represented by Chemical Formula 5 may be present as an isomer of Chemical Formula 5.

In an embodiment, when m and n in Chemical Formula 5 are both 0, the compound may be represented by Chemical Formula 6 or Chemical Formula 6-1 (the resonance structure of Chemical Formula 6) below.

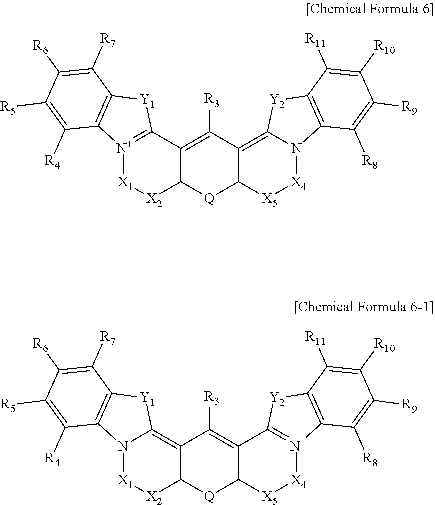

According to the above-described definition of the compound, Rₑ and R_{f} of at least one of X₁, X₂, X₄, and X₅ are not hydrogen and deuterium.

In another embodiment, when m and n in Chemical Formula 5 is 1 and 0, respectively, the compound may be represented by Chemical Formula 7 or Chemical Formula 7-1 (the resonance structure of Chemical Formula 7) below. When m and n in Chemical Formula 5 are 0 and 1, respectively, the compound may be present as an isomer of Chemical Formula 7 or Chemical Formula 7-1 (the resonance structure of Chemical Formula 7) below.

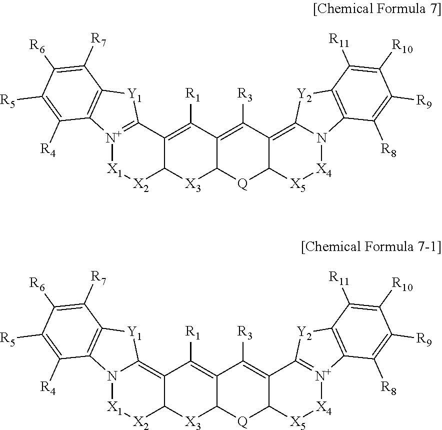

According to the above-described definition of the compound, Rₑ and R_{f} of at least one of X₁ to X₅ are not hydrogen and deuterium.

In another embodiment, when m and n in Chemical Formula 5 are both 1, the compound may be represented by Chemical Formula 8 or Chemical Formula 8-1 (the resonance structure of Chemical Formula 8) below.

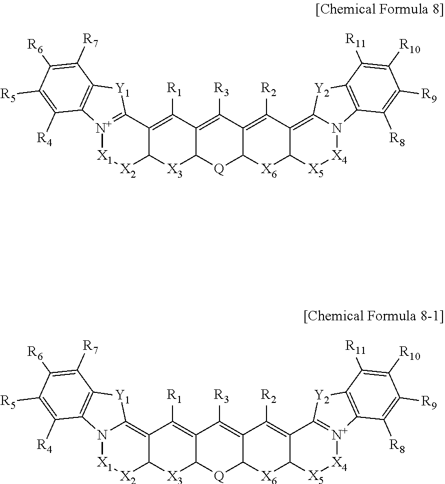

According to the above-described definition of the compound, Rₑ and R_{f} of at least one of X₁ to X₆ are not hydrogen and deuterium.

In an embodiment, at least one of Rₐ to R_{f} and R₁ to R₁₁ may be:

(2) a functional group selected from carboxyl, a carboxyl derivative, hydroxyl, a haloalkyl, a dienophile, an aldehyde, a substituted ketone, a sulfonyl halide, a thiol, amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the functional group,

(3) a reactive group capable of covalent bonding with a functional group selected from carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the reactive group; or (4) a protecting group selected from an alcohol-derived protecting group, an amine-derived protecting group, a carbonyl-derived protecting group, a carboxylic acid-derived protecting group, a phosphate-derived protecting group, and an alkyne-derived protecting group, or any functional group substituted with the protecting group.

R₄ to R₁₁ may be independently present as the functional groups defined above, but in some embodiments, two adjacent functional groups among R₄ to R₁₁ may be bonded and form a substituted or unsubstituted ring (such as a 4-membered ring, a 5-membered ring, a 6-membered ring, or a ring composed of 7 or more atoms, and a fused ring with multiple conjugated rings). In addition, the ring may be an aliphatic or aromatic ring and include at least one heteroatom.

When at least one of R₄ to R₁₁ is bonded to an adjacent substituent to form a substituted or unsubstituted ring, at least one of R₄ to R₁₁ may be bonded to an adjacent substituent through C, N, O, S, Se, or Si or directly bonded to the adjacent substituent by a single bond.

When at least one of R₄ to R₁₁ is bonded to an adjacent substituent to form a substituted ring, any carbon of at least one of the rings may be the functional group listed in (1) to (4) above or any functional group substituted with the functional group listed in (1) to (4).

Hereinafter, some functional groups mentioned herein will be described in detail. Functional groups not mentioned below follow the general definitions commonly used in the relevant technical field.

The phrase “substituted or unsubstituted” used herein means that any functional group may be present in an unsubstituted state or in a state of being substituted with at least one substituent within a range that does not reduce the effect of the compounds defined herein, and the phrase “substituted or unsubstituted” is a general expression commonly used in the relevant technical field in defining any functional group, and unless otherwise defined, the scope of any functional group interpreted as being “substituted or unsubstituted” also follows the general definitions commonly used in the relevant technical field.

When any functional group defined herein is a substituted functional group, any carbon of the functional group may be substituted with at least one substituent.

For example, the substituent may be selected from the functional groups listed in (1) to (4):

(1) a functional group selected from hydrogen, deuterium, a C₁-C₄₀ alkyl, a C₁-C₄₀ heteroalkyl containing at least one heteroatom, a C₂-C₄₀ alkenyl, a C₂-C₄₀ alkynyl, a C₃-C₂₀ cycloalkyl, a C₃-C₂₀ cycloalkenyl, a C₂-C₂₀ heterocycloalkyl, hydroxy, oxido (—O⁻), a C₁-C₄₀ alkoxy, a C₃-C₄₀ cycloalkyloxy, a C₅-C₄₀ aryloxy, a C₂-C₄₀ heteroaryloxy, a C₅-C₅₀ aryl, a C₂-C₅₀ heteroaryl, a C₅-C₅₀ aralkyl, a C₁-C₄₀ alkylthio, a C₅-C₄₀ arylthio, a C₃-C₄₀ cycloalkylthio, a C₂-C₄₀ heteroarylthio, acylamino, acyloxy, phosphino, carboxylate (—CO₂⁻), trifluoromethylsulfonyl (—SO₂CF₃), ammonium, nitro, sulfonic acid (—SO₃H), sulfonate, a substituted sulfonyl, a substituted sulfonic acid ester, sulfonamide, a substituted thioketone, a trihalomethyl (—CF₃, —CCl₃, —CBr₃, —CI₃), a haloformyl (—COCl, —COBr, —COI), formyl (—CHO), acyl, carboxyl, a substituted ester, aminocarbonyl, nitro, nitroso (—N═O), fluoro (—F), chloro (—Cl), bromo (—Br), iodo (—I), germanium, boron, aluminum, silyl, amide, carbamate, carboxylate, phosphine, phosphoric acid, phosphate, phosphonic acid, phosphonate, nitrile, hydrazine, acetal, ketal, and polyalkylene oxide;

(2) a functional group selected from carboxyl, a carboxyl derivative, hydroxyl, a haloalkyl, a dienophile, an aldehyde, a substituted ketone, a sulfonyl halide, a thiol, amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the functional group;

(3) a reactive group capable of covalent bonding with a functional group selected from carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the reactive group; or

(4) a protecting group selected from an alcohol-derived protecting group, an amine-derived protecting group, a carbonyl-derived protecting group, a carboxylic acid-derived protecting group, a phosphate-derived protecting group, and an alkyne-derived protecting group, or any functional group substituted with the protecting group.

The reactive group and the types of reactions caused by the reactive group herein are generally well known in the field of bioconjugate chemistry. The types of reactions include nucleophilic substitution (such as a reaction of an acyl halide and/or active ester with amine and/or alcohol), electrophilic substitution (such as an enamine reaction), and an addition reaction to carbon-heteroatom multiple bonds (such as the Michael reaction and the Diels-Alder addition).

As the reactive group, (a) a carboxyl group and its derivatives: N-hydroxysuccinimide esters, pentafluorophenyl esters, tetrafluorophenyl esters, sulfotetrafluorophenyl esters, N-hydroxybenztriazole esters, acyl halides, acyl imidazoles, thioesters, p-nitrophenyl esters, alkyl esters, alkenyl esters, alkynyl esters, and aromatic esters; (b) a hydroxyl which may be converted to esters, ethers, and aldehydes; (c) a haloalkyl that may be covalently bonded to other functional groups by substituting the halogen with a nucleophilic functional group such as an amine, carboxylate anion, thiol anion, or alkoxide ion; (d) a dienophile, for example, that may undergo a Diels-Elder reaction with a maleimido group; (e) an aldehyde or a ketone capable of forming carbonyl derivatives such as an imine, a hydrazone, a semicarbazone or an oxime; (f) a sulfonyl halide that reacts with an amine to form a sulfoamide; (g) a thiol that may be converted to a disulfide or react with an acyl halide; (h) an amine that may be acylated, alkylated, or oxidized; (i) an alkene that may undergo reactions such as cycloaddition, acylation, and the Michael reaction; (j) an epoxide that may react with an amine or a hydroxyl compound; and (k) a phosphoramidite and other standard functional groups useful in nucleic acid reactions may be used. The reactive group may be appropriately selected so as not to participate in or interfere with the reactions necessary to synthesize the compounds defined herein.

If necessary, the reactive group may be protected from participating in the reaction by the presence of the protecting group.

The protecting group is introduced by chemically converting the reactive group to provide reaction selectivity to at least some of the reactive group during a continuous chemical or biological reaction process.

The protecting group includes a hydroxyl protecting group, an amino protecting group, a carbonyl protecting group, a carboxyl protecting group, a thiol protecting group, and a phosphate protecting group. Unless otherwise defined herein, as the protecting group, a functional group that may be introduced and removed as a specific reactive group, other than the above-described protecting groups, may be used.

For example, when the reactive group is a hydroxyl, the protecting group may be selected from an alkanoyl group such as acetyl, benzoyl, pivaloyl, chloroacetyl, trifluoroacetyl, and methoxyacetyl; an alkyloxycarbonyl such as benzyloxycarbonyl, ethyloxycarbonyl, methoxybenzyloxycarbonyl, tert-butyloxycarbonyl, diphenylmethyloxycarbonyl, and 2,2,2-trichloroethyloxycarbonyl; an aralkyl group such as benzyl, nitrobenzyl, methoxybenzyl, trityl, methoxytrityl, 4,4-dimethoxytrityl (DMT), and diphenylmethyl; an alkyl group such as t-butyl, methoxymethyl, methoxyethyl (MOE), methyl, 2,2,2-trichloroethyl, 2-trimethylsilylethyl, methylthiomethyl, benzyloxymethyl, 2-(trimethylsilyl)-ethoxymethyl, and 9-fluorenylmethyl; an ether group such as methoxymethyl ether (MOM), benzyl ether, tert-butyldimethylsilyl ether, p-methoxybenzyl ether (PMB), p-methoxyphenyl ether, 0-methoxyethoxymethyl ether (MEM), methylthiomethyl ether, trimethylsilyl ether, methyl ether, and ethoxyethyl ether; an ester group such as acetate ester, methanesulfonate ester, and tert-butyl carbonate; a silyl group such as tert-butyldiphenylsilyl, trimethylsilyl, and triisopropylsilyl; a sulfonyl group such as a methanesulfonyl group; allyl; tetrahydropyranyl (THP); tetrahydrofuran (THF); or the following groups.

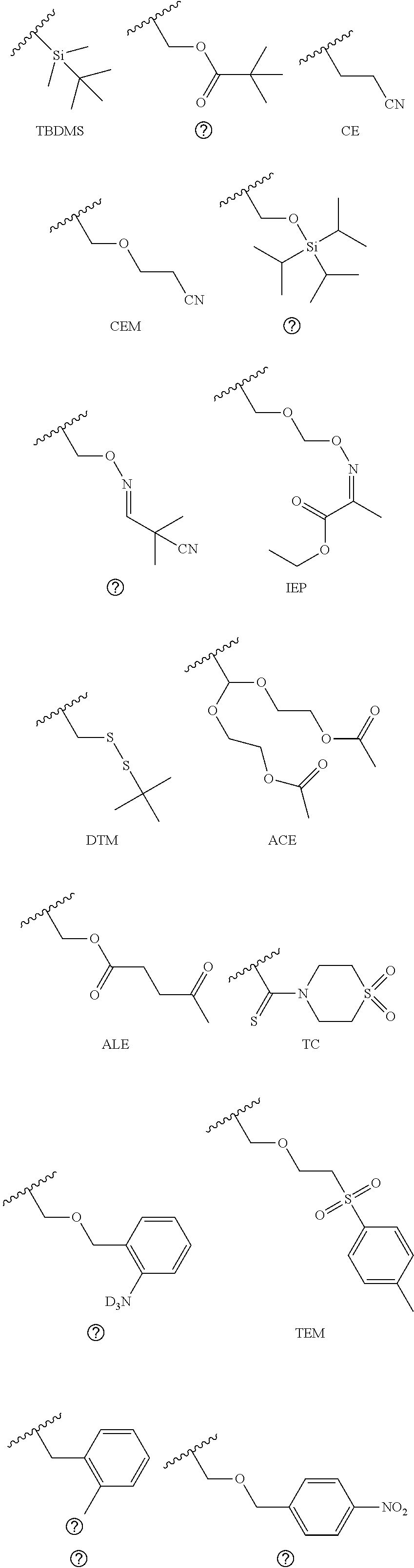

When the reactive group is amino, the protecting group may be selected from a carbamate group such as tert-butyl carbamate and benzyl carbamate; an amide group such as acetamide, p-toluenesulfonamide, and diphenyl-phospyramide; a phthalimide group; a carbonyl group such as 9-fluorenylmethoxycarbonyl (Fmoc), t-butyloxycarbonyl (Boc), t-amyloxycarbonyl, benzyloxycarbonyl (Cbz), 4-methoxybenzyloxycarbonyl, methyloxycarbonyl, ethyloxycarbonyl, nitrobenzyloxycarbonyl group, 2,2,2-trichloroethyloxycarbonyl group, aryloxycarbonyl, isobornyloxycarbonyl, and adamantyloxycarbonyl; a sulfonyl group such as 4-methylphenylsulfonyl, 2,4-dinitrobenzenesulfonyl, and 2-trimethylsilylethanesulfonyl (SES); an aralkyl group such as benzyl, trityl, 1,1-bis-(4-methoxyphenyl)methyl, 2,4-dimethoxybenzyl (DMB), and 4-methoxybenzyl (PMB); a silyl group such as trimethylsilyl (TMS) and t-butyldimethylsilyl (TBS); an alkanoyl group such as acetyl, trifluoroacetyl, phthalyl, pivaloyl, and benzoyl; allyl; 2-nitrophenylsulfenyl; or the following groups.

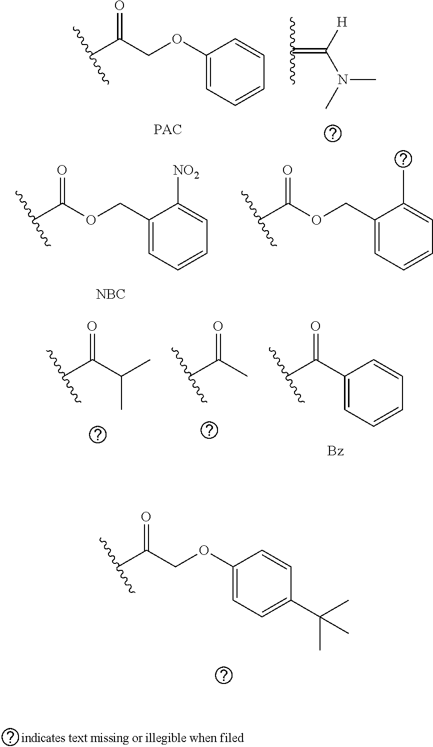

When the reactive group is carbonyl, as the protecting group, an acetal group such as dimethylacetal, 1,3-dioxane, and thioacetal; a ketal group such as dimethyl ketal and thioketal; an oxime such as O-methyloxime; a hydrazone such as N,N-dimethylhydrazone; or analogs thereof may be used.

When the reactive group is carboxyl, as the protecting group, an ester group such as methyl ester, allyl ester, and benzyl ester; an amide group such as N,N-dimethylamide; an alkyl group such as methyl, ethyl, t-butyl, phenylsulfonylethyl, cyanoethyl, 2-trimethylsilylethyl, 2-trimethylsilylethoxymethyl, and nitroethyl; an aralkyl group such as benzyl; an aryl group such as phenyl; an allyl group; or analogs thereof may be used.

When the reactive group is thiol, as the protecting group, an ether group such as benzylthioether; an ester group such as thioacetate ester, thiocarbonate, and thiocarbanate; or analogs thereof may be used.

When the reactive group is phosphoric acid, as the protecting group, an alkyl group such as t-butyl, methyl, ethyl, cyanoethyl, trimethylsilylethyl, triphenylsilylethyl, and 2,2,2-trichloroethyl; an alkenyl group such as ethenyl, propenyl, butenyl, 2-cyanobutenyl, and 1-ethyl-2-butenyl; a cycloalkyl group such as cyclopropyl, cyclobutyl, and cyclohexyl; an aralkyl group such as benzyl, α-naphthylmethyl, trityl, dimethylphenyl, chlorobenzyl, and nitrobenzyl; an aryl group such as phenyl, naphthyl, methylphenyl, dimethylphenyl, and chlorophenyl; an allyl group; or analogs thereof may be used.

As examples of the protecting group, reference may be made to: Greene's Protective Groups in Organic Synthesis, Third Edition. John Wiley & Sons, Inc. 1999; and https://en.wikipedia.org/wiki/Protecting_group.

The heteroatoms used herein may be atoms other than carbon and hydrogen, and more specifically, atoms that may be used to replace carbon in a backbone structure composed of carbon (more specifically, hydrocarbon), and for example, nitrogen, oxygen, sulfur, phosphorus, silicon, or selenium may be used.

When Rₓ herein (x is any integer selected from 1 to 11 or any letter selected from a to f) is an alkenyl or an alkynyl, the sp²-hybridized carbon of an alkenyl or the sp-hybridized carbon of an alkynyl may be bonded directly or indirectly bonded through the sp³-hybridized carbon of an alkyl bonded to the sp²-hybridized carbon of an alkenyl or the sp-hybridized carbon of an alkynyl.

In the present invention, the Cₐ-C_{b} functional group refers to a functional group having a to b carbon atoms. For example, Cₐ-C_{b} alkyl refers to a saturated aliphatic group, including a linear or branched alkyl having a to b carbon atoms. The linear or branched alkyl may have 40 or less carbon atoms in its main chain (such as C₁-C₁₀ linear or C₃-C₁₀ branched).

Specifically, the alkyl may be methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, n-heptyl, or n-octyl.

In addition, in the present invention, alkoxy refers to both an —O-(alkyl) group and an —O-(unsubstituted cycloalkyl) group, and is a linear or branched hydrocarbon having one or more ether groups and 1 to 10 carbon atoms.

Specific examples of the alkoxy include methoxy, ethoxy, propoxy, isopropoxy, n-butoxy, tert-butoxy, sec-butoxy, n-pentoxy, n-hexoxy, 1,2-dimethylbutoxy, cyclopropyloxy, cyclobutyloxy, cyclopentyloxy, and cyclohexyloxy, but the present invention is not limited thereto.

In the present invention, amino groups may be classified into primary to tertiary amino groups depending on the number of hydrogen atoms bonded to the nitrogen atom. If necessary, the amino groups may be provided in a quaternary ammonium form.

In the present invention, a halogen is fluoro (—F), chloro (—Cl), bromo (—Br), or iodo (—I), and a haloalkyl is an alkyl substituted with the above-described halogen. For example, a halomethyl is methyl in which at least one of the hydrogens is substituted with a halogen (—CH₂X, —CHX₂ or —CX₃).

In the present invention, “aralkyl” is the generic term for —(CH₂)ₙAr, which is a functional group in which a carbon of an alkyl is substituted with aryl. Examples of the aralkyl include benzyl (—CH₂C₆H₅) and phenethyl (—CH₂CH₂C₆H₅).

In the present invention, unless otherwise defined, aryl is an unsaturated aromatic ring including a single ring, or multiple rings (preferably, 1 to 4 rings) conjugated or connected by covalent bonds. Non-limiting examples of the aryl include phenyl, biphenyl, o-terphenyl, m-terphenyl, p-terphenyl, 1-naphthyl, 2-naphthyl, 1-anthryl, 2-anthryl, 9-anthryl, 1-phenanthrenyl, 2-phenanthrenyl, 3-phenanthrenyl, 4-phenanthrenyl, 9-phenanthrenyl, 1-pyrenyl, 2-pyrenyl, and 4-pyrenyl.

In the present invention, heteroaryl is a functional group in which one or more carbon atoms in the aryl defined above are substituted with a non-carbon atom such as nitrogen, oxygen or sulfur. Non-limiting examples of the heteroaryl include furyl, tetrahydrofuryl, pyrrolyl, pyrrolidinyl, thienyl, tetrahydrothienyl, oxazolyl, isoxazolyl, triazolyl, thiazolyl, isothiazolyl, pyrazolyl, pyrazolidinyl, oxadiazolyl, thiadiazolyl, imidazolyl, imidazolinyl, pyridyl, pyridaziyl, triazinyl, piperidinyl, morpholinyl, thiomorpholinyl, pyrazinyl, piperainyl, pyrimidinyl, naphthyridinyl, benzofuranyl, benzothienyl, indolyl, indolinyl, indolizinyl, indazolyl, quinolizinyl, quinolinyl, isoquinolinyl, cinnolinyl, phthalazinyl, quinazolinyl, quinoxalinyl, pteridinyl, quinuclidinyl, carbazoyl, acridinyl, phenazinyl, phenothizinyl, phenoxazinyl, purinyl, benzimidazolyl, benzothiazolyl, and analogs conjugated therewith.

In the present invention, unless otherwise defined, a hydrocarbon ring (cycloalkyl) or a hydrocarbon ring having a heteroatom (heterocycloalkyl) may be understood as a cyclic structure of an alkyl or heteroalkyl, respectively.

Non-limiting examples of the cycloalkyls include cyclopentyl, cyclohexyl, 1-cyclohexenyl, 3-cyclohexenyl, and cycloheptyl. Non-limiting examples of the heterocycloalkyls include 1-(1,2,5,6-tetrahydropyrinyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothiene-2-yl, tetrahydrothiene-3-yl, 1-piperazinyl, and 2-piperazinyl.

In the present invention, unless otherwise defined, cycloalkene or cycloalkyne may be understood as a cyclic structure of an alkyl containing at least one unsaturated bond (double bond or triple bond). At least one carbon of the cycloalkene or cycloalkyne may be substituted with a heteroatom. The cycloalkene or cycloalkyne may have 3 to 30 carbon atoms or 3 to 20 carbon atoms.

A hydrocarbon ring or a hydrocarbon ring containing a heteroatom may have a hydrocarbon ring, a hydrocarbon ring containing a heteroatom, an aryl, or heteroaryl conjugated or covalently linked thereto.

Derivatives used herein refer to similar compounds obtained by chemically changing a part of any compound.

For example, carboxyl derivatives herein may be N-hydroxysuccinimide esters, pentafluorophenyl esters, tetrafluorophenyl esters, sulfotetrafluorophenyl esters, N-hydroxybenztriazole esters, acyl halides, acyl imidazoles, thioesters, p-nitrophenyl esters, alkyl esters, alkenyl esters, alkynyl esters, or aromatic esters.

In the present invention, 1,2,4,5-tetrazine derivatives may be 3,6-dimethyl-1,2,4,5-tetrazine, 3,6-diphenyl-1,2,4,5-tetrazine, or 3-methyl-6-phenyl-1,2,4,5-tetrazine.

In the present invention, cycloalkyne derivatives may be compounds that participate in a bipolar cycloaddition, inverse-electron demand Diels-Alder, or strain-promoted azide-alkyne cycloaddition (SPAAC) reaction.

For example, cycloalkyne derivatives participating in the SPAAC reaction may include a cyclooctyne such as OCT, COMBO (ALO), MOFO, DIFO, DIBO, BARAC, DIBAC (ADIBO), DIMAC, BCN, or TMTH.

Examples of any derivative specified herein may be found through references known in the relevant technical field.

Polyalkylene oxide is an aqueous polymer functional group such as polyethylene glycol (PEG), polypropylene glycol (PPG), polyethylene glycol-polypropylene glycol (PEG-PPG) copolymers, and N-substituted methacrylamide-containing polymers and copolymers.

Polyalkylene oxide may be additionally substituted as needed, as long as the properties of the polymer are maintained. For example, the substitution may be a chemical bond to increase or decrease the chemical or biological stability of the polymer. As a specific example, any carbon or terminal carbon in the polyalkylene oxide may be substituted with hydroxy, alkyl ethers (such as methyl ether, ethyl ether, propyl ether, etc.), carboxymethyl ether, carboxyethyl ether, benzyl ether, dibenzylmethylene ether, or dimethylamine. In an embodiment, polyalkylene oxide may be polyalkylene oxide (mPEG) terminated with a methyl ether, where mPEG is represented by the chemical formula —(CH₂CH₂O)ₙCH₃, and the size of mPEG may vary depending on the size of n, which corresponds to the number of ethylene glycol repeating units. n, which corresponds to the number of ethylene glycol repeating units, may be an integer from 1 to 30.

The compounds represented by Chemical Formulas 1 to 8 may further include counterions. The counterion is an organic or inorganic anion, and may be appropriately selected considering the solubility and stability of the compounds.

Examples of the counterions of a reporter according to an embodiment of the present invention may include inorganic acid anions such as phosphoric acid hexafluoride ions, halogen ions, phosphoric acid ions, perchlorate ions, periodate ions, antimony hexafluoride ions, tartaric acid hexafluoride ions, fluoroboric acid ions, and tetrafluoride ions, and organic acid ions such as thiocyanate ions, benzenesulfonic acid ions, naphthalenesulfonic acid ions, p-toluenesulfonic acid ions, alkylsulfonic acid ions, benzenecarboxylic acid ions, alkylcarboxylic acid ions, trihaloalkylcarboxylic acid ions, alkylsulfonic acid ions, trihaloalkylsulfonic acid ions, and nicotinic acid ions. Metal compound ions such as bisphenylditol, thiobisphenol chelate, and bisdiol-α-diketone, metal ions such as sodium and potassium, and quaternary ammonium salts may also be selected as counterions.

Specific examples of the compounds defined herein are as follows. However, the following exemplary compounds are intended to aid understanding of the compounds defined herein and are not intended to limit the scope of the compound defined herein. All of the following exemplary compounds and compounds recognized as equivalent to the following exemplary compounds according to the scope of the compounds defined herein may be expected to exhibit the effects of the compounds defined herein. In addition, except for Compound 2, Compound 5, Compound 6, Compound 7, Compound 11, Compound 12, Compound 13, Compound 50, and Compound 51, the remaining exemplary compounds whose synthesis steps are described in detail through the Preparation Examples disclosed herein may also be synthesized with reference to the Preparation Examples disclosed herein, or through known synthesis methods with reference to the content defined herein.

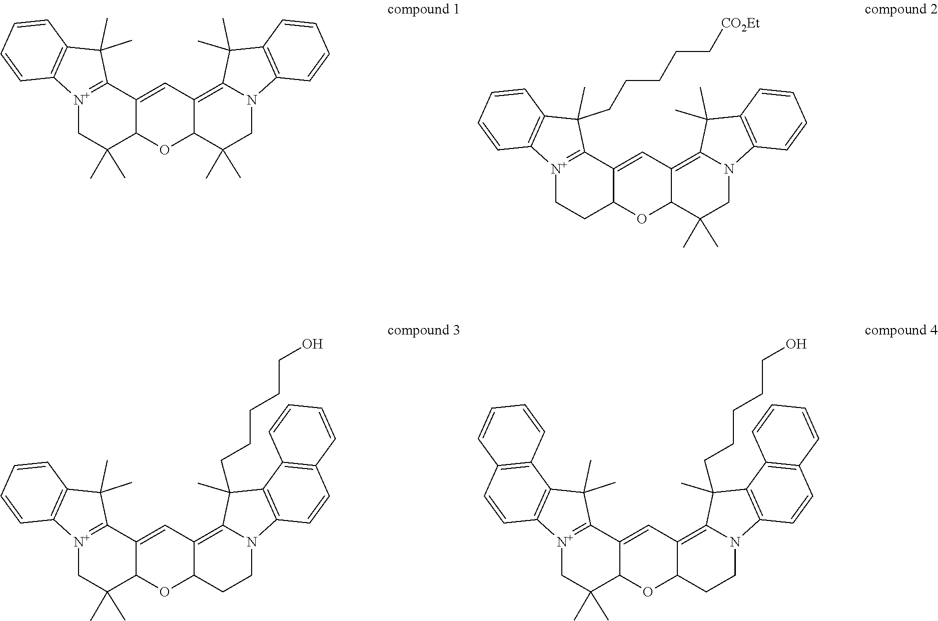 [SMILES: CC1(C)C2=C3C=C4C5=[N+](CC(C)(C)C4OC3C(C)(C)CN2c2ccccc21)c1ccccc1C5(C)C ; CCOC(=O)CCCCCC1(C)C2=[N+](CCC3OC4C(=C5N(CC4(C)C)c4ccccc4C5(C)C)C=C23)c2ccccc21 ; CC1(C)C2=[N+](CC(C)(C)C3OC4CCN5C(=C4C=C23)C(C)(CCCCCO)c2c5ccc3ccccc23)c2ccccc21 ; CC1(C)C2=[N+](CC(C)(C)C3OC4CCN5C(=C4C=C23)C(C)(CCCCCO)c2c5ccc3ccccc23)c2ccc3ccccc3c21]

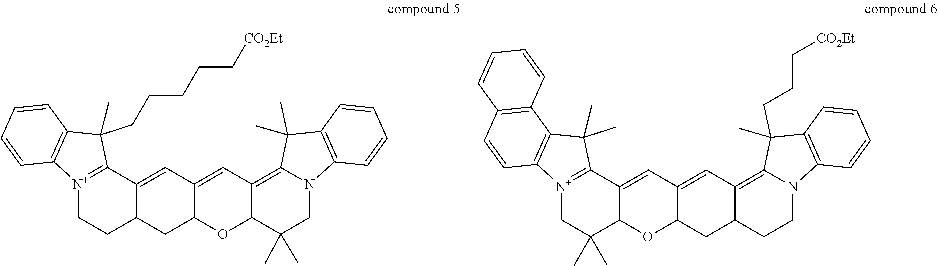 [SMILES: CCOC(=O)CCCCCC1(C)C2=[N+](CCC3CC4OC5C(=C6N(CC5(C)C)c5ccccc5C6(C)C)C=C4C=C23)c2ccccc21 ; CCOC(=O)CCCC1(C)C2=C3C=C4C=C5C6=[N+](CC(C)(C)C5OC4CC3CCN2c2ccccc21)c1ccc2ccccc2c1C6(C)C]

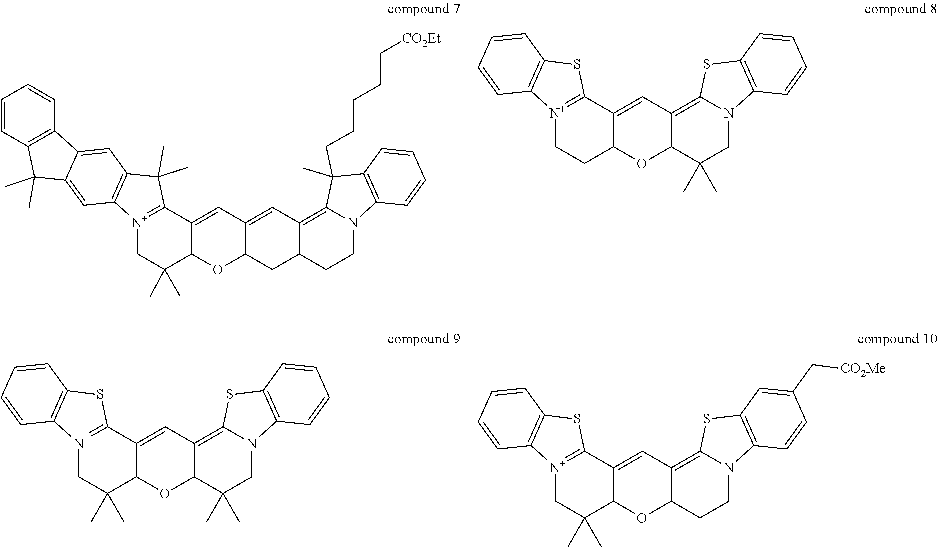

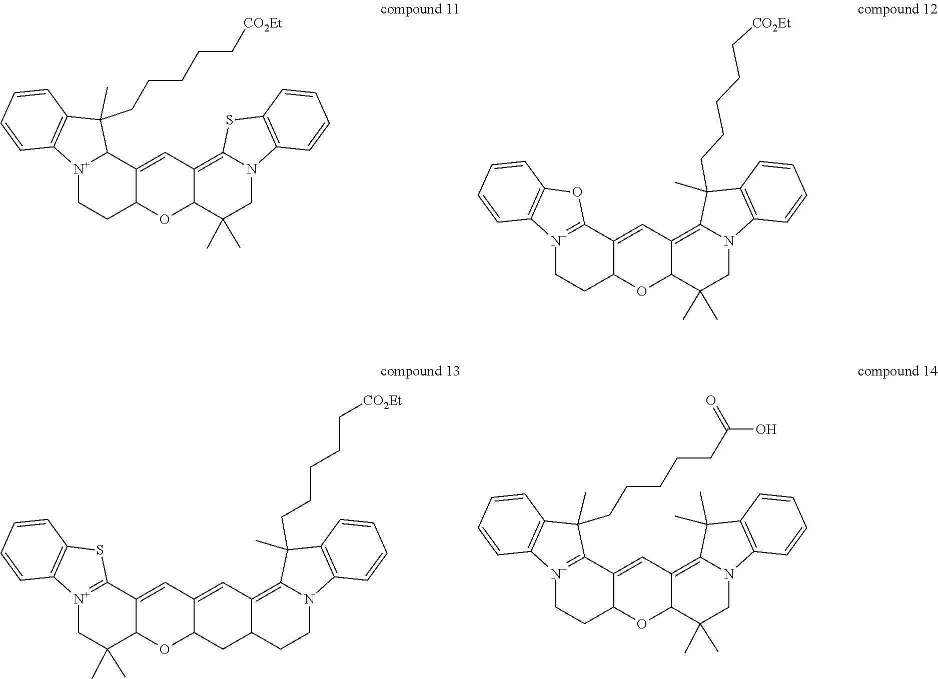

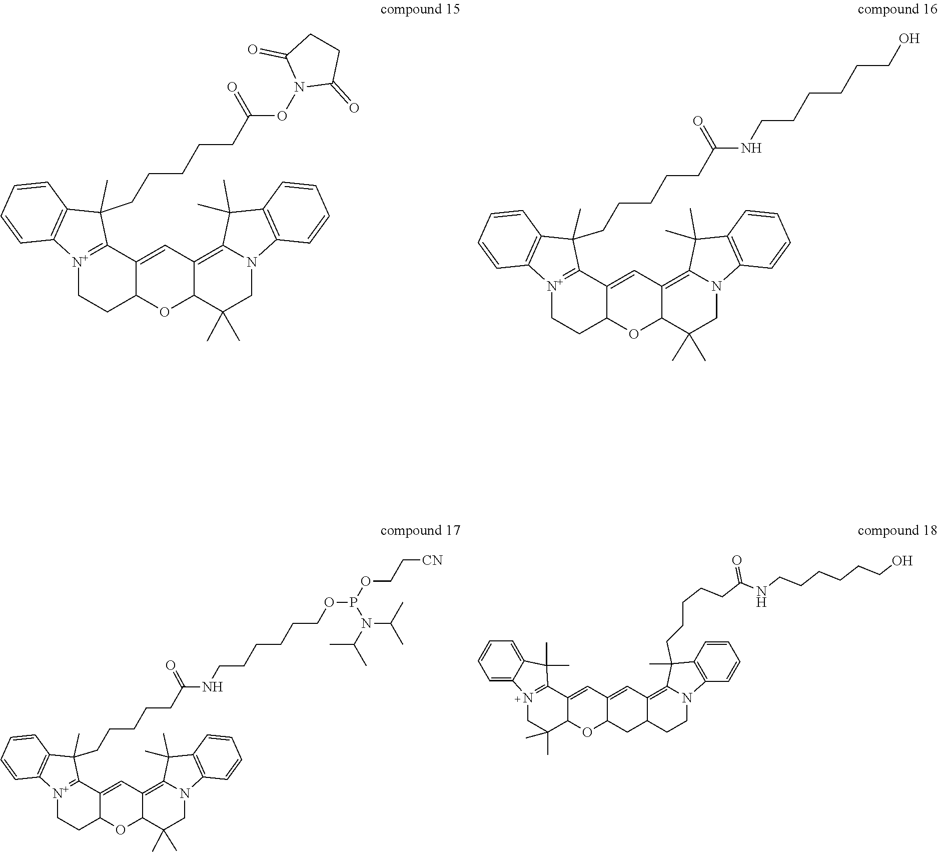

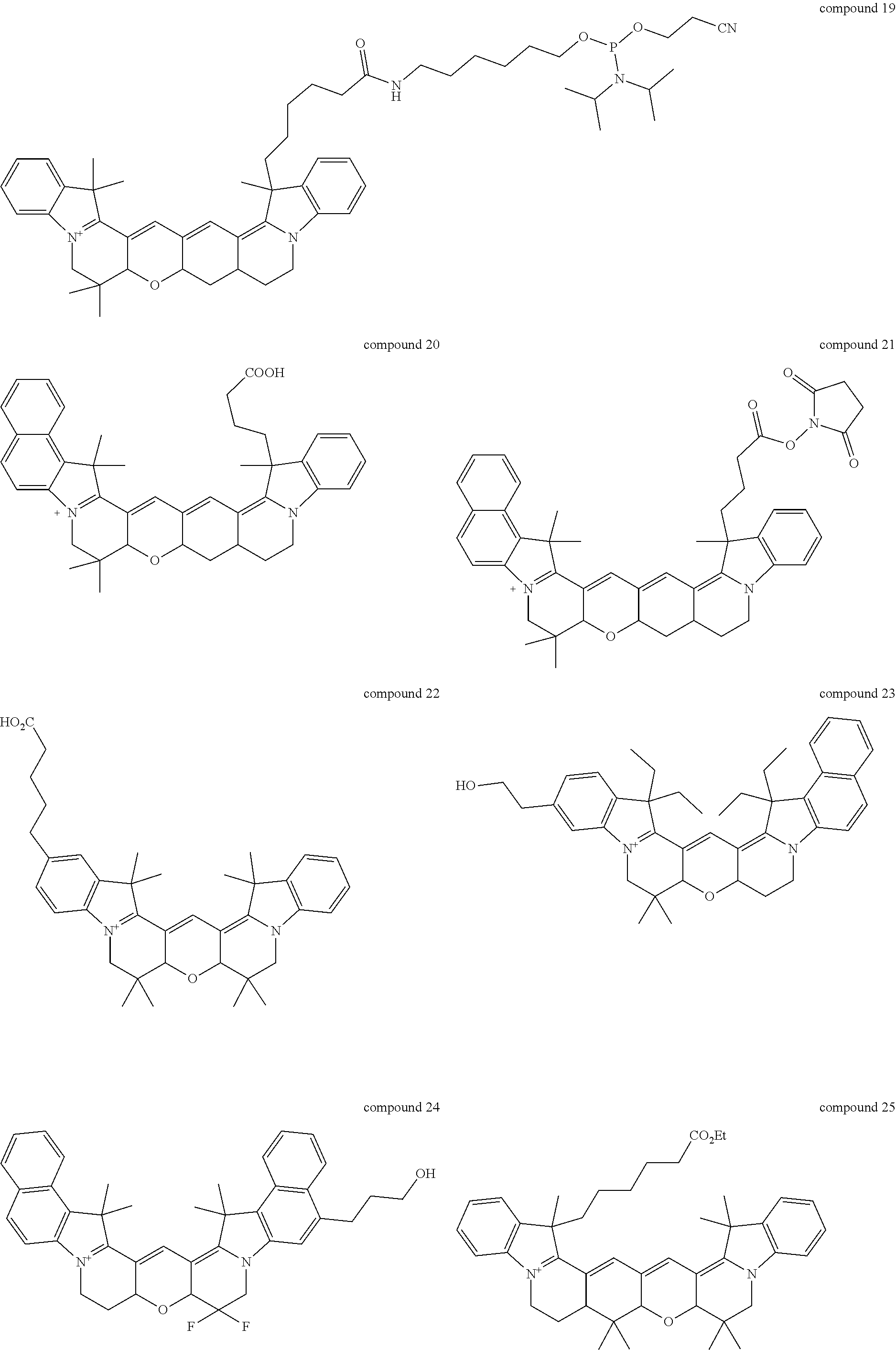

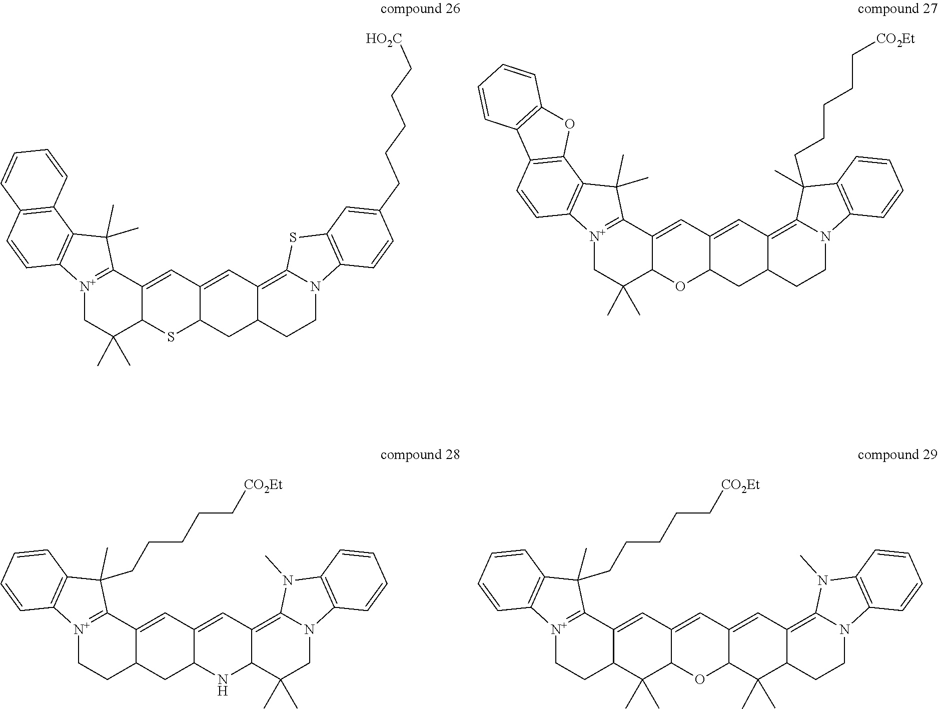

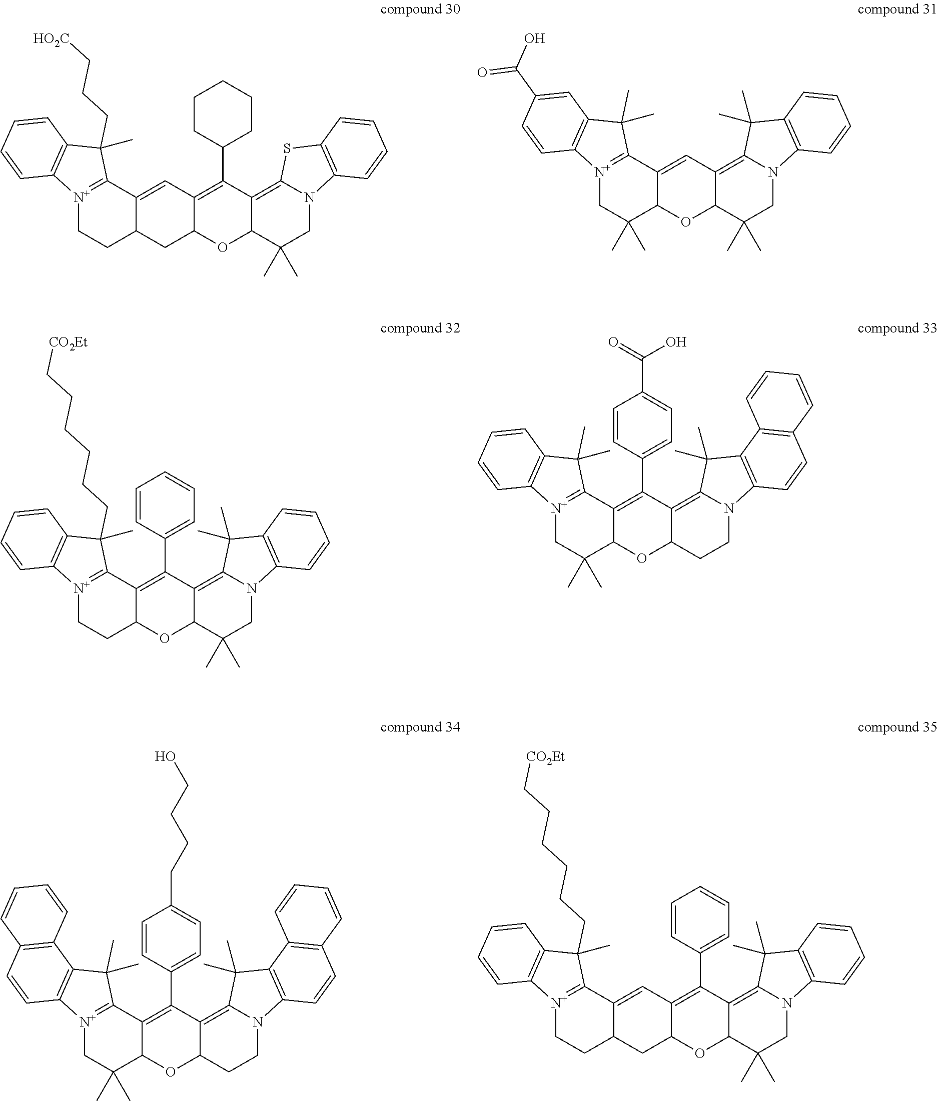

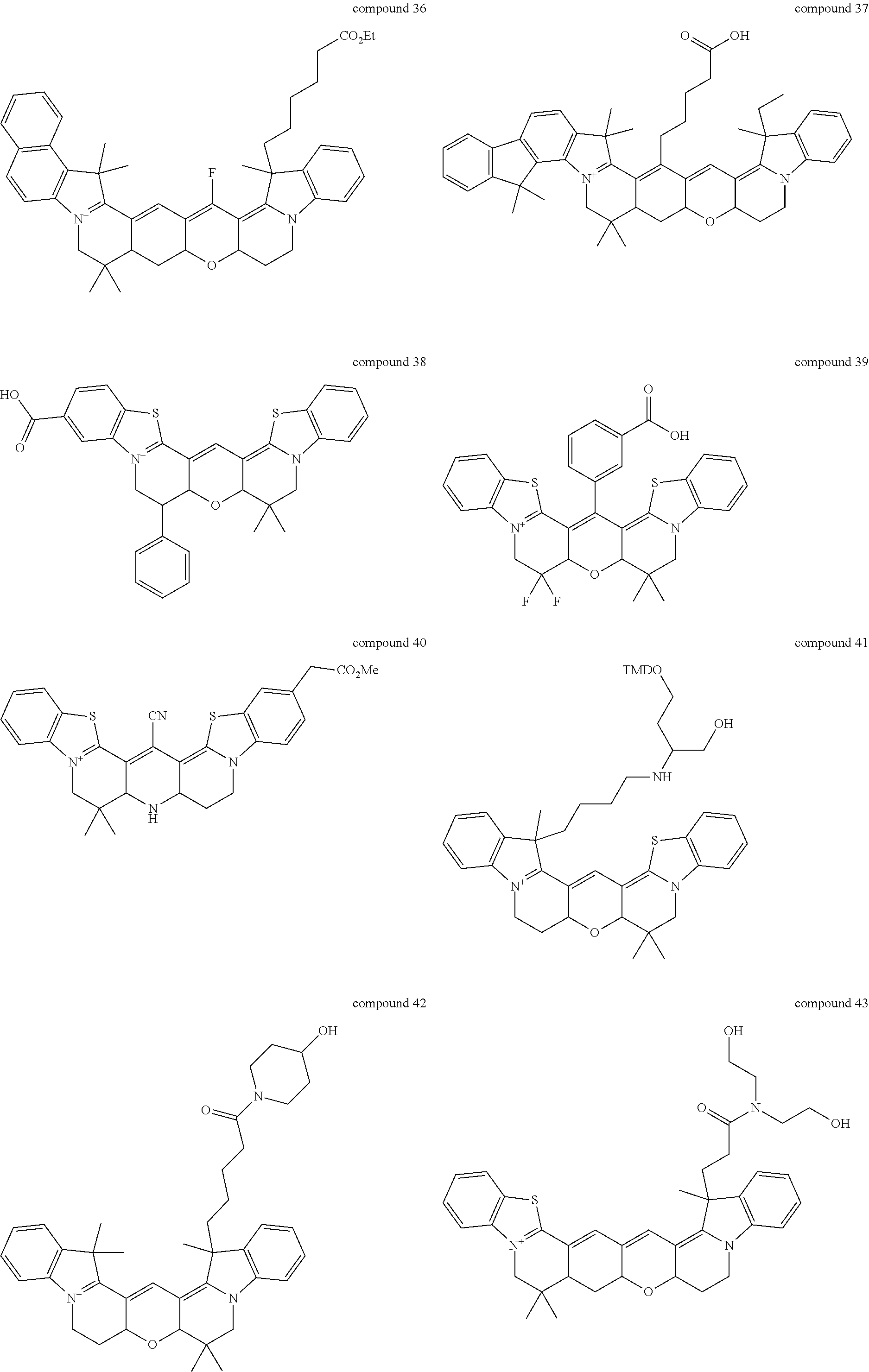

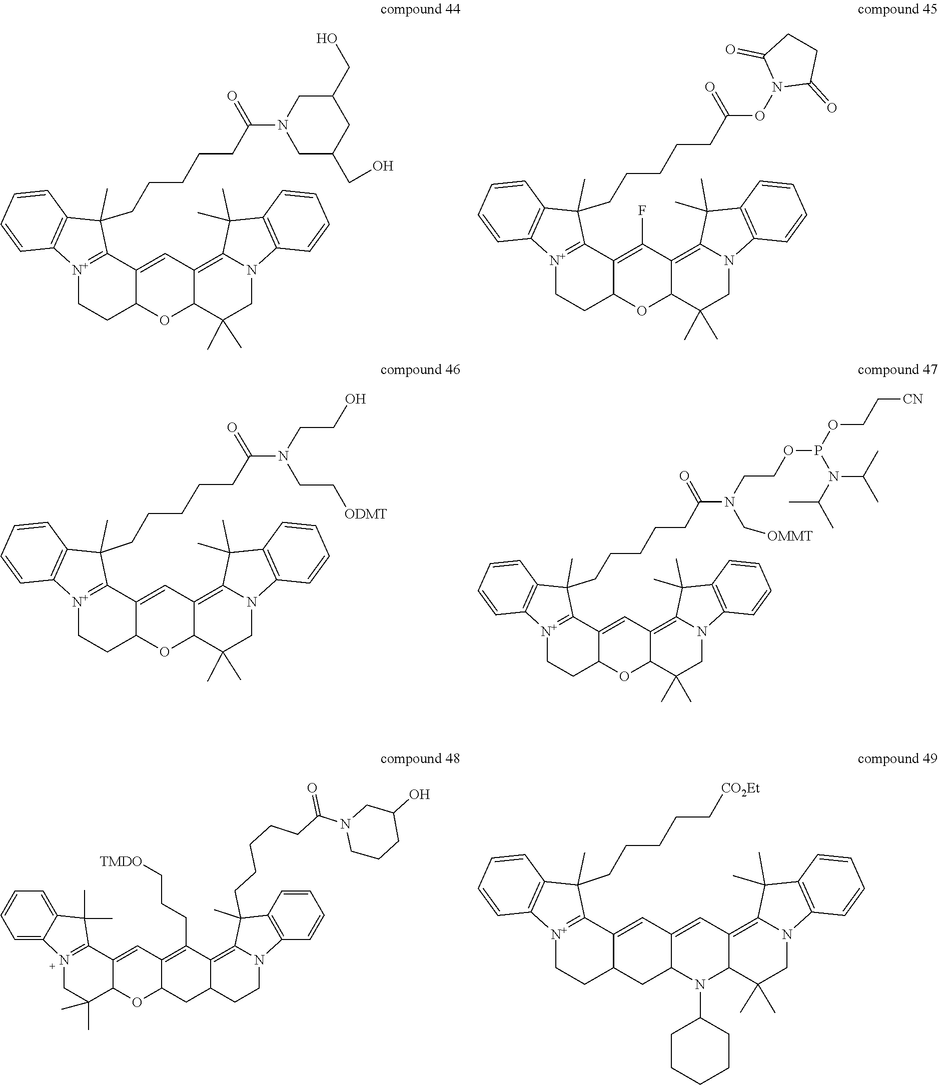

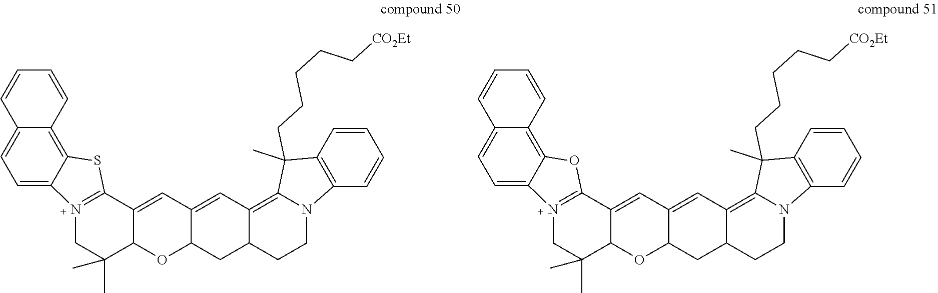

The compounds defined herein may be used to label or detect biomolecules. The biomolecules include antibodies, lipids, proteins, peptides, carbohydrates, and/or nucleic acids (including DNA, RNA, or nucleotides).

Specific examples of lipids include fatty acids, phospholipids, and lipopolysaccharides, and specific examples of carbohydrates include monosaccharides, disaccharides, and polysaccharides (such as dextrans).

The compounds defined herein may be used to label or detect not only biomolecules, but also drugs, hormones (including receptor ligands), receptors, enzymes or enzyme substrates, cells, cell membranes, toxins, microorganisms, or nano-bio materials (such as polystyrene microspheres), which contain at least one selected from amino, sulphydryl, carbonyl, hydroxyl, carboxyl, phosphate, and thiophosphate.

### Oligonucleotide Containing Novel Compound, Composition for Detecting Nucleic Acid, Support for Detecting Nucleic Acid

According to another aspect of the invention, there is provided an oligonucleotide containing a compound defined herein as a reporter for labeling a nucleic acid.

Oligonucleotide refers to a polymer of one to hundreds of nucleotides, and includes DNA, RNA, or PNA. It includes those that may be easily modified by a person skilled in the art, such as analogs thereof, for example, those in which chemical modifications have been made to the nucleotides or those in which sugars are linked, and those that are single-stranded or double-stranded ones.

The oligonucleotide preferably includes a probe. Such a probe is more preferably a probe that is capable of complementarily binding to a target nucleic acid, but the present invention is not limited thereto. The probe may be selected from a nucleic acid, a peptide, a saccharide, an oligonucleotide, a protein, an antibody, or a combination thereof, but the present invention is not limited thereto.

In one embodiment, the oligonucleotide may include a quencher. For example, the 5′ end of the oligonucleotide may be labeled with the reporter represented by Chemical Formula 1, 2, 5, or 6, and the 3′ end thereof may be labeled with the quencher. A probe capable of complementarily binding to a target nucleic acid may be located between the 5′ end and the 3′ end.

The maximum absorbance of the quencher usable in the present invention may be 620 to 700 nm, and preferably, 660 to 680 nm, and the absorbance range of the quencher may be 530 to 730 nm. In addition, the maximum absorbance and absorbance range of the quencher may be appropriately selected considering the fluorescence properties of the reporter defined herein.

It is important that the probe is designed such that the reporter can be sufficiently quenched by the quencher while minimizing signal crosstalk. Accordingly, when designing a probe, depending on the type of target biomolecule (such as a nucleic acid), it is necessary to confirm that the reporter and the quencher, which are labeled at the 5′ end and the 3′ end of the probe, respectively, are compatible with each other.

As the quencher, various known or commercially available quenchers (such as BHQ0, BHQ1, BHQ2, BHQ3, BBQ650, DABCYL, TAMRA, MGBEclipse, Atto540Q, Atto575Q, Atto612Q, QSY7, and QSY21) may be used. In addition, as the quencher, the quenchers disclosed in Korean Unexamined Patent Application Publication No. 10-2020-0067733 may be used. Representative examples of the quenchers disclosed in Korean Unexamined Patent Publication No. 10-2020-0067733 are as follows.

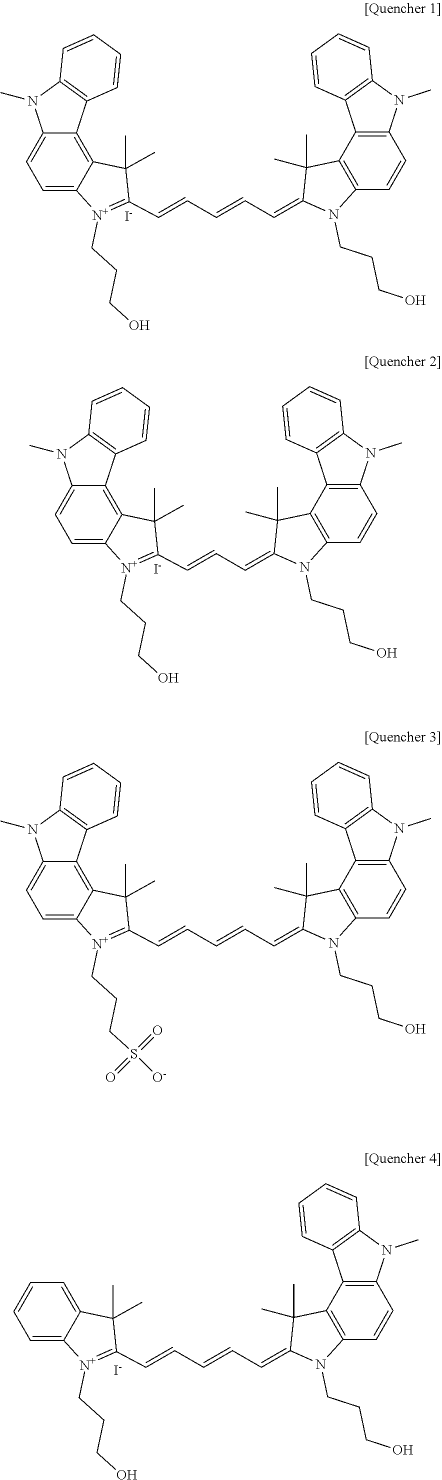

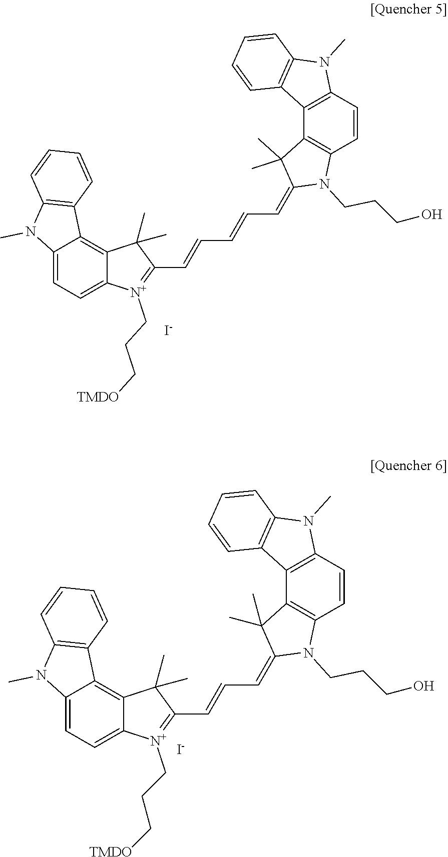

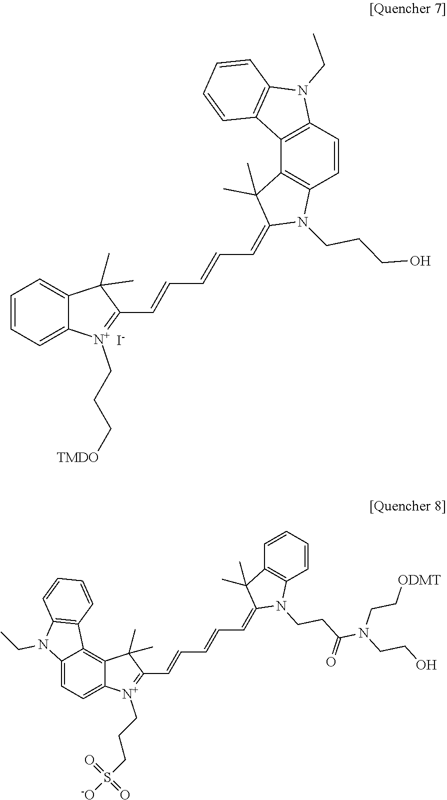

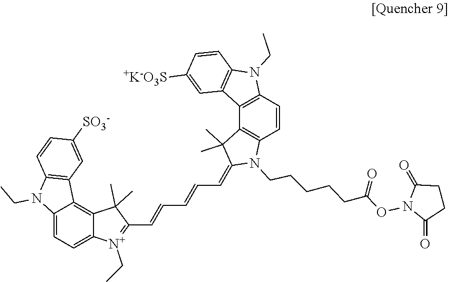

In addition, the oligonucleotide according to the present invention may further include a minor groove binder (MGB) to improve the binding strength to a nucleic acid.

The MGB is a crescent-shaped probe that can selectively bind non-covalently to a minor groove (e.g., shallow furrow in the DNA helix) included in a nucleic acid such as DNA.

Such an oligonucleotide may be used in various ways in the fields of chemistry and biology. Particularly, it may be useful for real time PCR or a microassay, but the present invention is not limited thereto.

In addition, according to another aspect of the present invention, a composition for detecting a nucleic acid, including the oligonucleotide, is provided.

The composition for detecting a nucleic acid according to one embodiment of the present invention may further include an enzyme, a solvent (buffer, etc.) and other reagents, which are used for a reaction with a target biomolecule, in addition to an oligonucleotide including a compound defined herein and a quencher at the same time.

Wherein, as the solvent, a buffer selected from the group consisting of a phosphate buffer, a carbonate buffer and a Tris buffer, an organic solvent selected from dimethyl sulfoxide, dimethylformamide, dichloromethane, methanol, ethanol and acetonitrile, or water may be used, and it is possible to adjust solubility by introducing various functional groups to a compound defined herein according to the type of solvent.

In addition, according to still another aspect of the present invention, a support for detecting a nucleic acid, which includes a probe singly labeled with a compound defined herein or dual-labeled probe with a compound defined herein and the quencher, a support, and a linker that connects the probe and the support, is provided.

Accordingly, a biomolecule in a sample may be fixed on a support matrix through interaction with the probe fixed on the support.

When the probe bound to the linker is a dual-labeled probe, the dual-labeled probe may be labeled with a compound defined herein at the 5′ end, labeled with a quencher at the 3′ end, and have a probe capable of complementary binding to the target nucleic acid located between the 5′ end and the 3′ end.

The support may be made of at least one selected from glass (such as controlled pore glass (CPG)), cellulose, nylon, acrylamide gel, dextran, polystyrene, a resin, alginate, collagen, a peptide, fibrin, hyaluronic acid, agarose, polyhydroxyethyl methacrylate, polyvinyl alcohol, polyethylene glycol, polyethylene oxide, polyethylene glycol diacrylate, gelatin, Matrigel, polylactic acid, carboxymethyl cellulose, dextran, chitosan, latex, and Sepharose, but is not limited thereto. The support may be in the form of beads or a membrane.

The linker is a part which connects the reporter and the support, and any material capable of connecting the reporter and the support may be used as a linker intended by the present invention.

For example, the linker may be selected from a substituted or unsubstituted C₁-C₃₀ alkyl, a substituted or unsubstituted C₃-C₃₀ cycloalkyl, a substituted or unsubstituted C₂-C₃₀ heteroalkyl containing at least one heteroatom, a substituted or unsubstituted C₂-C₃₀ heterocycloalkyl containing at least one heteroatom, a substituted or unsubstituted C₂-C₃₀ alkenyl, a substituted or unsubstituted C₆-C₃₀ aryl, a substituted or unsubstituted C₃-C₃₀ heteroaryl, amide (—CONH—), ester (—COO—), ketone (—CO—), nucleosides, and any combination thereof. The linker only connects a reporter and a support and does not affect other reactions or the fluorescence and quenching actions of the reporter or quencher.

Examples of the connection structure between the support and the quencher via the linker are as follows.

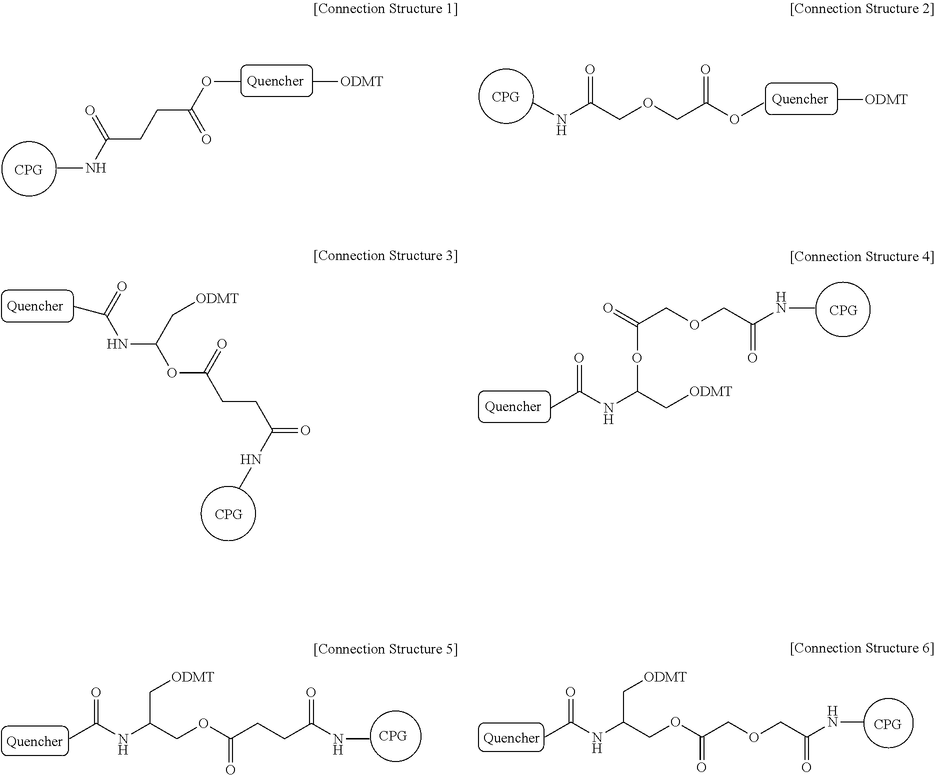

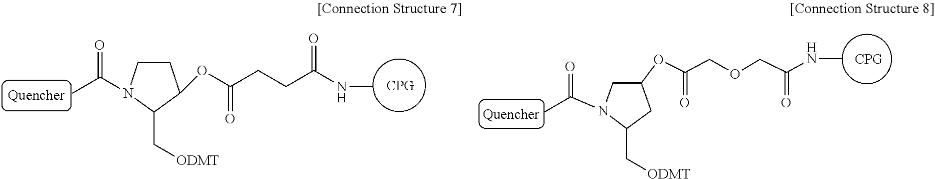

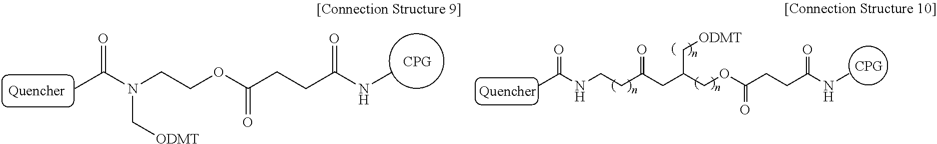

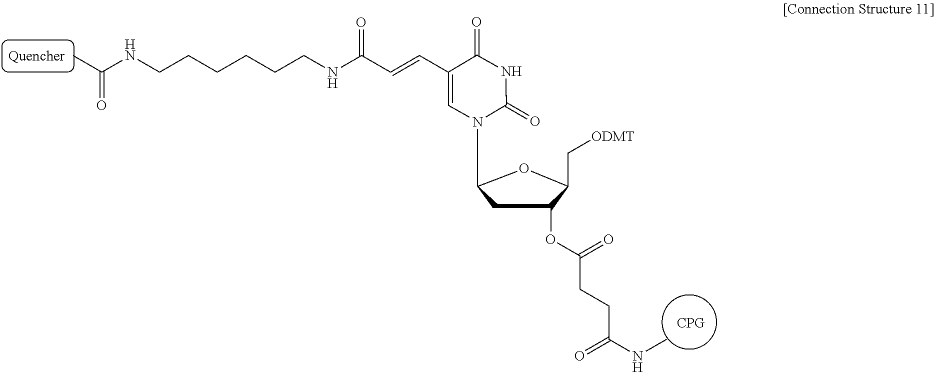

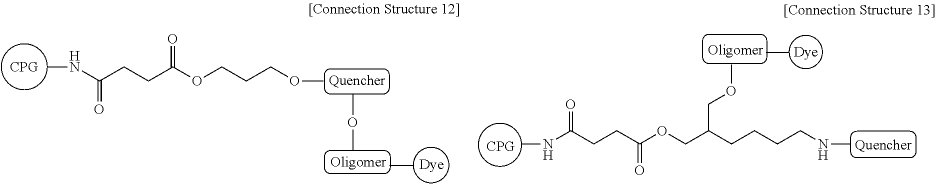

### Method of Detecting Nucleic Acid

According to another aspect of the present invention, there is provided a method of detecting a nucleic acid, including: (a) preparing a reaction mixture including a target nucleic acid, a reagent required for amplifying the target nucleic acid, and the nucleotide conjugate defined herein; (b) amplifying the target nucleic acid in the reaction mixture; and (c) measuring the fluorescence intensity of the reaction mixture.

Step (b) may include: (b-1) elongating the nucleotide conjugate hybridized to the target nucleic acid by a polymerase; (b-2) separating the reporter of the nucleotide conjugate and the quencher from the target nucleic acid by the exonuclease activity of the polymerase; and (b-3) allowing the reporter separated from the quencher to emit fluorescence.

Step (b) may be performed by a method selected from strand displacement amplification (SDA), polymerase chain reaction (PCR), reverse transcription polymerase chain reaction (RT-PCR), real-time polymerase chain reaction, allele-specific polymerase chain reaction, ligase chain reaction (LCR), rolling circle amplification (RCA), isothermal multiple displacement amplification (IMDA), recombinase polymerase amplification (RPA), self-sustained sequence replication (3SR), single primer isothermal amplification (SPIA), multiple displacement amplification (MDA), whole genome amplification (WGA), cross-priming amplification (CPA), signal mediated amplification of RNA technology (SMART), transcription mediated amplification (TMA), nucleic acid sequence based amplification (NASBA), loop-mediated isothermal amplification (LAMP), and helicase dependent amplification (HDA).

The method may further include (d) measuring an amplification amount of the target nucleic acid from the fluorescence intensity measured in (c).

### DNA Microarray

A DNA microarray is for measuring the fluorescence of a target nucleic acid by preparing a single-stranded probe nucleic acid which labels a target nucleic acid through a reaction with a dye and has a complementary base sequence to the target nucleic acid, and hybridizing the probe nucleic acid with the target nucleic acid denatured into a single strand on a substrate.

In the labeling method, when gene expression is investigated, as the probe nucleic acid immobilized on the substrate, cDNA, which is prepared by amplifying a cDNA library, genome library, or any of all genomes as a template through PCR, may be used.

In addition, for investigation of gene mutations, various oligonucleotides corresponding to mutations may be synthesized based on a known sequence serving as a reference and used.

A proper method for immobilizing the probe nucleic acid on the substrate may be selected according to the type of nucleic acid or substrate. For example, a method for electrostatic binding to a substrate surface-treated with a cation such as polylysine using the charge of DNA may also be used.

The target nucleic acid denatured into a single strand is immobilized on the substrate, and hybridized with the oligonucleotide. Wherein, the 5′ end of the oligonucleotide is labeled with a compound defined herein, and the 3′ end thereof is labeled with a quencher. Between the 5′ end and the 3′ end, a probe that is able to complimentarily bind to the target nucleic acid may be located.

Hybridization is preferably performed at room temperature to 70° C. for approximately 2 to 48 hours. Through hybridization, a target nucleic acid having a complementary base sequence with the probe nucleic acid is selectively bound to probe nucleic acid. Afterward, the substrate is washed and dried at room temperature.

Wherein, the oligonucleotide is hybridized to the target nucleic acid by the probe, but the fluorophore at the 5′ end is present in a quenched state by the quencher at the 3′ end.

Subsequently, the oligonucleotide hybridized to the target nucleic acid is elongated by a polymerase, separated from the target nucleic acid due to the exonuclease activity of the polymerase, and degraded. The fluorophore at the 5′ end of the oligonucleotide and the quencher at the 3′ end thereof are separated from each other, and thus the fluorophore may exhibit fluorescence.

Wherein, the intensity of the generated fluorescence is measured to measure the amplification amount of the target nucleic acid.

### PCR Method

According to a PCR method, a probe complementary to the base sequence of a target nucleic acid to be labeled is labeled with a compound defined herein, and reacted with the target nucleic acid before or after the amplification of the target nucleic acid, and then the fluorescence of the target nucleic acid is measured.

Specifically, the elongation reaction of the target nucleic acid is carried out by an enzyme (DNA polymerase or RNA polymerase), and a double-stranded nucleic acid sequence formed of the target nucleic acid and a primer consisting of an oligonucleotide is recognized by the enzyme to carry out the elongation reaction from the recognition site, and only a target gene area is amplified.

When synthesis is performed by the enzyme, the synthesis reaction is carried out using nucleotides (dNTP and NTP) as raw materials.

Wherein, by mixing common nucleotides (dNTP and NTP) with reporter-bearing nucleotides in an arbitrary ratio, a nucleic acid into which the equivalent amount of dye is introduced may be synthesized.

In addition, a nucleic acid to which a reporter is introduced may be synthesized by bonding the reporter after introducing nucleotides having an amino group in an arbitrary ratio by PCR.

When synthesis is performed by the enzyme, the synthesis reaction is carried out using nucleotides as raw materials, and when a material in which the 3′ OH of the nucleotide is substituted with H is used, a nucleic acid is no longer elongated, and at this point of time, the reaction ends.

This nucleotide, that is, dideoxynucleotide triphosphate (ddNTP) is called a terminator.

When a terminator is mixed with common nucleotides to synthesize a nucleic acid, the terminator is introduced with a certain probability to end the reaction, so nucleic acids of various lengths are synthesized.

When the above are separated by size through gel electrophoresis, DNA is lined up in order of length. Wherein, when labeled with a different reporter for each type of terminator base, at the end point (3′ end) of the synthesis reaction, a dependency on each base is observed, and by reading fluorescence information starting with the reporter attached to the terminator, base sequence information of the target nucleic acid may be obtained.

In addition, instead of the terminator, primers previously labeled with the reporter may be used for hybridization with a target nucleic acid.

In addition, as a probe, a peptide nucleic acid (PNA) may also be used. PNA is obtained by replacing the pentose phosphate backbone, which is the basic skeleton of a nucleic acid, with a polyamide backbone composed of glycine as a unit, and PNA has a 3D structure highly similar to nucleic acids, and is very specific for a nucleic acid having a complementary base sequence and strongly binds thereto. Accordingly, PNA may also be used as a reagent for telomere research by applying a telomere PNA probe, in addition to a conventional DNA analysis method such as in-situ hybridization (ISH).

For labeling, for example, double-stranded DNA is brought into contact with a PNA having a base sequence complementary to all or a part of the base sequence of the DNA and labeled with a reporter for hybridization, the mixture is heated to generate single-stranded DNA, and slowly cooled to room temperature to prepare a PNA-DNA complex, and then fluorescence is measured.

In the above example, a method of amplifying a target nucleic acid through PCR and measuring the fluorescence of a product has been described, but in this method, it is necessary to identify the size of the product through electrophoresis and then investigate the amount of amplification product by measuring fluorescence intensity.

To this end, the amount of product may be measured in real time using the energy transfer of a fluorescent dye and a probe designed to generate fluorescence by hybridizing it to the PCR product.

For example, DNA labeled with a donor and an acceptor may be used. A specific labeling method may be a molecular beacon method, a TaqMan-PCR method, or a cycling probe method, which is used to confirm the presence of a nucleic acid having a specific sequence.

### Other Labeling Methods

In addition, the compound of the present invention may also be used as reporter in a method of labeling a target using specific binding.

That is, in the labeling of a sample including a target or a sample modified by a modifying material, one of a binding material specifically binding to the sample and a binding material specifically binding to the modifying material may be labeled with a reporter, and fluorescence may be measured from the labeled binding materials.

Wherein, for the combination of the sample or modifying material with the binding material, antigen-antibody, hapten-anti-hapten antibody, biotin-avidin, a Tag antigen, a Tag antibody, lectin-glycoprotein, or hormone-receptor may be used.

Specifically, a specific antigen may be labeled through antigen-specific interaction of an antibody by reacting a binding material such as a reporter-labeled antibody with an antigen present in a substrate, solution, beads, or an antibody.

An antigen may be a protein, a polysaccharide, a nucleic acid, or a peptide, and other than the antigen, a hapten such as a low-molecular-weight molecule, for example, FITC or a dinitrophenyl group may also be used. Wherein, as an antigen (or hapten)-antibody combination, there are GFP and anti GFP antibodies, FITC and anti-FITC antibodies and the like.

Labeled antigens may be used in various measurement methods including immunostaining, ELISA, Western blotting or flow cytometry.

In addition, an intracellular signaling phenomenon may be observed using the reporter of the present invention. Various enzymes are involved in internal signaling or cell responses according to the signaling. In a representative signaling phenomenon, it is known that a special protein kinase is activated, thereby inducing protein phosphorylation to initiate signaling.

Binding and hydrolysis of a nucleotide (e.g., ATP or ADP) play a critical role in its activity, and an intracellular signaling phenomenon may be observed with high sensitivity by introducing a reporter to a nucleotide derivative.

In addition, the reporter of the present invention may also be used in observation of a gene expression phenomenon using RNA interference (RNAi).

RNAi is inhibition of expression by degradation of mRNA of a target gene by introducing double-stranded RNA (dsRNA) into cells, and thus it is possible to observe the RNAi phenomenon by labeling designed dsRNA with a reporter.

In addition, since the reporter of the present invention has a reactive group capable of labeling a target nucleic acid or target protein in tissue or cells, it may be used as a dye for confirming the transcription level of a target nucleic acid or the expression level of a target protein.

Hereinafter, specific examples of the present invention are presented. However, the following examples are only for exemplifying or explaining the present invention in detail, and the present invention is not limited thereto. In addition, among the compounds defined in the claims and detailed description of the present invention, compounds whose synthesis methods are not disclosed through the following preparation examples may be synthesized with reference to the following preparation examples.

### Preparation Example 1. Synthesis of Compound 1

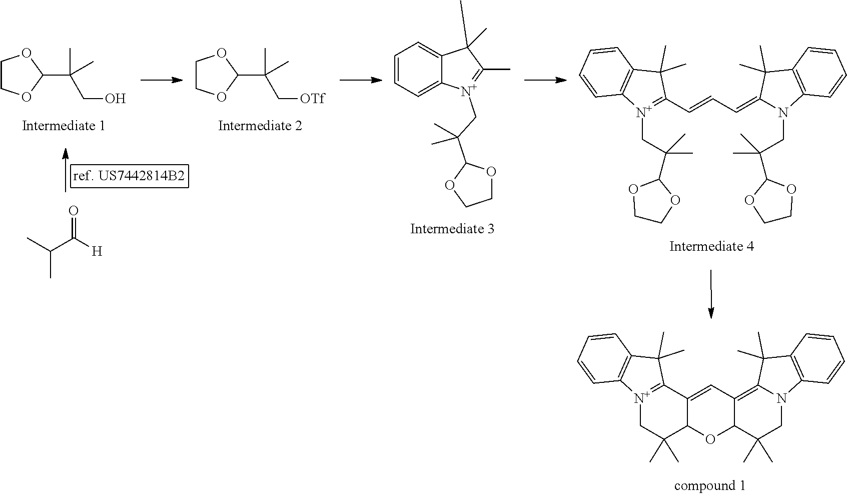

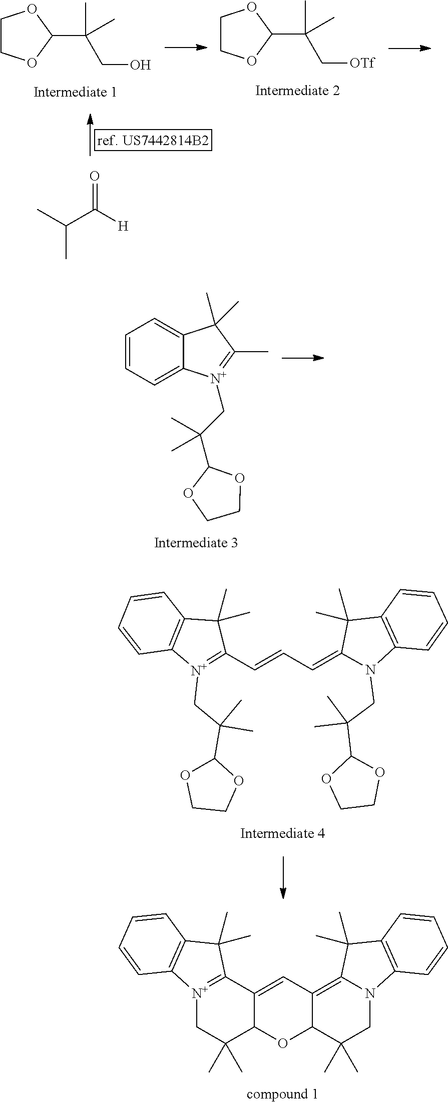

### Synthesis of Intermediate 2

Intermediate 1 (synthesized with reference to U.S. Pat. No. 7,442,814) (500 g, 3.42 mol), pyridine (360 mL, 4.45 mol), and dichloromethane (5000 mL) were added to a 10 L four-neck reactor and stirred at −78° C. Anhydrous trifluoromethanesulfonic acid (631 mL, 3.76 mol) was added dropwise and stirred at room temperature for 1 hour, and the resulting solid was removed by filtration. A 10% aqueous sodium bicarbonate solution (4000 mL) was added to the filtrate and stirred vigorously to separate the organic layer. After anhydrous sodium sulfate was added, stirred for 5 minutes and filtered, the filtrate was concentrated and column purified (930 g, 97%).

### Synthesis of Intermediate 3

Intermediate 2 (342 g, 1.23 mol), 2,3,3-trimerine indolenine (140 g, 0.88 mol), and dichloromethane (700 mL) were added to a 1 L one-neck reactor, stirred under reflux for 24 hours, cooled, and concentrated.

### Synthesis of Intermediate 4

Intermediate 3 (4 g, 9 mmol), triethylorthoformate (4.7 mL, 27 mmol), and pyridine (20 mL) were added to a 250 mL one-neck reactor, stirred under reflux for 3 hours, cooled, and concentrated.

### Synthesis of Compound 1

Intermediate 4 (5 g, 2.949 mmol), a 50% aqueous sulfuric acid solution (10 mL), and chloroform (50 mL) were added to a 250 mL one-neck reactor and stirred at room temperature for 24 hours. Water (30 mL) was added to the reactor and stirred vigorously to separate the organic layer, and then anhydrous sodium sulfate was added, stirred for 5 minutes, and filtered. The filtrate was concentrated, and column purified (20 mg).

The ¹H-NMR data of the obtained Compound 1 is as follows.

1H-NMR (400 MHz, CDCl3) δ 7.26 (m, 2H), 7.44-7.33 (m, 7H), 4.62 (s, 2H), 4.13-3.90 (m, 4H), 1.68 (s, 12H), 1.44 (s, 6H), 0.98 (s, 6H)

### Preparation Example 2. Synthesis of Compound 2 and Compounds 14 to 17

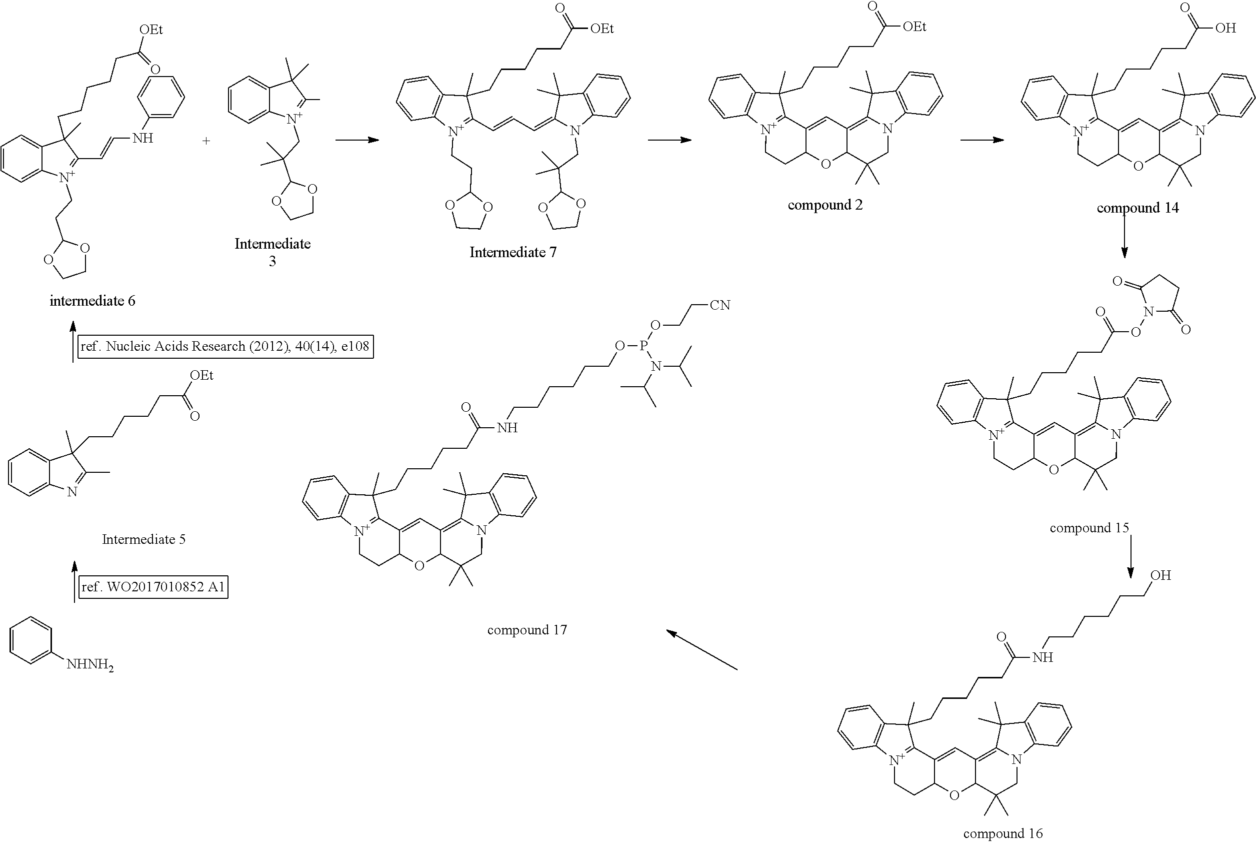

### Synthesis of Intermediate 7

Intermediate 6 (synthesized with reference to Nucleic Acid Research (2012), 40(14), e108) (98 g, 0.158 mmol), Intermediate 3 of Compound 1 (76.2 g, 0.174 mmol), acetic anhydride (160 mL), and pyridine (1000 mL) were added to a 3 Lone-neck reactor, stirred at 50° C. for 24 hours, concentrated, and column purified (80 g).

### Synthesis of Compound 2

Intermediate 7 (80 g, 0.098 mol), a 50% sulfuric acid aqueous solution (160 mL), and chloroform (800 mL) were added to a 2 L one-neck reactor and stirred at room temperature for 24 hours, and then water (500 mL) was added to the reactor and stirred vigorously to separate the organic layer. Anhydrous sodium sulfate was added, stirred for 5 minutes, and filtered, and the filtrate was concentrated, and column purified (26 g).

### Synthesis of Compound 14

Compound 2 (26.8 g, 0.038 mol), a 2N aqueous sodium hydroxide solution (70 mL), tetrahydrofuran (130 mL), and methanol (130 mL) were added to a 1 L one-neck reactor, stirred at room temperature for 24 hours, and concentrated, and then dichloromethane (400 mL) and 4 M hydrochloric acid (100 mL) were added to the reactor and stirred vigorously to separate the organic layer. Anhydrous sodium sulfate was added, stirred for 5 minutes, and filtered, and the filtrate was concentrated.

### Synthesis of Compound 15

Compound 14 (26.8 g, 0.038 mol), N,N′-dicyclohexylcarbodiimide (10.6 g, 0.051 mol), N-hydroxysuccinimide (5.9 g, 0.051 mol), and dichloromethane (300 mL) were added to a 1 L one-neck reactor and stirred at room temperature for 2 hours, and the resulting solid was removed by filtration. Brine (200 mL) was added to the filtrate and stirred vigorously to separate the organic layer. Afterward, anhydrous sodium sulfate was added, stirred for 5 minutes, and filtered, and the filtrate was concentrated.

### Synthesis of Compound 16

Compound 15 (48.5 g, 0.063 mol), 6-aminohexanol (22 g, 0.188 mol), and dichloromethane (500 mL) were added to a 1 L one-neck reactor and stirred at room temperature for 1 hour, and then water (200 mL) was added and stirred vigorously to separate the organic layer. Afterward, anhydrous sodium sulfate was added, stirred for 5 minutes, and filtered. The filtrate was concentrated, and column purified (43 g).

### Synthesis of Compound 17

Compound 16 (20.5 g, 0.028 mol), 2-cyanoethyl N,N′-diisopropylchlorophosphoramidite (8.6 g, 0.036 mol), triethylamine (8 mL, 0.051 mol), and dichloromethane (410 mL) were added to a 1 L one-neck reactor and stirred at room temperature for 1 hour. A 10% sodium carbonate solution (500 mL) was added to the reaction solution and stirred vigorously to separate the organic layer. Afterward, anhydrous sodium sulfate was added, stirred for 5 minutes, and filtered, and the filtrate was concentrated and column purified (21 g).

The ₁H-NMR data of the obtained compound 17 is as follows.

1H-NMR (400 MHz, CDCl3) δ 8.12 (m, 1H), 7.41-7.14 (m, 8H), 4.87 (m, 1H), 4.63-4.58 (m, 1H), 4.25-4.18 (m, 4H), 3.84-3.45 (m, 16H), 2.62-2.58 (m. 3H), 2.26-1.96 (m, 5H), 1.78-1.70 (m, 9H), 1.49-1.39 (m, 5H), 1.23-1.00 (m, 15H), 0.93 (s, 3H), 0.69 (m, 1H)

### Preparation Example 3. Synthesis of Compound 3

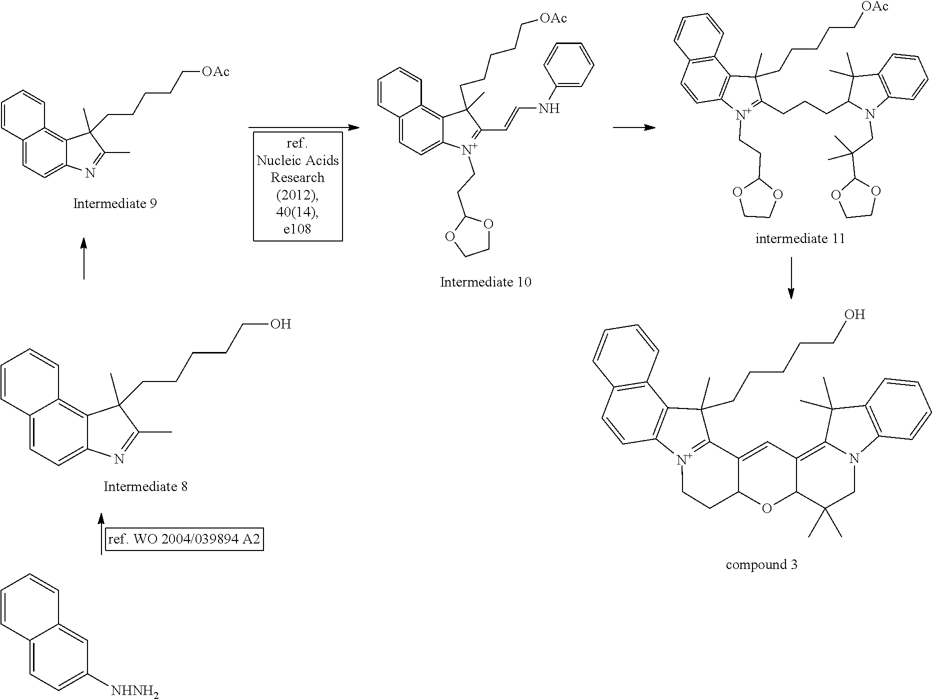 [SMILES: CC(=O)OCCCCCC1(C)C(C)=Nc2ccc3ccccc3c21 ; CC(=O)OCCCCCC1(C)C(/C=C/Nc2ccccc2)=[N+](CCC2OCCO2)c2ccc3ccccc3c21 ; CC(=O)OCCCCCC1(C)C(CCCC2N(CC(C)(C)C3OCCO3)c3ccccc3C2(C)C)=[N+](CCC2OCCO2)c2ccc3ccccc3c21 ; CC1=Nc2ccc3ccccc3c2C1(C)CCCCCO ; CC1(C)C2=C3C=C4C5=[N+](CCC4OC3C(C)(C)CN2c2ccccc21)c1ccc2ccccc2c1C5(C)CCCCCO ; NNc1ccc2ccccc2c1]

### Synthesis of Intermediate 9

Intermediate 8 (synthesized with reference to International Patent Publication No. 2004-039894) (2.78 g, 9.88 mmol), acetic anhydride (1.1 mL), triethylamine (2.8 mL), and dichloromethane (30 mL) were added to a 250 mL one-neck reactor and stirred at room temperature for 24 hours, and then water was added to the reaction solution and stirred vigorously to separate the organic layer. Afterward, anhydrous sodium sulfate was added, stirred for 5 minutes, and filtered, and the filtrate was concentrated, and column purified (2.3 g).

### Synthesis of Intermediate 10

Intermediate 10 was synthesized using Intermediate 9 with reference to Nucleic Acid Research (2012), 40(14), e108.

### Synthesis of Intermediate 11

Intermediate 10 (6 g, 9.17 mmol), Intermediate 3 of compound 1 (4 g, 9.17 mmol), acetic anhydride (9 mL), and pyridine (60 mL) were added to a 250 mL one-neck reactor and stirred at 50° C. for 24 hours. Afterward, the reaction solution was concentrated and column purified (4.5 g).

### Synthesis of Compound 3

Intermediate 11 (4.5 g, 5.30 mmol), a 50% aqueous sulfuric acid solution (10 mL), and chloroform (50 mL) were added to a 250 mL one-neck reactor and stirred at room temperature for 24 hours, and then water (100 mL) was added to the reactor and stirred vigorously to separate the organic layer. Afterward, anhydrous sodium sulfate was added, stirred for 5 minutes, and filtered, and the filtrate was concentrated, and column purified (1.2 g).

The ₁H-NMR data of the obtained Compound 3 is as follows.

1H-NMR (400 MHz, CDCl3) δ 8.29-8.25 (m, 1H), 8.10-8.07 (m, 1H), 7.98-7.95 (m, 2H), 7.61 (t, 1H, J=9.6 Hz), 7.52-7.44 (m, 2H), 7.42-7.33 (m, 2H), 7.26 (m, 1H), 7.10 (d, 1H, J=9.6 Hz), 5.11-4.98 (m, 1H), 4.83-4.62 (m, 1H), 4.48-4.36 (m, 2H), 3.96-3.60 (m, 2H), 3.45-3.37 (m, 2H), 2.81-2.76 (m, 2H), 2.41-2.28 (m, 2H), 2.05-2.03 (m, 3H), 1.85-1.81 (m, 3H), 1.74 (m, 3H), 1.40-0.94 (m, 11H), 0.57-0.51 (m, 1H)

### Preparation Example 4. Synthesis of Compound 4

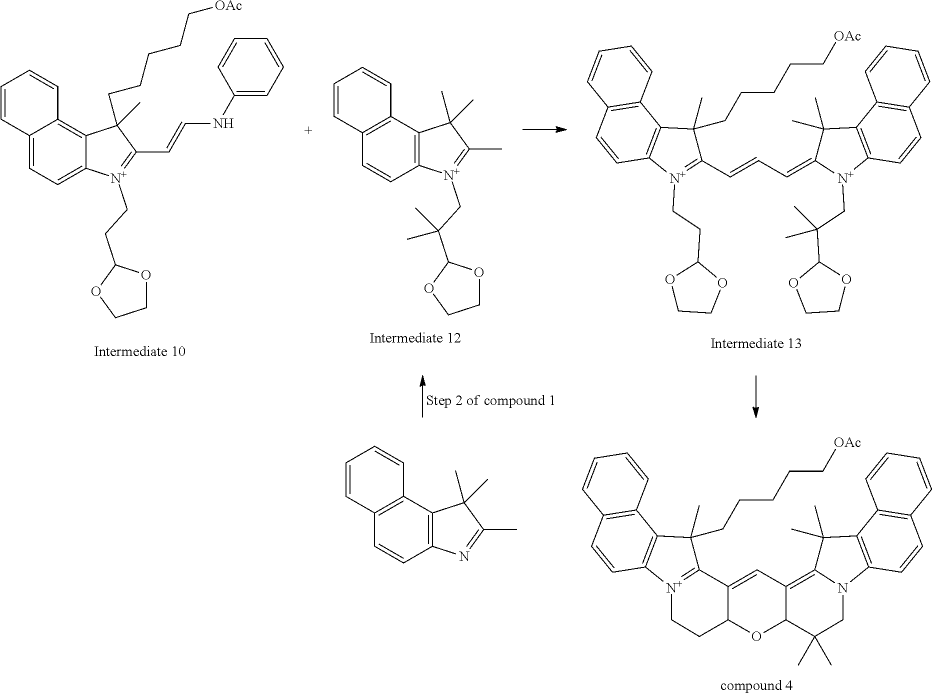 [SMILES: CC(=O)OCCCCCC1(C)C(/C=C/Nc2ccccc2)=[N+](CCC2OCCO2)c2ccc3ccccc3c21 ; CC1=[N+](CC(C)(C)C2OCCO2)c2ccc3ccccc3c2C1(C)C ; CC(=O)OCCCCCC1(C)C(/C=C/C=C2/[NH+](CC(C)(C)C3OCCO3)c3ccc4ccccc4c3C2(C)C)=[N+](CCC2OCCO2)c2ccc3ccccc3c21 ; CC1=Nc2ccc3ccccc3c2C1(C)C ; CC(=O)OCCCCCC1(C)C2=[N+](CCC3OC4C(=C5N(CC4(C)C)c4ccc6ccccc6c4C5(C)C)C=C23)c2ccc3ccccc3c21]

### Synthesis of Intermediate 13

Intermediate 10 of Compound 3 (6 g, 9.17 mmol), Intermediate 12 (synthesized using 1,1,2-trimethyl-1H-benzo[e]indole with reference to the preparation method of Intermediate 3 of Compound 1) (4.47 g, 9.17 mmol), acetic anhydride (9 mL), and pyridine (60 mL) were added to a 250 mL one-neck reactor, stirred at 50° C. for 24 hours, concentrated, and column purified (4.8 g).

### Synthesis of Compound 4

Intermediate 13 (4.8 g, 5.35 mmol), a 50% aqueous sulfuric acid solution (10 mL), and chloroform (50 mL) were added to a 250 mL one-neck reactor and stirred at room temperature for 24 hours. Water (100 mL) was added to the reactor and stirred vigorously to separate the organic layer. Afterward, anhydrous sodium sulfate was added, stirred for 5 minutes, and filtered, and the filtrate was concentrated, and column purified (0.9 g).

The ₁H-NMR data of the obtained Compound 4 is as follows.

1H-NMR (400 MHz, CDCl3) δ 8.44-8.40 (m, 1H), 8.15-8.12 (m, 2H), 8.00-7.97 (m, 4H), 7.65 (m, 2H), 7.54-7.39 (m, 4H), 5.15-5.02 (m, 1H), 4.93-4.71 (m, 1H), 4.46-4.37 (m, 2H), 4.16-3.79 (m, 2H), 3.44-3.42 (m, 2H), 2.91-2.75 (m, 2H), 2.42 (m, 2H), 2.19-2.06 (m, 9H), 1.46-1.29 (m, 5H), 1.27-1.118 (m, 2H), 0.99 (m, 4H), 0.64-0.55 (m, 1H)

### Preparation Example 5. Synthesis of Compound 5, Compound 18 and Compound 19

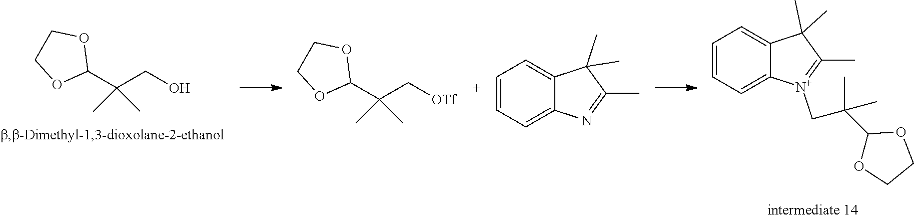

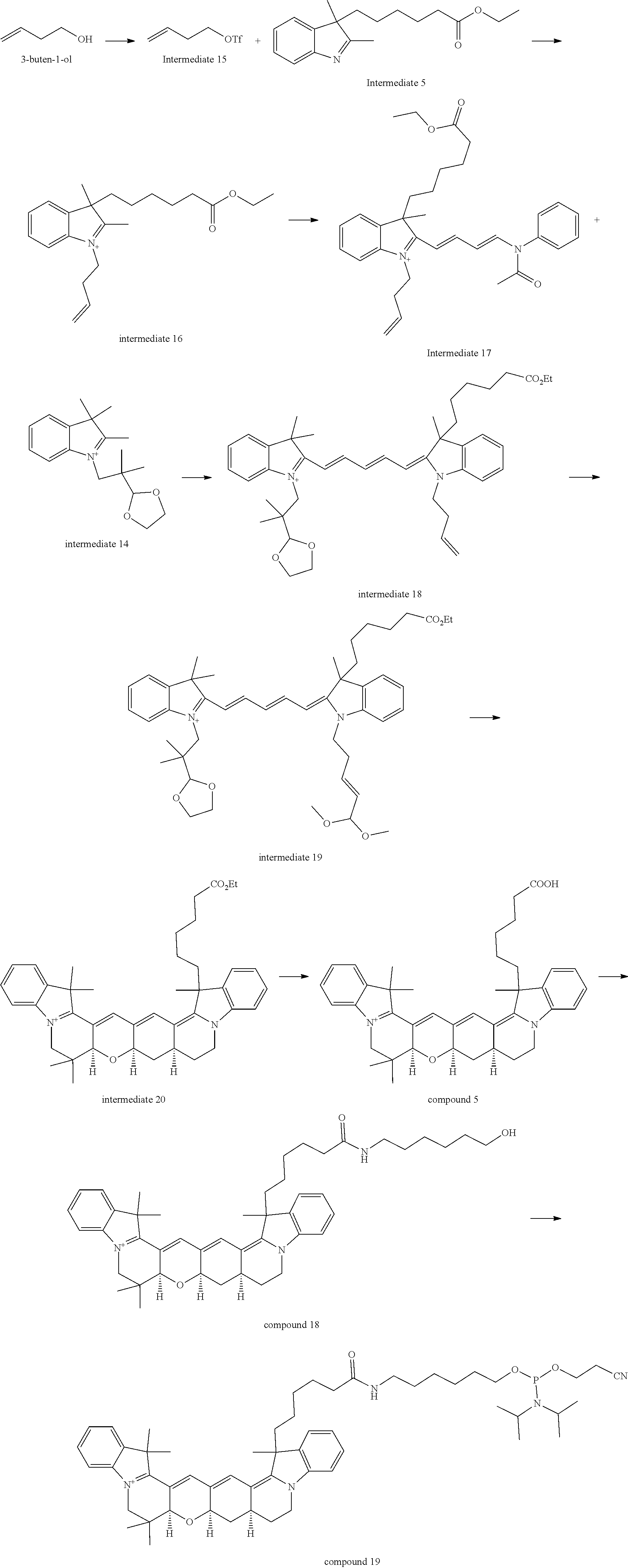

### Synthesis of Intermediate 16

Intermediate 15 (synthesized using 3-buten-1-ol with reference to Journal of the American Chemical Society (2004), 126(19), 6064-6071) (465 g, 2.27 mol), Intermediate 5 of Compound 2 (503.5 g, 1.75 mol), and 5 L of dichloromethane were added to a 10 L four-neck reactor and stirred at room temperature for 1 hour. The reaction solution was cooled, concentrated, and column purified (922 g, 107%).

### Synthesis of Intermediate 17

Intermediate 16 (922 g, 1.87 mol), malonaldehyde dianilide hydrochloride (630.9 g, 2.43 mol), and 6 L of acetic anhydride were added to a 10 L four-neck reactor and stirred under reflux for 1 hour. The reaction solution was cooled, concentrated, and column purified (1132 g, 91%).

### Synthesis of Intermediate 18

Intermediate 14 (synthesized with reference to the preparation method of Intermediates 2 and 3 in Compound 1) (341 g, 0.779 mol), Intermediate 17 (516.6 g, 0.779 mol), triethylamine (434.6 mL, 3.11 mol), and 5 L of dichloromethane were added to a 10 L four-neck reactor and stirred under reflux for 2 hours. The reaction solution was cooled, concentrated, and column purified (541 g, 85%).

### Synthesis of Intermediate 19

Intermediate 18 (541 g, 0.663 mol), acrolein dimethyl acetal (678 g, 6.63 mol), a second-generation Grubbs catalyst (253.6 g, 0.298 mmol), and 5 L of dichloromethane were added to a 10 L four-neck reactor and stirred under reflux for 24 hours. After cooling and concentrating the reaction solution, it was dissolved in methanol, passed through an Amberlite IRA-410 chloride foam resin, and extracted with dichloromethane and water. Anhydrous sodium sulfate was added to the organic layer, stirred for 5 minutes, and filtered, and the filtrate was concentrated, and column purified (330 g, 64%).

### Synthesis of Intermediate 20

Intermediate 19 (330 g, 0.436 mol) and 4 L of dichloromethane were added to a 10 L four-neck reactor. After cooling the reaction solution to −78° C., boron tribromide (414.5 mL, 4.36 mol) was slowly added. After stirring the reaction solution at the same temperature for 3 hours, 4 L of saturated aqueous sodium bicarbonate solution was slowly added, and the organic layer was extracted, concentrated, and dissolved in methanol again. After adding a 0.3 M aqueous hydrochloric acid solution and stirring at 60° C. for 2 hours, dichloromethane was added to extract the organic layer. Anhydrous sodium sulfate was added to the organic layer, stirred for 5 minutes, and filtered, and the filtrate was concentrated (271 g, 93%).

### Synthesis of Compound 5

Intermediate 20 (103.1 g, 0.154 mol) dissolved in 1 L of methanol was added to a 5 L four-neck reactor, and 800 ml of 1 M aqueous sodium hydroxide solution was added to the reactor and stirred at room temperature for 16 hours, and then a 2 M aqueous hydrochloric acid solution was added. After separating the organic layer by adding dichloromethane, anhydrous sodium sulfate was added to the organic layer, stirred for 5 minutes, and then filtered. Afterward, the filtrate was concentrated, and column purified (23.2 g, 23%).

### Synthesis of Compound 18

Compound 5 (23.2 g, 0.036 mol) and HATU (16.6 g, 0.043 mol) dissolved in 230 ml of dimethylformamide were added to a 1 L one-neck reactor, and N,N-diisopropylethylamine (12.6 mL, 0.072 mol) was added to the reactor. The mixture was stirred at room temperature for 10 minutes, and 6-aminohexanol (21.2 g, 0.181 mol) was added. The reaction solution was concentrated and extracted using dichloromethane and water. Anhydrous sodium sulfate was added to the organic layer, stirred for 5 minutes, and filtered, and the filtrate was concentrated and column-purified (22.8 g, 73%).

### Synthesis of Compound 19

Compound 18 (22.4 g 0.026 mol) dissolved in 200 ml of dichloromethane was added to a 1 L one-neck reactor, and then triethylamine (11 mL, 0.08 mol) was added. 2-cyanoethyl N,N′-diisopropylchlorophosphoramidite (8.8 g, 0.037 mol) was added to the reactor and stirred at room temperature for 30 minutes. After diluting the reaction solution with dichloromethane, a 10% aqueous sodium carbonate solution was added to separate the organic layer. Anhydrous sodium sulfate was added to the organic layer, stirred for 5 minutes, and filtered, and the filtrate was concentrated, and column purified (17.4 g, 63%).

The ₁H-NMR data of the obtained Compound 19 is as follows.

1H-NMR (400 MHz, CDCl3) δ 7.66 (s, 1H), 7.54 (s, 1H), 7.38-7.33 (m, 4H), 7.23-7.19 (m, 2H), 7.10 (d, 1H), 7.02-6.99 (m, 1H), 4.56-4.48 (m, 1H), 4.36-4.32 (m, 1H), 4.24-4.16 (m, 1H), 3.80-3.38 (m, 12H), 3.01 (s, 1H), 2.89 (s, 1H), 2.82-2.75 (m, 1H), 2.63 (t, 2H), 2.51-2.16 (m, 8H), 1.98-1.83 (m, 2H), 1.77-1.71 (m, 16H), 1.55-1.42 (m, 3H), 1.16-1.12 (m, 15H), 0.91 (s, 1H), 0.56 (s, 1H)

### Preparation Example 6. Synthesis of Compound 6, Compound 20 and Compound 21

### Synthesis of Intermediate 53

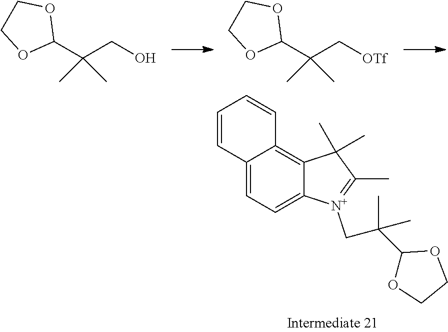

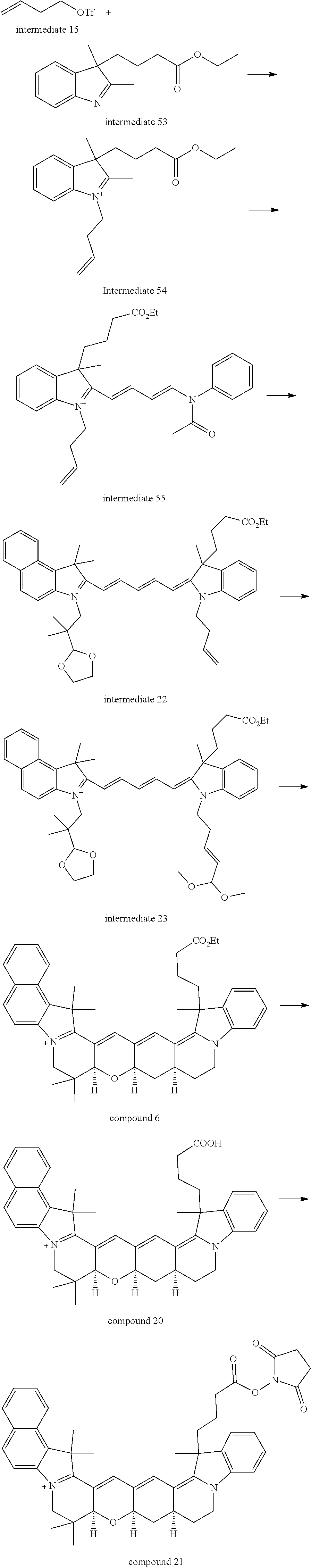

Intermediate 53 was synthesized in the same manner as the synthesis method of Intermediate 5 of Compound 2, except that ethyl 6-bromobutyrate (35 g, 0.179 mol) was used instead of ethyl 6-bromohexanoate.

### Synthesis of Intermediate 54

Intermediate 54 was synthesized in the same manner as the synthesis method of Intermediate 16 of Compound 5, except that Intermediate 53 (29 g, 0.112 mol) was used instead of Intermediate 5.

### Synthesis of Intermediate 55

Intermediate 55 was synthesized in the same manner as the synthesis method of Intermediate 17 of Compound 5, except that Intermediate 54 (20 g, 0.043 mol) was used instead of Intermediate 16.

### Synthesis of Intermediate 22

Intermediate 22 was synthesized in the same manner as the synthesis method of Intermediate 18 of Compound 5, except that Intermediate 21 (synthesized using 1,1,2-trimethyl-1H-benzo[e]indole instead of 2,3,3-trimethylindolenine in the preparation of Intermediate 3 of Compound 1) (9.3 g, 0.019 mol) was used instead of Intermediate 14, and Intermediate 55 (12 g, 0.019 mol) was used instead of Intermediate 17.

### Synthesis of Intermediate 23

Intermediate 23 was synthesized in the same manner as the synthesis method of Intermediate 19 of compound 5, except that Intermediate 22 (9.5 g, 0.011 mol) was used instead of Intermediate 18.

### Synthesis of Compound 6

Compound 6 was synthesized in the same manner as the synthesis method of Intermediate 20 of Compound 5, except that Intermediate 23 (7.6 g, 9.21 mmol) was used instead of Intermediate 19.

### Synthesis of Compound 20

Compound 20 was synthesized in the same manner as the synthesis method of Intermediate 20 of Compound 5, except that Compound 6 (2.6 g, 3.62 mmol) was used instead of Intermediate 19.

### Synthesis of Compound 21

Compound 6 (1.8 g, 2.61 mmol) and N,N,N′,N′-tetramethyl-O—(N-succinimidyl) uranium tetrafluoroborate (0.8 g, 2.61 mmol) dissolved in 50 ml of dimethylformamide were added to a 250 ml one-neck reactor, and triethylamine (0.8 mL, 7.83 mmol) was added to the reaction solution and stirred at room temperature for 2 hours. After concentrating the solvent, it was extracted with dichloromethane and washed with a 1 M aqueous hydrochloric acid solution. Anhydrous sodium sulfate was added to the organic layer, stirred for 5 minutes, and filtered, and the filtrate was concentrated, and column purified (1.6 g, 79%).

The ₁H-NMR data of the obtained compound 21 is as follows.

1H-NMR (400 MHz, CDCl3) δ 8.19 (d, 1H), 7.98 (d, 1H), 7.92 (d, 2H), 7.70 (d, 1H), 7.61 (t, 1H), 7.46 (t, 1H), 7.37-7.31 (m, 3H), 7.24 (d, 1H), 7.09-7.06 (m, 1H), 4.54-4.51 (m, 1H), 4.4 (d, 1H), 4.22-4.16 (m, 1H), 3.84-3.74 (m, 2H), 2.80-2.78 (m, 5H), 2.52-2.38 (m, 4H), 2.17 (s, 1H), 2.08-2.03 (m, 4H), 1.78-1.74 (t, 3H), 1.62 (s, 6H), 1.60-1.48 (m, 3H), 1.38 (s, 3H), 1.33-1.25 (m, 1H), 0.91-0.90 (m, 1H), 0.64-0.60 (m, 1H)

### Preparation Example 7. Synthesis of Compound 7

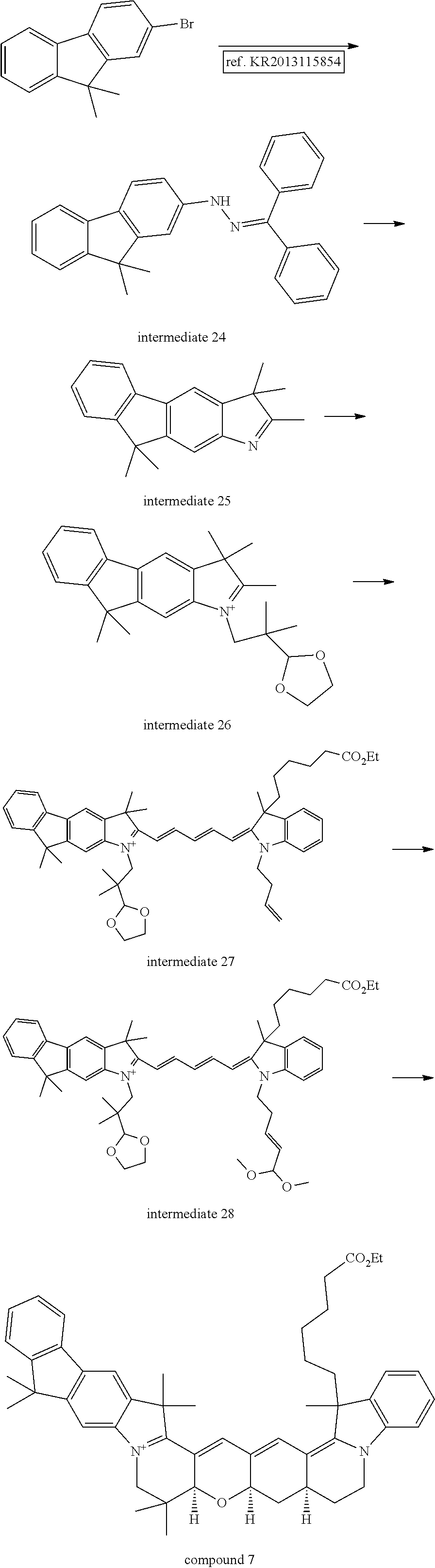

### Synthesis of Intermediate 25

Intermediate 24 (32.4 g, 0.083 mol) and 3-methyl-2-butanone (22.5 g, 0.25 mol) dissolved in 300 ml of ethanol were added to a 2 L one-neck reactor and stirred at room temperature. 80 ml of hydrochloric acid was added to the reaction solution and stirred under reflux for 24 hours, and after cooling and concentrating the reaction solution, acetonitrile was added, dissolved, and concentrated. After adding 300 ml of ethyl acetate, the resulting solid was filtered, and 500 ml of ethyl acetate and 500 ml of saturated aqueous sodium bicarbonate solution were added to the filtered solid and stirred. After obtaining the organic layer, anhydrous magnesium sulfate was added, stirred for 5 minutes, filtered, and concentrated (11.4 g, 50%).

### Synthesis of Intermediate 26

Intermediate 26 was synthesized in the same manner as the synthesis method of Intermediate 3 of Compound 1, except that Intermediate 25 (12.1 g, 0.054 mol) was used instead of 2,3,3-trimethylindolenine.

### Synthesis of Intermediate 27

Intermediate 27 was synthesized in the same manner as the synthesis method of Intermediate 18 of Compound 5, except that Intermediate 26 (6.1 g, 0.011 mol) was used instead of Intermediate 14.

### Synthesis of Intermediate 28

Intermediate 28 was synthesized in the same manner as the synthesis method of Intermediate 19 of Compound 5, except that Intermediate 27 (7.8 g, 8.38 mmol) was used instead of Intermediate 18.

### Synthesis of Compound 7

Compound 7 was synthesized in the same manner as the synthesis method of Intermediate 20 of Compound 5, except that instead of Intermediate 19, Intermediate 28 (5.8 g, 6.50 mmol) was used (2 g, 39%).

The ₁H-NMR data of the obtained Compound 7 is as follows.

1H-NMR (400 MHz, CDCl3) δ 7.72 (d, 1H), 7.67-7.65 (m, 1H), 7.43 (d, 1H), 7.39-7.29 (m, 4H), 7.26 (d, 1H), 7.15-7.11 (m 1H), 7.09 (d, 1H), 4.60-4.40 (m, 3H), 4.24-3.92 (m, 5H), 3.80-3.76 (m, 1H), 3.60-3.58 (m, 1H), 2.96-2.75 (m, 1H), 2.68-2.39 (m, 1H), 2.31-2.22 (m, 1H), 2.16-2.13 (m, 3H), 2.04 (s, 1H), 1.93-1.91 (m, 2H), 1.88-1.86 (m, 3H), 1.82-1.80 (m, 2H), 1.75-1.67 (m, 1H), 1.60 (s, 5H), 1.52-1.50 (m, 8H), 1.46-1.42 (m, 2H), 1.36 (s, 2H), 1.27 (t, 2H), 1.22-1.17 (m, 3H), 0.92-0.88 (m, 1H), 0.59-0.45 (m, 1H)

### Preparation Example 8. Synthesis of Compound 8

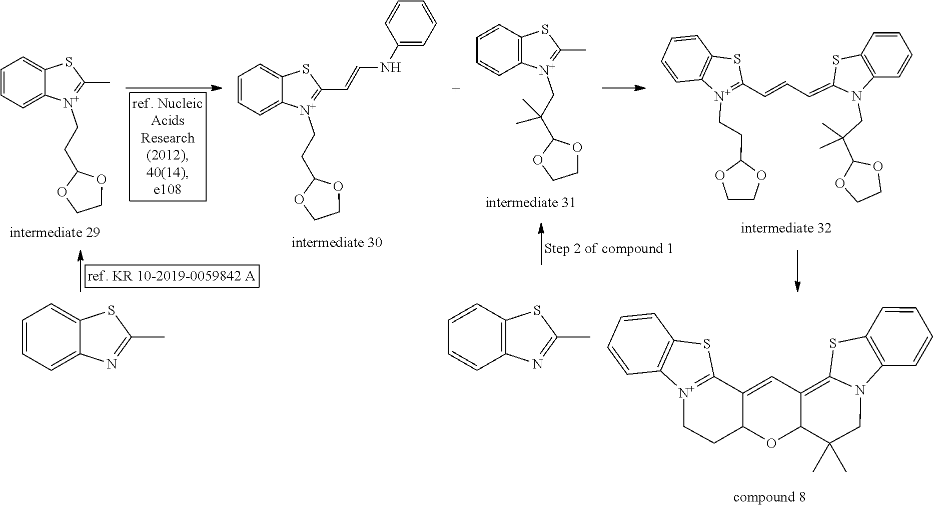

### Synthesis of Intermediate 29

Intermediate 29 was synthesized using 2-methylbenzothiazole (synthesized with reference to Korean Unexamined Patent Publication No. 10-2019-0059842).

### Synthesis of Intermediate 32

Intermediate 30 (synthesized with reference to Nucleic Acid Research (2012), 40(14), e108) (2.25 g, 5 mmol), Intermediate 31 (synthesized with reference to the synthesis of Intermediate 3 of Compound 1 using 2-methylbenzothiazole) (2 g, 5 mmol), acetic anhydride (5 mL), and pyridine (20 mL) were added to a 250 mL one-neck reactor, stirred at 50° C. for 24 hours, cooled, and concentrated.

### Synthesis of Compound 8

Intermediate 32 (11.8 g, 0.02 mol), a 50% aqueous sulfuric acid solution (24 mL), and chloroform (120 mL) were added to a 250 mL one-neck reactor and stirred at room temperature for 24 hours. Water (100 mL) was added to the reactor and stirred vigorously to separate the organic layer. Anhydrous sodium sulfate was added, stirred for 5 minutes, and filtered, and the filtrate was concentrated, and column purified (45 mg).

The ₁H-NMR data of the obtained Compound 8 is as follows.

¹H-NMR (400 MHz, CDCl₃) δ 8.01-7.99 (m, 2H), 7.68-7.63 (m, 3H), 7.556-7.55 (m, 2H), 7.42-7.40 (m, 2H), 4.76-4.69 (m, 1H), 4.65-4.58 (m, 1H), 4.51 (s, 1H), 4.35 (d, 1H, J=13.2 Hz), 4.13-4.07 (m, 1H), 3.87 (d, 1H, J=13.2 Hz), 2.61-2.52 (m, 1H), 2.09-2.00 (m, 1H), 1.31 (s, 3H), 0.89 (s, 3H)

### Preparation Example 9. Synthesis of Compound 9

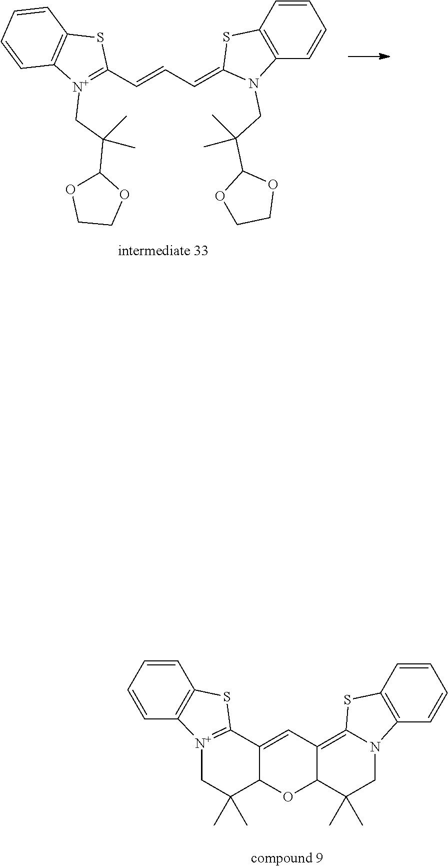 [SMILES: CC(C)(CN1/C(=C/C=C/c2sc3ccccc3[n+]2CC(C)(C)C2OCCO2)Sc2ccccc21)C1OCCO1 ; CC1(C)C[n+]2c(sc3ccccc32)C2=CC3=C4Sc5ccccc5N4CC(C)(C)C3OC21]

Intermediate 33 (refer to the synthesis of Intermediate 31 of Compound 8) (6.7 g, 9 mmol), a 50% aqueous sulfuric acid solution (14 mL), and chloroform (70 mL) were added to a 100 mL one-neck reactor and stirred at room temperature for 24 hours. Water (100 mL) was added to the reactor and stirred vigorously to separate the organic layer. Anhydrous sodium sulfate was added, stirred for 5 minutes, and filtered, and the filtrate was concentrated, and column purified (13 mg).

The ₁H-NMR data of the obtained Compound 9 is as follows.

¹H-NMR (400 MHz, CDCl₃) δ 7.69 (d, 2H, J=8.0 Hz), 7.54-7.49 (m, 3H), 7.40-7.38 (m, 4H), 4.55 (s, 2H), 4.00-3.91 (m, 4H), 1.42 (s, 6H), 0.98 (s, 6H)

### Preparation Example 10. Synthesis of Compound 10

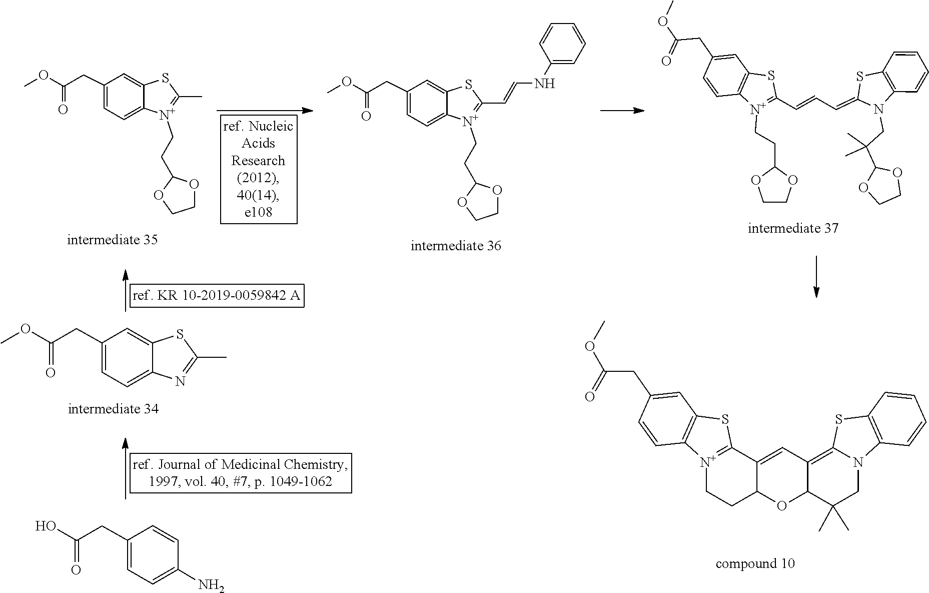

### Synthesis of Intermediate 35

Intermediate 35 was synthesized using Intermediate 34 (synthesized with reference to Korean Unexamined Patent Publication No. 10-2019-0059842).

### Synthesis of Intermediate 37

Intermediate 36 (synthesized with reference to Nucleic Acid Research (2012), 40(14), e108) (5.17 g, 5 mmol), Intermediate 31 (4 g, 5 mmol), acetic anhydride (5 mL), and pyridine (50 mL) were added to a 250 mL one-neck reactor, stirred at 50° C. for 24 hours, cooled, and concentrated.

### Synthesis of Compound 10

Intermediate 37 (7 g, 0.01 mol), a 50% aqueous sulfuric acid solution (15 mL), and chloroform (200 mL) were added to a 250 mL one-neck reactor and stirred at room temperature for 24 hours. Water (100 mL) was added to the reactor and stirred vigorously to separate the organic layer. Anhydrous sodium sulfate was added, stirred for 5 minutes, and filtered, and the filtrate was concentrated and column purified (50 mg).

The ₁H-NMR data of the obtained Compound 10 is as follows.

¹H-NMR (400 MHz, CDCl₃) δ 7.6-7.38 (m, 3H), 4.87-4.6.1 (m, 1H), 4.54 (s, 1H), 4.89-4.82 (m, 1H), 4.19-4.15 (m, 3H), 3.97-3.82 (m, 2H), 3.73 (s, 3H), 2.76-2.70 (m, 1H), 2.13-2.20 (m, 1H), 1.40 (s, 3H), 0.96 (s, 3H)

### Preparation Example 11. Synthesis of Compound 11

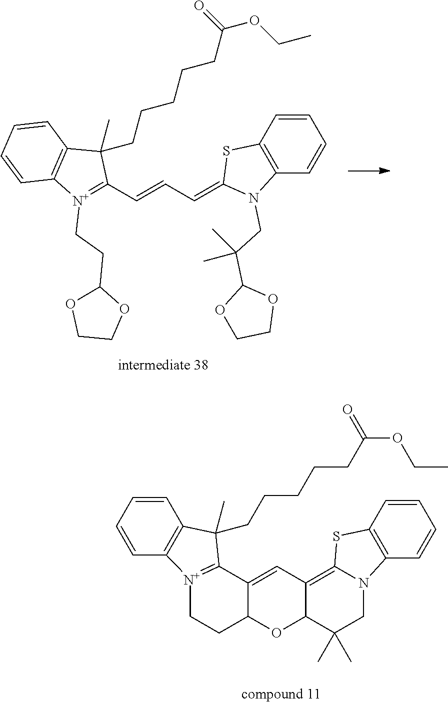 [SMILES: CCOC(=O)CCCCCC1(C)C(/C=C/C=C2\Sc3ccccc3N2CC(C)(C)C2OCCO2)=[N+](CCC2OCCO2)c2ccccc21 ; CCOC(=O)CCCCCC1(C)C2=[N+](CCC3OC4C(=C5Sc6ccccc6N5CC4(C)C)C=C23)c2ccccc21]

Intermediate 38 (refer to the synthesis of Intermediate 7 of Compound 2) (9 g, 11 mmol), a 50% aqueous sulfuric acid solution (10 mL), and chloroform (100 mL) were added to a 100 mL one-neck reactor and stirred at room temperature for 24 hours. Water (100 mL) was added to the reactor and stirred vigorously to separate the organic layer. Anhydrous sodium sulfate was added, stirred for 5 minutes, and filtered, and the filtrate was concentrated, and column purified (130 mg).

The ₁H-NMR data of the obtained Compound 11 is as follows.

¹H-NMR (400 MHz, CDCl₃) δ 7.79-7.74 (m, 2H), 7.62-7.57 (m, 2H), 7.46-7.42 (m, 2H), 7.30-7.19 (m, 2H), 7.04 (m, 1H, J=7.6 Hz), 4.73-4.69 (m, 1H), 4.63 (s, 1H), 4.17-4.12 (m, 3H), 4.02 (q, 2H, J=6.8 Hz), 3.90-3.82 (m, 1H), 2.67-2.63 (m, 1H), 2.14-1.98 (m, 3H), 1.67 (s, 3H), 1.48-1.42 (m, 5H), 1.25-1.14 (m, 5H), 0.98 (s, 1H), 0.95-0.88 (m, 1H), 0.70-0.59 (m, 1H)

### Preparation Example 12. Synthesis of Compound 12

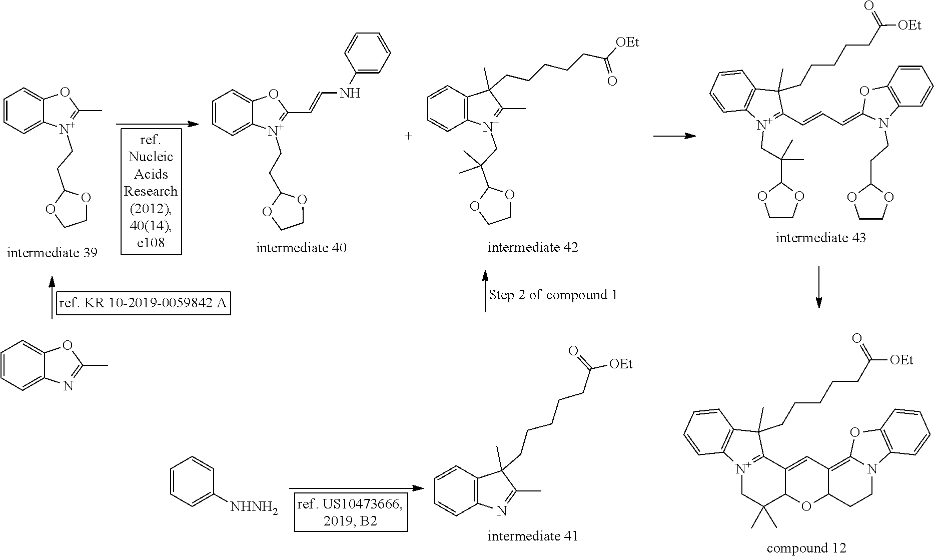

### Synthesis of Intermediate 39

Intermediate 39 was synthesized using 2-methylbenzoxazole (synthesized with reference to Korean Unexamined Patent Publication No. 10-2019-0059842).

### Synthesis of Intermediate 43

Intermediate 40 (synthesized with reference to Nucleic Acid Research (2012), 40(14), e108) (3.28 g, 7 mmol), Intermediate 42 (synthesized using Intermediate 41 with reference to the synthesis of Intermediate 3) (4.0 g, 7 mmol), acetic anhydride (9 mL), and pyridine (30 mL) were added to a 250 mL one-neck reactor, stirred at 50° C. for 24 hours, cooled, and concentrated.

### Synthesis of Compound 12

Intermediate 43 (5.6 g, 0.02 mol), a 50% aqueous sulfuric acid solution (10 mL), and chloroform (10 mL) were added to a 250 mL one-neck reactor and stirred at room temperature for 24 hours. Water (100 mL) was added to the reactor and stirred vigorously to separate the organic layer. Anhydrous sodium sulfate was added, stirred for 5 minutes, and filtered, and the filtrate was concentrated, and column purified (400 mg).

The ₁H-NMR data of the obtained Compound 12 is as follows.

¹H-NMR (400 MHz, CDCl₃) δ 8.05 (d, 1H, J=10.0 Hz), 7.62-7.57 (m, 2H), 7.48-7.20 (m, 5H), 6.99 (d, 1H, J=8.0 Hz), 4.93-4.89 (m, 1H), 4.565 (m, 1H), 4.43-4.40 (m, 2H), 4.05-3.98 (m, 2H), 3.66-3.55 (m, 2H), 2.76 (m, 1H), 2.20-2.12 (m, 3H), 1.68 (s, 3H), 1.48-1.42 (m, 2H), 1.35 (s, 3H), 1.27-1.15 (m, 5H), 0.95-0.90 (m, 3H), 0.89-0.61 (m, 2H)

### Preparation Example 13. Synthesis of Compound 13

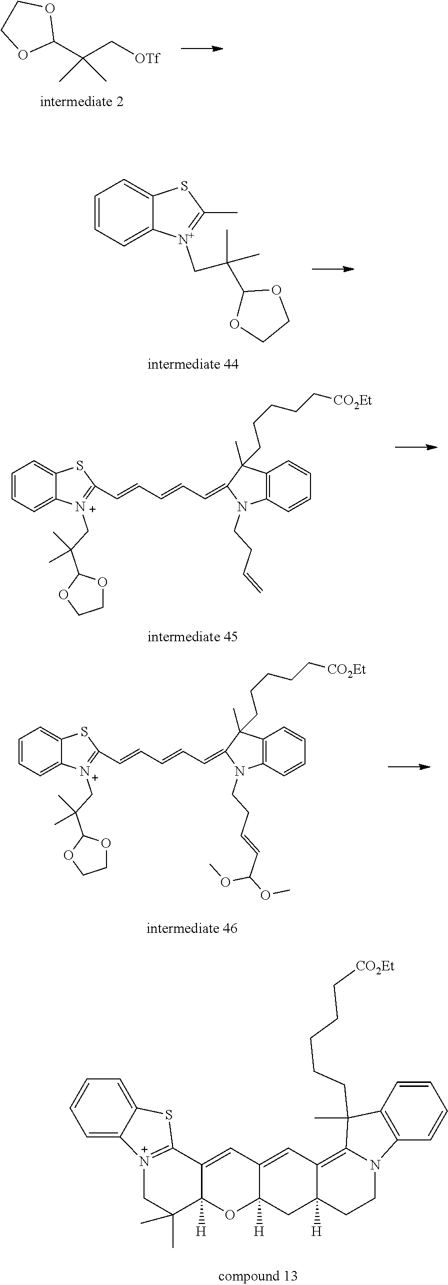 [SMILES: CC(C)(COS(=O)(=O)C(F)(F)F)C1OCCO1 ; Cc1sc2ccccc2[n+]1CC(C)(C)C1OCCO1 ; C=CCCN1/C(=C/C=C/C=C/c2sc3ccccc3[n+]2CC(C)(C)C2OCCO2)C(C)(CCCCCC(=O)OCC)c2ccccc21 ; CCOC(=O)CCCCCC1(C)\C(=C/C=C/C=C/c2sc3ccccc3[n+]2CC(C)(C)C2OCCO2)N(CC/C=C/C(OC)OC)c2ccccc21 ; CCOC(=O)CCCCCC1(C)C2=C3C=C4C=C5c6sc7ccccc7[n+]6CC(C)(C)[C@@H]5O[C@@H]4C[C@@H]3CCN2c2ccccc21]

### Synthesis of Intermediate 44

Intermediate 44 was synthesized in the same manner as the synthesis method of Intermediate 3, except that 2-methylbenzothiazole (25 g, 0.167 mol) was used instead of 2,3,3-trimethylindolenine.

### Synthesis of Intermediate 45

Intermediate 45 was synthesized in the same manner as the synthesis method of Intermediate 18, except that Intermediate 44 (14.3 g, 0.035 mol) was used instead of Intermediate 14.

### Synthesis of Intermediate 46

Intermediate 46 was synthesized in the same manner as the synthesis method of Intermediate 19, except that Intermediate 45 (4.3 g, 5.34 mmol) was used instead of Intermediate 18.

### Synthesis of Compound 13

Compound 13 was synthesized in the same manner as the synthesis method of Intermediate 20, except that instead of Intermediate 19, Intermediate 46 (1.7 g, 2.16 mmol) was used (0.16 g, 11%).

The ₁H-NMR data of the obtained Compound 13 is as follows.

¹H-NMR (400 MHz, CDCl₃) δ 8.12-8.07 (dd, 1H), 7.77-7.75 (m, 1H), 7.53 (d, 2H), 7.47 (s, 1H), 7.43-7.39 (m, 2H), 7.34-7.30 (m, 1H), 7.16-7.13 (m, 1H), 6.97-6.93 (m, 1H), 4.67-4.52 (m, 2H), 4.16-4.02 (m, 4H), 3.74-3.70 (m, 1H), 3.60 (d, 1H), 2.18-2.12 (m, 2H), 1.71 (d, 2H), 1.65 (s, 8H), 1.22-1.97 (m, 4H), 0.94 (b, 1H), 0.60 (b, 1H)

### Preparation Example 14. Synthesis of Compound 50

### Synthesis of Intermediate 47

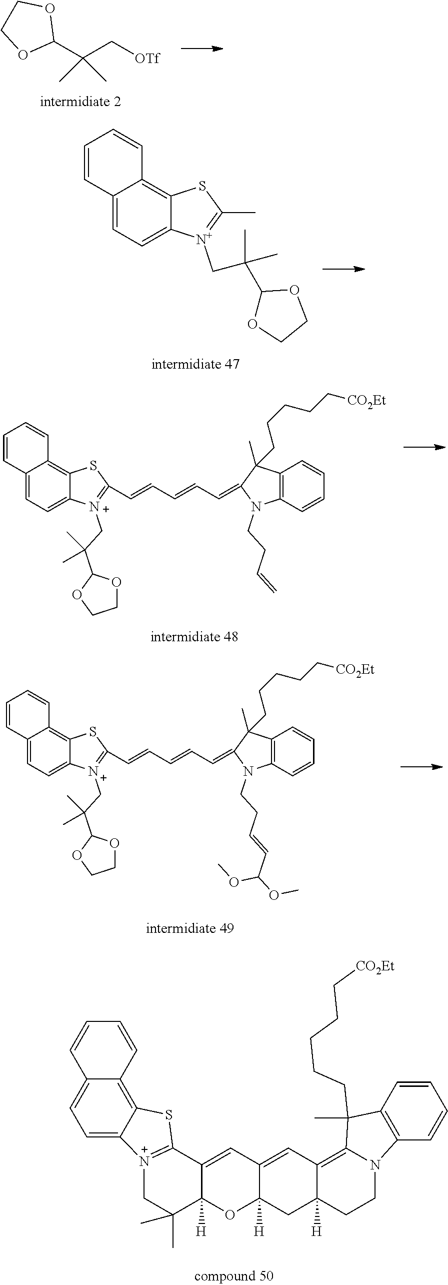 [SMILES: CC(C)(COS(=O)(=O)C(F)(F)F)C1OCCO1 ; Cc1sc2c3ccccc3ccc2[n+]1CC(C)(C)C1OCCO1 ; C=CCCN1/C(=C/C=C/C=C/c2sc3c4ccccc4ccc3[n+]2CC(C)(C)C2OCCO2)C(C)(CCCCCC(=O)OCC)c2ccccc21 ; CCOC(=O)CCCCCC1(C)\C(=C/C=C/C=C/c2sc3c4ccccc4ccc3[n+]2CC(C)(C)C2OCCO2)N(CC/C=C/C(OC)OC)c2ccccc21 ; CCOC(=O)CCCCCC1(C)C2=C3C=C4C=C5c6sc7c8ccccc8ccc7[n+]6CC(C)(C)[C@@H]5O[C@@H]4C[C@@H]3CCN2c2ccccc21]

Intermediate 47 was synthesized in the same manner as the synthesis method of Intermediate 3, except that 2-methylnaphtho[2,1-d] thiazole (33.3 g, 0.167 mol) was used instead of 2,3,3-trimethylindolenine.

### Synthesis of Intermediate 48

Intermediate 48 was synthesized in the same manner as the synthesis method of Intermediate 18, except that Intermediate 47 (16 g, 0.035 mol) was used instead of Intermediate 14.

### Synthesis of Intermediate 49

Intermediate 49 was synthesized in the same manner as the synthesis method of Intermediate 19, except that Intermediate 48 (4.6 g, 5.34 mmol) was used instead of Intermediate 18.

### Synthesis of Compound 50

Compound 50 was synthesized in the same manner as the synthesis method of Intermediate 20, except that instead of Intermediate 19, Intermediate 49 (1.8 g, 2.16 mmol) was used (0.21 g, 13%).

The ₁H-NMR data of the obtained Compound 50 is as follows.

¹H-NMR (400 MHz, CDCl₃) δ 8.41 (d, 1H), 8.20 (d, 1H), 8.09-8.01 (dd, 1H), 7.80-7.78 (m, 1H), 7.59 (d, 2H), 7.51 (s, 1H), 7.48-7.42 (m, 2H), 7.36-7.33 (m, 1H), 7.18-7.15 (m, 1H), 6.99-6.95 (m, 1H), 4.70-4.55 (m, 2H), 4.19-4.05 (m, 4H), 3.78-3.73 (m, 1H), 3.63 (d, 1H), 2.19-2.13 (m, 2H), 1.75 (d, 2H), 1.68 (s, 8H), 1.24-1.19 (m, 4H), 0.96 (b, 1H), 0.70 (b, 1H)

### Preparation Example 15. Synthesis of Compound 51

### Synthesis of Intermediate 50

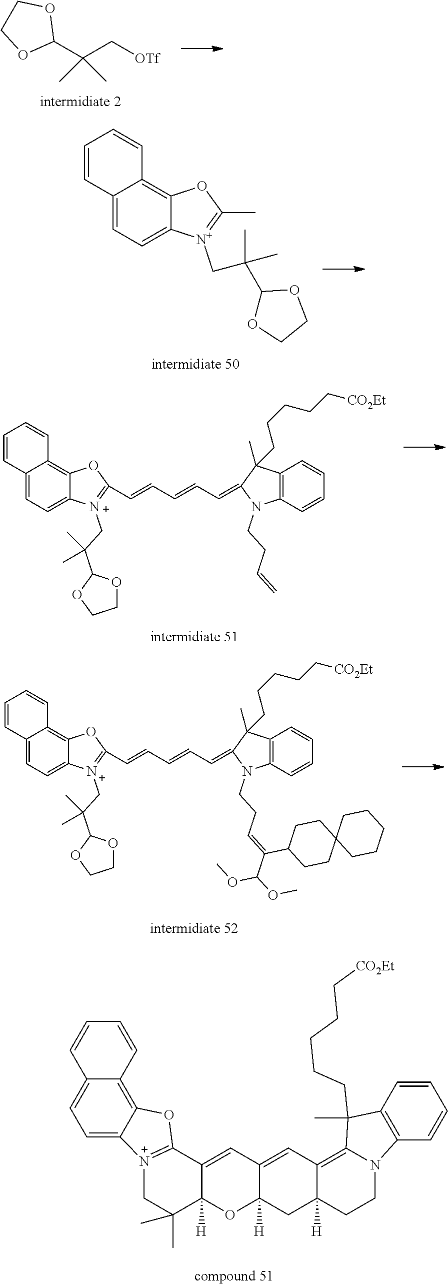 [SMILES: CC(C)(COS(=O)(=O)C(F)(F)F)C1OCCO1 ; Cc1oc2c3ccccc3ccc2[n+]1CC(C)(C)C1OCCO1 ; C=CCCN1/C(=C/C=C/C=C/c2oc3c4ccccc4ccc3[n+]2CC(C)(C)C2OCCO2)C(C)(CCCCCC(=O)OCC)c2ccccc21 ; CCOC(=O)CCCCCC1(C)\C(=C/C=C/C=C/c2oc3c4ccccc4ccc3[n+]2CC(C)(C)C2OCCO2)N(CC/C=C(\C2CCC3(CCCCC3)CC2)C(OC)OC)c2ccccc21 ; CCOC(=O)CCCCCC1(C)C2=C3C=C4C=C5c6oc7c8ccccc8ccc7[n+]6CC(C)(C)[C@@H]5O[C@@H]4C[C@@H]3CCN2c2ccccc21]

Intermediate 50 was synthesized in the same manner as the synthesis method of Intermediate 3, except that 2-methylnaphtho[2,1-d]oxazole (30.6 g, 0.167 mol) was used instead of 2,3,3-trimethylindolenine.

### Synthesis of Intermediate 51

Intermediate 51 was synthesized in the same manner as the synthesis method of Intermediate 18, except that Intermediate 50 (15.5 g, 0.035 mol) was used instead of Intermediate 14.

### Synthesis of Intermediate 52

Intermediate 52 was synthesized in the same manner as the synthesis method of Intermediate 19, except that Intermediate 51 (4.5 g, 5.36 mmol) was used instead of Intermediate 18.

### Synthesis of Compound 51

Compound 51 was synthesized in the same manner as the synthesis method of Intermediate 20, except that instead of Intermediate 19, Intermediate 52 (1.7 g, 2.13 mmol) was used (0.16 g, 11%).

The 1H-NMR data of the obtained Compound 51 is as follows.

¹H-NMR (400 MHz, CDCl₃) δ 8.30 (d, 1H), 8.10 (d, 1H), 8.02-7.99 (dd, 1H), 7.71-7.68 (m, 1H), 7.48 (d, 2H), 7.41 (s, 1H), 7.36-7.31 (m, 2H), 7.27-7.30 (m, 1H), 7.12-7.09 (m, 1H), 6.89-6.85 (m, 1H), 4.60-4.64 (m, 2H), 4.05-4.01 (m, 4H), 3.67-3.62 (m, 1H), 3.52 (d, 1H), 2.09-2.03 (m, 2H), 1.66 (d, 2H), 1.58 (s, 8H), 1.14-1.08 (m, 4H), 0.92 (b, 1H), 0.68 (b, 1H)

The photophysical properties measured under DMSO solvent for Compounds 1 to 13, Compound 50, and Compound 51, synthesized according to the Preparation Examples described above, are shown in Table 1 below.

TABLE 1

| | | | Absorption | Quantum yield |
|---|---|---|---|---|
| Classification | UV (nm) | PL (nm) | coefficient (ε) | (Φ) |
| Compound 1 | 566 | 582 | 116,000 | 0.96 |
| Compound 2 | 566 | 581 | 106,000 | — |
| Compound 3 | 586 | 602 | 109,000 | 0.81 |
| Compound 4 | 604 | 620 | 109,000 | 0.79 |
| Compound 5 | 674 | 691 | 148,000 | — |
| Compound 6 | 692 | 711 | 141,000 | 0.54 |
| Compound 7 | 696 | 720 | 145,000 | 0.41 |
| Compound 8 | 582 | 595 | 156,000 | 0.84 |
| Compound 9 | 582 | 596 | 117,000 | 0.87 |
| Compound 10 | 584 | 596 | 70,000 | 0.84 |
| Compound 11 | 570 | 587 | 87,000 | 0.85 |
| Compound 12 | 530 | 550 | 94,000 | 0.88 |
| Compound 13 | 682 | 703 | 112,000 | — |
| Compound 50 | 700 | 723 | 108,000 | — |
| Compound 51 | 660 | 686 | 110,000 | — |

### Preparation Example 16. Synthesis of Dual-Labeled Probe (Oligonucleotide)

Using the Universal UnyLinker Support (ChemGenes, 500A), each of 5′-GCG GGA GAT GAT ATG GAC TT-3′ as a forward primer and 5′-CCG TCT GAG ATG CAT GAA TAC-3′ as a reverse primer for the black queen cell virus (BQCV) was synthesized at 1 μmol scale.

First, 5′-CCA TCT TTA TCG GTA CGC CGC CC-quencher-3′ was synthesized as a single labeled probe using quencher-CPG (synthesized with reference to Korean Unexamined Patent Publication No. 10-2020-0067733), and then using the MerMade™ 48×DNA synthesizer, Compound 2, Compound 5, Compound 11, Compound 12, Compound 13, Compound 51, Cy3™, and Cy5™ were labeled as reporters at the 5′ end of 5′-CCA TCT TTA TCG GTA CGC CGC CC-quencher-3′, respectively, and a quencher was labeled at the 3′ end to synthesize a dual-labeled probe.

For comparative experiments with Compound 6, Compound 7, and Compound 50, in Comparative Example 3, a dual-labeled probe containing the same probe sequence was synthesized by labeling Cy5.5™ at the 5′ end and Black Hole Quencher® 3 (BHQ3, LGC Biosearch Technologies), a commercially available quencher including an emission spectrum region, at the 3′ end.

The forms of the synthesized dual-labeled probes are shown in Table 2 below.

TABLE 2

| Classification | Dual-labeled probe |
|---|---|
| Example 1 | 5′-Compound 2-SEQ 3-Quencher-3′ |
| Example 2 | 5′-Compound 5-SEQ 3-Quencher-3′ |
| Example 3 | 5′-Compound 6-SEQ 3-BHQ3-3′ |
| Example 4 | 5′-Compound 7-SEQ 3-BHQ3-3′ |
| Example 5 | 5′-Compound 11-SEQ 3-Quencher-3′ |
| Example 6 | 5′-Compound 12-SEQ 3-Quencher-3′ |
| Example 7 | 5′-Compound 13-SEQ 3-Quencher-3′ |
| Example 8 | 5′-Compound 50-SEQ 3-BHQ3-3′ |
| Example 9 | 5′-Compound 51-SEQ 3-Quencher-3′ |
| Comparative | 5′-Cy3-SEQ 3-Quencher-3′ |
| Example 1 | |
| Comparative | 5′-Cy5-SEQ 3-Quencher-3′ |
| Example 2 | |
| Comparative | 5′-Cy5.5-SEQ 3-BHQ3-3′ |
| Example 3 | |

*SEQ 1: GCG GGA GAT GAT ATG GAC TT (a sequence for a forward primer using Preparation Example 16)
*SEQ 2: CCG TCT GAG ATG CAT GAA TAC (a sequence for a reverse primer using Preparation Example 16)
*SEQ 3: CCA TCT TTA TCG GTA CGC CGC CC (a sequence for a probe using Preparation Example 16)

The structure of the quencher-CPG used in Preparation Example 16 is as follows.

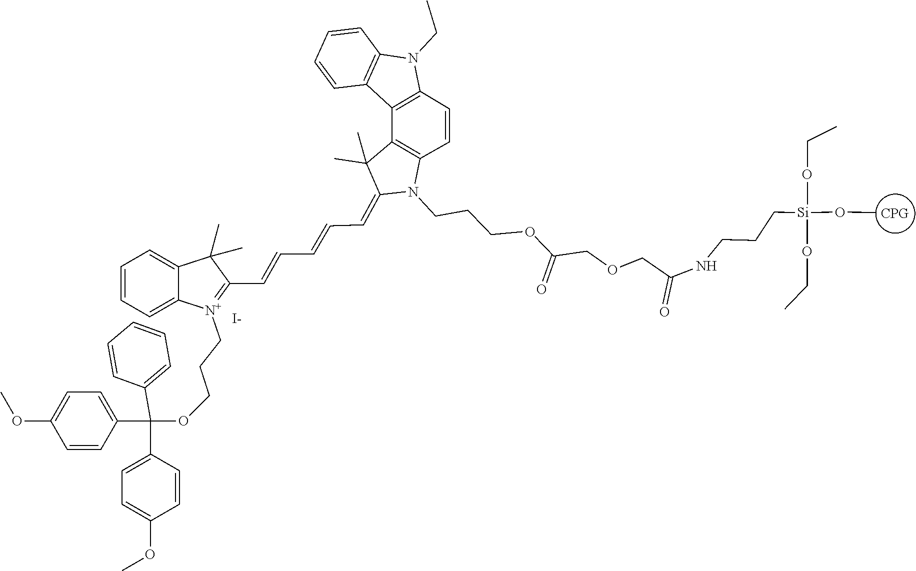

The absorption/emission wavelength ranges of Compound 2, Compound 5, Compound 6, Compound 7, Compound 11, Compound 12, Compound 13, Compound 50, Compound 51, Cy3™, Cy5™, and Cy5.5™ used as reporters in Preparation Example 16 are shown in Table 3 below, and the absorption wavelength ranges of the quencher and BHQ3 are shown in Table 4 below.

TABLE 3

| Reporter | Excitationₘₐₓ (nm) | Emissionₘₐₓ (nm) |
|---|---|---|
| Compound 2 | 566 | 581 |
| Compound 5 | 674 | 691 |
| Compound 6 | 692 | 711 |
| Compound 7 | 696 | 720 |
| Compound 11 | 570 | 587 |
| Compound 12 | 530 | 550 |
| Compound 13 | 682 | 703 |
| Compound 50 | 700 | 723 |
| Compound 51 | 660 | 686 |
| Cy3 | 550 | 565 |
| Cy5 | 648 | 670 |
| Cy5.5 | 689 | 713 |

TABLE 4

| Classification | λ_{Max} (nm) | ε (mol⁻¹ · cm⁻¹) |
|---|---|---|
| Quencher | 580-710 | 140,000 |
| BHQ3 | 620-730 | 42,000 |

### Experimental Example. Real-Time PCR Experiment Using Dual-Labeled

### Probe

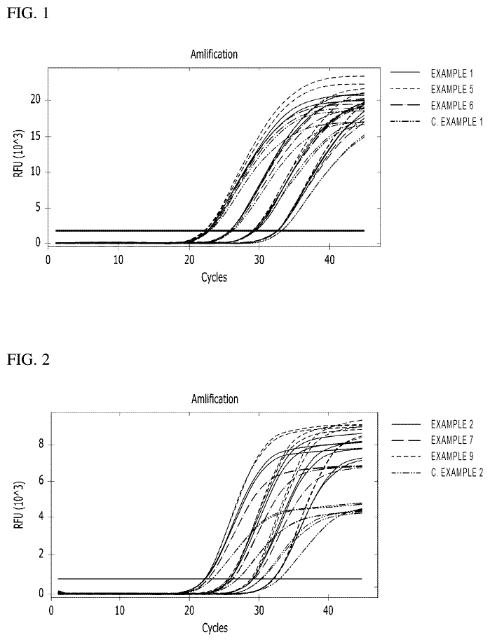

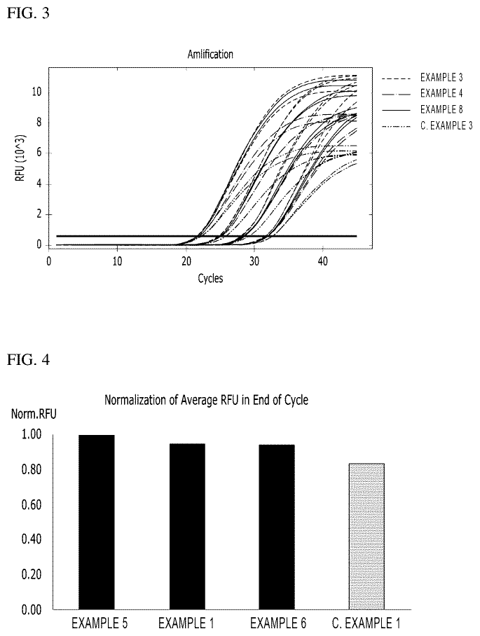

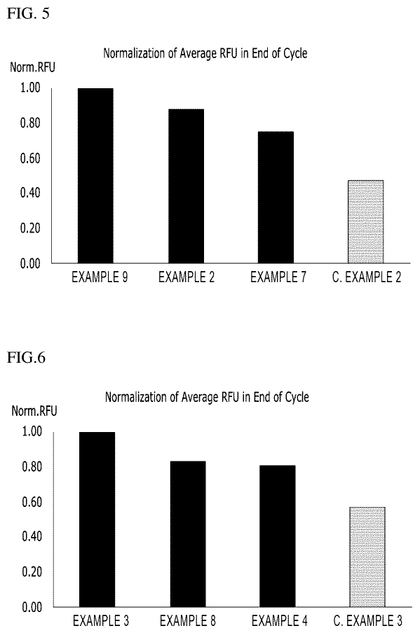

Real-time PCR was repeated twice on black queen cell virus (BQCV) plasmid DNA using each dual-labeled probe synthesized according to Preparation Example 16 with the composition shown in Table 5 below (using CFX-96 from Bio-Rad). The real-time PCR results are shown in FIGS. 1 to 6.

TABLE 5

| Classification | Content (μl) |
|---|---|
| (Bioline)SensiFAST ™ Probe No-ROX Mix (2X) | 10 |
| BQCV plasmid DNA (5000 copies/μl) | 1 |
| BQCV F/R primer mix (10 pmole/μl) | 1 |
| BQCV Dual-labeled probe (5 pmole/μl) | 3 |
| DEPC Water | 5 |

Referring to the real-time PCR results shown in FIGS. 1 to 6, when Compound 2, Compound 5, Compound 6, Compound 7, Compound 11, Compound 12, Compound 13, Compound 50, and Compound 51 were used as dual-labeled probes, it can be seen that the Ct value was relatively low and the final amplified RFU value was high compared to the commercially available fluorescent materials Cy3™, Cy5™, and Cy5.5™.

In terms of molecular diagnostics, since the limit of detection (LoD) of a compound defined herein as a reporter for labeling a nucleic acid is lower than that of other commercially available fluorescent materials, when a compound defined herein is used as a reporter, it may be much easier to detect the target DNA or RNA compared to other commercially available fluorescent materials even when the target DNA or RNA in the sample is present at a relatively low concentration.

Referring to Table 6 below showing the linearity results measured using real-time PCR, it can be seen that when Compound 2, Compound 5, Compound 6, Compound 7, Compound 11, Compound 12, Compound 13, Compound 50, and Compound 51 were used as dual-labeled probes, they exhibited excellent linearity compared to the commercially available fluorescent materials Cy3™, Cy5™, and Cy5.5™

TABLE 6

| | DNA | Copy number | | |
|---|---|---|---|---|
| Classification | (copies/rxn) | (Log) | Ct | RFU |
| Example 1 | 3,000,000 | 6.48 | 22.31 | 20348.30 |
| | 300,000 | 5.48 | 25.62 | 20553.16 |
| | 30,000 | 4.48 | 29.12 | 19401.68 |
| | 3,000 | 3.48 | 32.28 | 18304.96 |
| Example 2 | 3,000,000 | 6.48 | 22.30 | 7379.42 |
| | 300,000 | 5.48 | 25.75 | 7763.84 |
| | 30,000 | 4.48 | 29.09 | 7406.86 |
| | 3,000 | 3.48 | 32.09 | 6698.01 |
| Example 3 | 3,000,000 | 6.48 | 21.59 | 10651.44 |
| | 300,000 | 5.48 | 24.95 | 11065.51 |
| | 30,000 | 4.48 | 28.22 | 10612.25 |
| | 3,000 | 3.48 | 31.61 | 9832.63 |
| Example 4 | 3,000,000 | 6.48 | 21.98 | 8392.59 |
| | 300,000 | 5.48 | 25.22 | 8547.10 |
| | 30,000 | 4.48 | 28.60 | 8536.10 |
| | 3,000 | 3.48 | 31.96 | 7618.57 |
| Example 5 | 3,000,000 | 6.48 | 22.13 | 22887.64 |
| | 300,000 | 5.48 | 25.65 | 20903.66 |
| | 30,000 | 4.48 | 28.89 | 20368.47 |
| | 3,000 | 3.48 | 32.30 | 19820.60 |
| Example 6 | 3,000,000 | 6.48 | 22.44 | 19200.76 |
| | 300,000 | 5.48 | 25.81 | 19462.41 |
| | 30,000 | 4.48 | 29.11 | 19246.49 |
| | 3,000 | 3.48 | 32.34 | 17240.04 |
| Example 7 | 3,000,000 | 6.48 | 22.61 | 6338.07 |
| | 300,000 | 5.48 | 26.12 | 6359.52 |
| | 30,000 | 4.48 | 29.33 | 6341.19 |
| | 3,000 | 3.48 | 32.33 | 6293.22 |
| Example 8 | 3,000,000 | 6.48 | 21.56 | 10664.79 |
| | 300,000 | 5.48 | 25.07 | 9996.94 |
| | 30,000 | 4.48 | 28.55 | 8857.95 |
| | 3,000 | 3.48 | 31.94 | 8526.70 |
| Example 9 | 3,000,000 | 6.48 | 22.06 | 8353.37 |
| | 300,000 | 5.48 | 25.61 | 8278.98 |
| | 30,000 | 4.48 | 28.76 | 8461.61 |
| | 3,000 | 3.48 | 32.10 | 7801.32 |
| Comparative | 3,000,000 | 6.48 | 22.61 | 17814.16 |
| Example 1 | 300,000 | 5.48 | 26.06 | 16944.36 |
| (C. Example 1) | 30,000 | 4.48 | 29.29 | 16986.76 |
| | 3,000 | 3.48 | 32.96 | 15081.32 |
| Comparative | 3,000,000 | 6.48 | 23.28 | 4477.92 |
| Example 2 | 300,000 | 5.48 | 26.98 | 4057.75 |
| (C. Example 2) | 30,000 | 4.48 | 30.30 | 4051.88 |
| | 3,000 | 3.48 | 33.03 | 4203.40 |
| Comparative | 3,000,000 | 6.48 | 22.36 | 6352.35 |
| Example 3 | 300,000 | 5.48 | 25.87 | 5970.11 |
| (C. Example 3) | 30,000 | 4.48 | 29.23 | 6092.57 |
| | 3,000 | 3.48 | 32.55 | 5501.55 |

When comprehensively reviewing the above results, as a reporter for labeling a nucleic acid, the compounds defined herein may be applied to conventional nucleic acid labeling and detection fields (such as PCR experiments) or may sufficiently replace existing commercially available fluorescent materials.

Since the novel compounds according to the present invention have a low limit of detection compared to existing commercially available fluorescent materials, the compounds can easily detect the target biomolecule even when the target biomolecule is present at a low concentration in the sample.

Accordingly, the novel compounds according to the present invention can be applied to the field of labeling and detecting biomolecules (such as nucleic acids or proteins) by replacing existing commercially available fluorescent materials.

Although several embodiments of the present invention have been described above, those skilled in the art can modify and change the present invention in various ways by adding, changing, or deleting components without departing from the spirit of the present invention as set forth in the claims, and such modifications are also included within the scope of the rights of the present invention.

What is claimed is:

1. A compound represented by Chemical Formula 1 below: 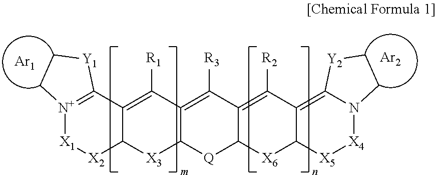
Wherein,
Ar₁ and Ar₂ are each independently a substituted or unsubstituted C₆-C₂₀ aryl or a substituted or unsubstituted C₁-C₂₀ heteroaryl containing at least one heteroatom,
Y₁ and Y₂ are each independently CRₐR_{b}, NR_{c}, O, or S,
Q is O, S, or NR_{d},
X₁ to X₆ are each independently CRₑR_{f},
Rₐ to R_{f} and R₁ to R₃ are each independently
(1) a functional group selected from hydrogen, deuterium, a substituted or unsubstituted C₁-C₄₀ alkyl, a substituted or unsubstituted C₁-C₄₀ heteroalkyl containing at least one heteroatom, a substituted or unsubstituted C₂-C₄₀ alkenyl, a substituted or unsubstituted C₂-C₄₀ alkynyl, a substituted or unsubstituted C₃-C₂₀ cycloalkyl, a substituted or unsubstituted C₃-C₂₀ cycloalkenyl, a substituted or unsubstituted C₂-C₂₀ heterocycloalkyl, hydroxy, oxido (—O⁻), a substituted or unsubstituted C₁-C₄₀ alkoxy, a substituted or unsubstituted C₃-C₄₀ cycloalkyloxy, a substituted or unsubstituted C₅-C₄₀ aryloxy, a substituted or unsubstituted C₂-C₄₀ heteroaryloxy, a substituted or unsubstituted C₅-C₅₀ aryl, a substituted or unsubstituted C₂-C₅₀ heteroaryl, a substituted or unsubstituted C₅-C₅₀ aralkyl, a substituted or unsubstituted C₁-C₄₀ alkylthio, a substituted or unsubstituted C₅-C₄₀ arylthio, a substituted or unsubstituted C₃-C₄₀ cycloalkylthio, a substituted or unsubstituted C₂-C₄₀ heteroarylthio, a substituted or unsubstituted acylamino, acyloxy, a substituted or unsubstituted phosphino, carboxylate (—CO₂⁻), trifluoromethylsulfonyl (—SO₂CF₃), a substituted or unsubstituted ammonium, nitro, sulfonic acid (—SO₃H), sulfonate, a substituted sulfonyl, a substituted sulfonic acid ester, a substituted or unsubstituted sulfonamide, a substituted thioketone, a trihalomethyl (—CF₃, —CCl₃, —CBr₃, —CI₃), a haloformyl (—COCl, —COBr, —COI), formyl (—CHO), acyl, carboxyl, a substituted ester, a substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, a substituted or unsubstituted silyl, a substituted or unsubstituted amide, carbamate, carboxylate, a substituted or unsubstituted phosphine, substituted or unsubstituted phosphoric acid, phosphate, phosphonic acid, phosphonate, nitrile, hydrazine, acetal, ketal, and polyalkylene oxide,
(2) a functional group selected from carboxyl, a carboxyl derivative, hydroxyl, a haloalkyl, a dienophile, an aldehyde, a substituted ketone, a sulfonyl halide, a thiol, amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the functional group,
(3) a reactive group capable of covalent bonding with a functional group selected from carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the reactive group, or
(4) a protecting group selected from an alcohol-derived protecting group, an amine-derived protecting group, a carbonyl-derived protecting group, a carboxylic acid-derived protecting group, a phosphate-derived protecting group, and an alkyne-derived protecting group, or any functional group substituted with the protecting group,
m and n are each independently 0 or 1, and
Rₑ and R_{f} of at least one of X₁ to X₆ are not hydrogen and deuterium.

2. The compound of claim 1, wherein the compound is represented by Chemical Formula 2 below, 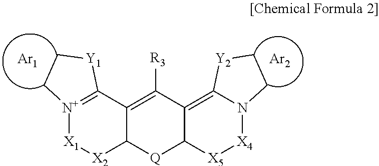
Wherein,
Rₑ and R_{f} of at least one of X₁, X₂, X₄, and X₅ are not hydrogen and deuterium.

3. The compound of claim 1, wherein the compound is represented by Chemical Formula 3 below, 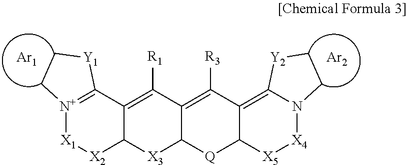
Wherein,
Rₑ and R_{f} of at least one of X₁ to X₅ are not hydrogen and deuterium.

4. The compound of claim 1, wherein the compound is represented by Chemical Formula 4 below, 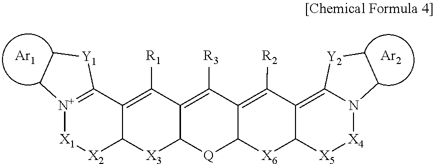
Wherein,
Rₑ and R_{f} of at least one of X₁ to X₆ are not hydrogen and deuterium.

5. The compound of claim 1, wherein at least one of a functional group bonded to any carbon of Rₐ to R_{f}, R₁ to R₃, and Ar₁ and a functional group bonded to any carbon of Ar₂ is:
(2) a functional group selected from carboxyl, a carboxyl derivative, hydroxyl, a haloalkyl, a dienophile, an aldehyde, a substituted ketone, a sulfonyl halide, a thiol, amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the functional group;
(3) a reactive group capable of covalent bonding with a functional group selected from carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the reactive group; or
(4) a protecting group selected from an alcohol-derived protecting group, an amine-derived protecting group, a carbonyl-derived protecting group, a carboxylic acid-derived protecting group, a phosphate-derived protecting group, and an alkyne-derived protecting group, or any functional group substituted with the protecting group.

6. The compound of claim 1, wherein at least one selected from Rₑ and R_{f} of at least one of X₁ to X₆ is a substituted or unsubstituted C₁-C₄₀ alkyl, a substituted or unsubstituted C₁-C₄₀ heteroalkyl containing at least one heteroatom, a substituted or unsubstituted C₃-C₂₀ cycloalkyl, a substituted or unsubstituted C₃-C₃₀ heterocycloalkyl containing at least one heteroatom, a substituted or unsubstituted C₅-C₅₀ aryl, a substituted or unsubstituted C₂-C₅₀ heteroaryl, fluoro (—F), chloro (—Cl), bromo (—Br), or iodo (—I).

7. The compound of claim 1, wherein the compound is represented by Chemical Formula 5 below, 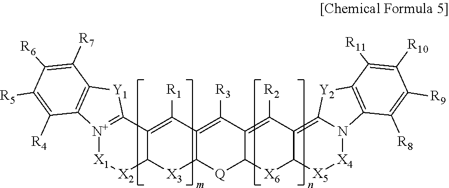
Wherein,
Y₁ and Y₂ are each independently CRₐR_{b}, NR_{c}, O, or S,
Q is O, S, or NR_{d},
X₁ to X₆ are each independently CRₑR_{f},
Rₐ to R_{f} and R₁ to R₁₁ are each independently
(1) a functional group selected from hydrogen, deuterium, a substituted or unsubstituted C₁-C₄₀ alkyl, a substituted or unsubstituted C₁-C₄₀ heteroalkyl containing at least one heteroatom, a substituted or unsubstituted C₂-C₄₀ alkenyl, a substituted or unsubstituted C₂-C₄₀ alkynyl, a substituted or unsubstituted C₃-C₂₀ cycloalkyl, a substituted or unsubstituted C₃-C₂₀ cycloalkenyl, a substituted or unsubstituted C₂-C₂₀ heterocycloalkyl, hydroxy, oxido (—O⁻), a substituted or unsubstituted C₁-C₄₀ alkoxy, a substituted or unsubstituted C₃-C₄₀ cycloalkyloxy, a substituted or unsubstituted C₅-C₄₀ aryloxy, a substituted or unsubstituted C₂-C₄₀ heteroaryloxy, a substituted or unsubstituted C₅-C₅₀ aryl, a substituted or unsubstituted C₂-C₅₀ heteroaryl, a substituted or unsubstituted C₅-C₅₀ aralkyl, a substituted or unsubstituted C₁-C₄₀ alkylthio, a substituted or unsubstituted C₅-C₄₀ arylthio, a substituted or unsubstituted C₃-C₄₀ cycloalkylthio, a substituted or unsubstituted C₂-C₄₀ heteroarylthio, a substituted or unsubstituted acylamino, acyloxy, a substituted or unsubstituted phosphino, carboxylate (—CO₂⁻), trifluoromethylsulfonyl (—SO₂CF₃), a substituted or unsubstituted ammonium, nitro, sulfonic acid (—SO₃H), sulfonate, a substituted sulfonyl, a substituted sulfonic acid ester, a substituted or unsubstituted sulfonamide, a substituted thioketone, a trihalomethyl (—CF₃, —CCl₃, —CBr₃, —CI₃), a haloformyl (—COCl, —COBr, —COI), formyl (—CHO), acyl, carboxyl, a substituted ester, a substituted or unsubstituted aminocarbonyl, nitro, nitroso (—N═O), fluoro (—F), chloro (—Cl), bromo (—Br), iodo (—I), substituted or unsubstituted germanium, substituted or unsubstituted boron, substituted or unsubstituted aluminum, a substituted or unsubstituted silyl, a substituted or unsubstituted amide, carbamate, carboxylate, a substituted or unsubstituted phosphine, substituted or unsubstituted phosphoric acid, phosphate, phosphonic acid, phosphonate, nitrile, hydrazine, acetal, ketal, and, polyalkylene oxide,
(2) a functional group selected from carboxyl, a carboxyl derivative, hydroxyl, a haloalkyl, a dienophile, an aldehyde, a substituted ketone, a sulfonyl halide, a thiol, amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the functional group,
(3) a reactive group capable of covalent bonding with a functional group selected from carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the reactive group; or
(4) a protecting group selected from an alcohol-derived protecting group, an amine-derived protecting group, a carbonyl-derived protecting group, a carboxylic acid-derived protecting group, a phosphate-derived protecting group, and an alkyne-derived protecting group, or any functional group substituted with the protecting group,
m and n are each independently 0 or 1, and
Rₑ and R_{f} of at least one of X₁ to X₆ are not hydrogen and deuterium.

8. The compound of claim 7, wherein the compound is represented by Chemical Formula 6 below, 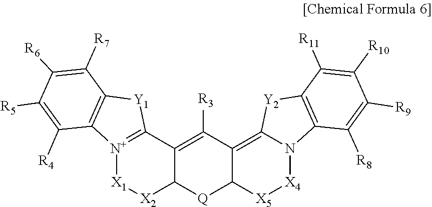
Wherein,
Rₑ and R_{f} of at least one of X₁, X₂, X₄, and X₅ are not hydrogen and deuterium.

9. The compound of claim 7, wherein the compound is represented by Chemical Formula 7 below, 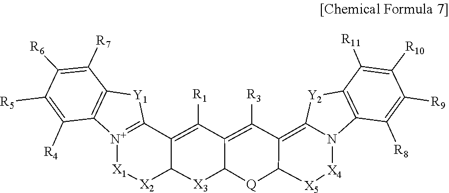
Wherein,
Rₑ and R_{f} of at least one of X₁ to X₅ are not hydrogen and deuterium.

10. The compound of claim 7, wherein the compound is represented by Chemical Formula 8 below, 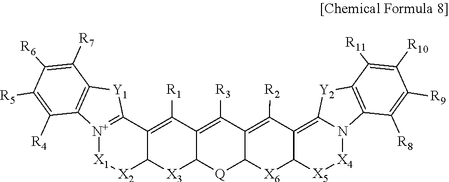
Wherein,
Rₑ and R_{f} of at least one of X₁ to X₆ are not hydrogen and deuterium.

11. The compound of claim 7, wherein at least one of Rₐ to R_{f} and R₁ to R₁₁ is:
(2) a functional group selected from carboxyl, a carboxyl derivative, hydroxyl, a haloalkyl, a dienophile, an aldehyde, a substituted ketone, a sulfonyl halide, a thiol, amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the functional group;
(3) a reactive group capable of covalent bonding with a functional group selected from carboxyl, a carboxyl derivative, hydroxyl, a haloalkyl, a dienophile, an aldehyde, a substituted ketone, a sulfonyl halide, a thiol, a substituted or unsubstituted amino, an alkene, an alkyne, a halogen, a hydrazide, azido, imido, a ketene, an isocyanate, an epoxide, a maleimide, a 1,2,4,5-tetrazine derivative, a cycloalkyne derivative, a cycloalkene, a triphosphate, and a phosphoramidite, or any functional group substituted with the reactive group; or
(4) a protecting group selected from an alcohol-derived protecting group, an amine-derived protecting group, a carbonyl-derived protecting group, a carboxylic acid-derived protecting group, a phosphate-derived protecting group, and an alkyne-derived protecting group, or any functional group substituted with the protecting group.

12. The compound of claim 7, wherein two adjacent functional groups among R₄ to R₁₁ are bonded to each other to form a ring.

13. The compound of claim 1, wherein Q is O, and Y₁ and Y₂ are each independently selected from CRₐR_{b}, O, or S.

14. The compound of claim 1, wherein the compound is a reporter for labeling a nucleic acid.

15. An oligonucleotide comprising:
the compound of claim 1 as a reporter for labeling a nucleic acid; and
a quencher.

16. The oligonucleotide of claim 15, wherein a minor groove binder (MGB) is interposed between the compound and the quencher.

17. A composition for detecting a nucleic acid, comprising the oligonucleotide of claim 15.

18. A support for detecting a nucleic acid, comprising:
a probe singly labeled with the compound of claim 1 or a probe dually labeled with the compound of claim 1 and a quencher;
a support; and
a linker connecting the probe and the support.

19. The support of claim 18, wherein the support is glass, cellulose, nylon, acrylamide gel, dextran, polystyrene, or a resin.

20. The support of claim 18, wherein the linker is selected from a substituted or unsubstituted C₁-C₃₀ alkyl, a substituted or unsubstituted C₃-C₃₀ cycloalkyl, a substituted or unsubstituted C₂-C₃₀ heteroalkyl containing at least one heteroatom, a substituted or unsubstituted C₂-C₃₀ heterocycloalkyl containing at least one heteroatom, a substituted or unsubstituted C₂-C₃₀ alkenyl, a substituted or unsubstituted C₆-C₃₀ aryl, a substituted or unsubstituted C₃-C₃₀ heteroaryl, amide (—CONH—), ester (—COO—), ketone (—CO—), a nucleoside, and any combination thereof.

21. A method of detecting a nucleic acid, comprising:
(a) preparing a reaction mixture including a target nucleic acid, a reagent required for amplifying the target nucleic acid, and the oligonucleotide of claim 15;
(b) amplifying the target nucleic acid in the reaction mixture through polymerase chain reaction; and
(c) measuring a fluorescence intensity of the reaction mixture.

22. The method of claim 21, wherein (b) includes:
(b-1) elongating the oligonucleotide hybridized to the target nucleic acid by a polymerase;
(b-2) separating the reporter and the quencher of the oligonucleotide from the target nucleic acid by the exonuclease activity of the polymerase; and
(b-3) allowing the reporter separated from the quencher to emit fluorescence.

23. The method of claim 21, further comprising (d) measuring an amplification amount of the target nucleic acid from the fluorescence intensity measured in (c).

24. The compound of claim 1, wherein the compound is a dye for labeling proteins.
